(19) United States
(12) Patent Application Publication
(10) Pub. No.: US 2022/0025119 A1
(43) Pub. Date: Jan. 27, 2022

(54) WASTE MITIGATION METHODS AND MATERIALS

(71) Applicant: Shenshen Li, Plano, TX (US)

(72) Inventor: Shenshen Li, Plano, TX (US)

(21) Appl. No.: 17/384,807

(22) Filed: Jul. 26, 2021

Related U.S. Application Data
(60) Provisional application No. 63/056,667, filed on Jul. 26, 2020.
(60) Provisional application No. 63/168,884, filed on Mar. 31, 2021.

(51) Int. Cl. C08G 69/48; C08G 73/10

(52) U.S. Cl. CPC C08G 69/48; C08G 73/10

(57) ABSTRACT

A compound, comprising a segment having the formula (AA) or (BB):

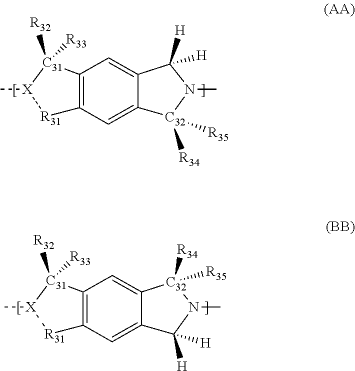

wherein C₃₁ and C₃₂ are carbons (C),
wherein R₃₂ and R₃₃ are both hydrogens or collectively forms a carbonyl with C₃₁,
wherein R₃₄ and R₃₅ are both hydrogens or collectively forms a carbonyl with C₃₂,
wherein X is oxygen (O) or nitrogen attached to an organic residue,
wherein R₃₁ is a hydrogen, a hydroxymethyl, a halogen-substituted methyl, or an unsubstituted methylene —CH₂— group covalently and directly bonded to X,
wherein dashed lines (---) represent optional covalent bonds to satisfy valence.

### CROSS REFERENCES TO RELATED APPLICATIONS

This present application claims priority to and the benefit of U.S. Provisional Application No. 63/056,667 filed Jul. 26, 2020 and 63/168,884 filed on Mar. 31, 2021, which are hereby incorporated by reference in their entirety as if fully set forth below and for all applicable purposes.

### TECHNICAL FIELD

Embodiments of this present invention relate to waste mitigation methods and materials. More specifically, embodiments of this present invention relate to radical scavengers capable of stabilizing polymers, OS capable of extending shelf-lives of packaged contents, recycling methods capable of extending useful lives of the plastics materials, and recycling methods that produce value-added products.

### DETAILED DESCRIPTION

Traditionally, environmentalists advocate a curbside recycling program where these solid wastes are first collected, and then sent to Materials Recovery Facilities (“MRFs”) for sorting and reuse.

Clean MRFs recover single-streams of recyclable materials, such as polyethylene terephthalate (“PET”), high density polyethylene (“HDPE”), mixed paper, etc., from source-separated streams, while Mixed-Waste Processing Facilities (“MWPFs”, or “dirty MRFs”) go through a more intensive manual and mechanical sorting to reclaim valuables with the remainder being sent to the landfill. Both types of MRFs operate at significant costs, either due to the limited availability of source-sorted waste materials, or the labor-intensive separations and treatment processes. Sometimes, recycling of two otherwise recyclable materials is made impossible merely because they are mixed together. For example, 50 billion paper coffee cups are disposed into landfills each year due to the fact it is almost impossible to separate the small amount of plastics embedded in the paper cups. What has further worsened the situation is that materials otherwise recyclable are often rendered incompatible due to additives or pigments they are used with. For example, HDPEs in mixed colors are often difficult to recycle without separation. Similarly, while wood products are generally considered eco-friendly and recyclable, plywood with paints on them often become problematic in the recycling stream. Similar problems exist for colored ceilings, colored plastics bottles, pigmented pipes and cables, etc. Due to the large amount of the colorants and pigments inside, they are typically rejected at the recycling facilities. It is therefore desired to have a more efficient recycling methodology for these municipal solid wastes, as well as other wastes of similar characters, especially those strongly colored or with large amounts of additives. Besides the challenge faced by the recycling industry against solid wastes, water and soil pollutions are no less urgent. While part of their problem stems from landfills, direct pollutions are more staggering. It is urgent to find an effective method to decontaminate these polluted water and soils on a large scale. Furthermore, food wastes and agricultural waste is also staggering. In 2010, the food waste is estimated to be at between 30% to 40% in the US. Packaging materials (e.g. plastic packaging) have become indispensable in this area. Despite their remarkable performances, they still require assistance from additives in e.g. shelf-life improvement of food, beverage, drug, and similar products. Such additives have not been satisfactory in all aspects due to their deficiencies in safety, aesthetics, and/or effectiveness. Moreover, the use of these additives has been shown to degrade the recycling stream of the plastics materials thereby further aggravating the plastics crisis.

Therefore, an improved system addressing the interrelated problems of solid wastes, plastics recycling, plastics additives, water/soil depollution, are highly desirable. This present disclosure provides an integrated solution to these problems. For example, the present disclosure provides oxygen (O₂) scavenger materials to increase shelf-life of commodities; radical scavengers that reduce plastics degradations thereby extending their useful lives and improve their performances during mechanical recycling; and chemical recycling methods that produce plastics raw starting materials as well as value-added materials useful in combating food wastes, environmental disasters (such as oil spills), water and soil pollutions thereby improving upon otherwise non-favorable economics and further solving other societal problems. Additionally, the present disclosure provides novel solutions to other challenges encountered in related industries. We previously filed related patent applications PCT/US20/15209 on Jan. 27, 2020, provisional application 62/797,269 on Jan. 26, 2019, and provisional application 62/797,277 on Jan. 27, 2019, collectively referred to as “related applications”, all of which are herein incorporated by reference in their entireties.

Oxygen scavengers (OS) are a class of organic molecules that are capable of reacting with molecular oxygen (O₂) and thereby removing the O₂ from the system at ambient or near ambient temperatures. They are effective solutions for extending shelf-lives of contents they contain. Moreover, O₂ absorbers, O₂ removers, O₂ scrubbers, O₂ barrier materials (more specifically, the active-type O₂ barrier), oxidizable material, used in various diverse applications may share similar chemical mechanisms with the OS. These materials are collectively referred to as “O₂ scavengers” or “OS” herein. OS also includes all “oxidizable additives” described in the related applications. OS may be present in the system as pure scavenger compounds, scavenger compositions that include one or more catalysts, scavenger compositions that include one or more additional additives. They may constitute majority of the composition (such as 80%-100%), or only a small fraction of the compositions (such as 0.1% to 10%), or anything in between. These OS may be used to remove headspace oxygen from packaging articles (or containers, trays, cartons, or any similar names) for food, beverage, drug, chemicals, vaccines, proteins, other pharmaceutical products, agricultural products, or other oxygen-sensitive materials, thereby extending their shelf lives. The packaging article may be rigid plastics or flexible plastics. Moreover, OS may or may not be formally processed into a packaging article. For example, plastics films may be processed to include such OS, and applied to wrap (loosely or tightly) certain bulky agricultural products. The wrapped films may or may not maintain openings towards the outside. This is still contemplated by the present disclosure. The ambient or near ambient temperature may include lightly frozen temperatures (e.g. about −20° C. to about 0° C.), refrigerated temperatures (about 0° C. to about 10° C.), room temperature, and slightly higher temperatures (such as up to about 85° C.). The higher temperature application may be those such as “hot tea” or “hot coffee” applications. The lower temperature application may be those food and beverage that require cold storage or cold transportation, and pharmaceutical products that require low temperature storage. Furthermore, many of the present application that require low-temperature storage or transportation, the temperature requirement may be relaxed if OS is implemented to protect them from O₂ degradations.

For example, recently developed COVID-19 vaccine candidates require chemical stability against O₂. Many other vaccines, proteins, antibody, small molecule drugs, biologics, biosimilars, other biological products similarly require chemical stability against O₂, sometimes at low temperatures. Accordingly, OS described herein (and those described in the '209 application) may be useful or even crucial in their storage and/or transportations. For example, OS may be used in the packaging article that surrounds these contents (either as an additive to the packaging article wall, lid, or in the form of a sachet, a packet, or an insert) and thereby stopping O₂ ingression into the article such that the contents are not exposed to O₂. Alternatively, OS may be incorporated as part of the formulation to these contents and coexist in the active formulation. Similarly, consumer food and beverage products often require OS to maintain their freshness over an extended period of time. This not only has economic consequences for the manufacturers, or the industries, such as from reducing spoilage and waste, but also societal impacts such as sustainability and/or resources conservations. OS may be used in similar or dissimilar ways to achieve the target. As described above, certain types of OS described herein may be particularly suitable for lightly frozen or refrigerated conditions (such as certain particularly easy-to-spoil food applications or sensitive drug applications) in part due to their particularly high activity. Certain types of OS may be particularly suitable for a slightly elevated temperatures (such as “hot tea” “hot chocolate” and “hot coffee” temperatures) in part due to their large capacity. Moreover, OS may be used in inorganic solar cells, vacuum insulating panels, flexible LCDs, flexible OLED, and organic solar cells to maintain oxygen-free working environments, with the latter ones being more sensitive to oxygen and require more OS and/or more active OS. OS may even be used in sealants for aerospace applications.

Definitions from the '209 application are adopted in this present disclosure, except those in conflict. If there is a conflict in the interpretation of a term in the '209 application (e.g. definition 1) and the rest of this disclosure (e.g. definition 2), the definition provided by the '209 application (e.g. definition 1) governs the portion incorporated from the '209 application, while the other definition (e.g. definition 2) governs the rest of the present disclosure. Further, although not specifically described below, precursors to “oxidizable additives” (which are also “OS”) can be used in place of the OS, provided that they are capable of being converted to the OS under normal processing or application conditions to satisfy the intended requirement of OS reactivity. These precursors may include any suitable catalysts (e.g. “exchange catalysts” for catalyzing “exchange reactions” which include transesterification, transesteramidification, or transamidification catalyst, etc.). Still further, this present application provides additional embodiments of structures capable of serving as OS with the presence (and sometimes without the presence) of a radical catalyst, and capable of serving as radical scavengers (RS) in absence of the radical catalyst. The term repeating unit refers to a ditopic segment (e.g. a diradical) of an oligomer, a polymer, or a copolymer, that appears repetitively, and may or may not correspond to a monomer or comonomer used in synthesis. The term “radical” may refer to the species with unpaired electrons, or may refer to organic residues that are connected to other residues, as the context may require. Moreover, the term may be used to refer to one of the multiple comonomer units. In some embodiments, the term amine (including diamine, triamine, polyamine, polyetheramine, or the like) does not include amides, unless specifically stated that it directly bonds to a carbonyl. Likewise, the term hydroxy (including diol, triol, polyol, polyetheramine) does not include ester, unless specifically stated to directly bonded to a carbonyl. In some embodiments, the term ditopic includes tritopic, which includes tetratopic and polytopic. Likewise, the term diamine and diol includes triamine and triol, respectively, and the term triamine and triol includes tetraamine and tetraol, respectively, and so on, and they all include polyamine or polyol, respectively.

Embodiments below describe methods and materials to address various challenges using polyethylene terephthalate (PET) as an example. However, this is not intended to be limiting. Other similar polyesters (e.g. polybutylene terephthalate (PBT), polyethylene furanoate (PEF)), polyamides, etc., share many of the same characteristics as PET and may implement the same or similar methods or materials described here. Moreover, other polymers (e.g. polyethylene, polypropylene, polystyrene, polycarbonate, polyurethane, polyvinylchloride, polylactic acid, polyglycolic acids, polyacrylate, acrylonitrile butadiene styrene, rubbers, etc. may also benefit from certain aspects of the present disclosure without departing from the spirit thereof. Furthermore, the term “plastics” as used herein may broadly include thermoplastics, thermosets, thermoelastics (e.g. thermoplastic elastomers), and the similar.

In an embodiment, the OS may be coextruded with the PET as a standalone additive, which provides the benefit of portability (e.g. it may be used with different grades of PET or non-PET resins). However, the migration and toxicity profile of the OS may render this implementation unsuitable for certain applications. In an embodiment, the OS may be used as a comonomer or copolymer with PET, which in some instances reduces the migration of the OS (or byproducts from OS) and alleviates any toxicity concerns. PET with such OS may be less prone to oxidative degradation (or other mechanisms with an oxidative component, e.g. hydrolytic oxidative degradation) during the processing and usage. For example, the OS may include one or more OS that includes an organic moiety including a first carbon atom (referred to here as C₁) directly attached to a hydrogen atom (H) forming a C₁—H bond, and the first carbon atom further being directly attached to and forming covalent bonds with (1) each of a first group, a second group, and a third group, or (2) each of a strong mesomeric electron-donating group and a strong mesomeric electron withdrawing group. Because the C₁ atom is generally active towards reacting with O₂, the C₁ atom may be referred to as the active site. The first group includes a conjugated unit selected from a double bond, a triple bond, an aromatic ring. The first group further includes a first anchor atom (AA). The first anchor atom has an sp² hybridization, an sp hybridization, or a lone pair of valence electrons. The first group is directly attached to the first carbon atom (C₁) at the first anchor atom, forming a covalent C₁-AA bond. The second group includes a heteroatom and is selected from a triple bond, a C═N unit, a N═O unit, a first C═O unit directly attached to the first carbon atom and a second carbon atom forming C₁—C(═O)—C, a second C═O unit directly attached to the first carbon atom and an oxygen forming C₁—C(═O)—O, a third C═O unit directly attached to the first carbon atom and a first nitrogen atom (said first nitrogen atom being directly attached to a third carbon atom) forming C₁—C(═O)—N—, a first fragment directly attached to the first carbon atom at an oxygen forming C₁—O, a second fragment directly attached to the first carbon atom at a nitrogen forming C₁—N, and a third fragment having at least three heteroatoms within a spatial distance of 4 Å from the first carbon atom (the three heteroatoms includes a nitrogen)—provided that the second group is the third fragment if and only if the first group is an ester directly attached to the first carbon atom with an ester oxygen or an amide directly attached to the first carbon atom with an amide nitrogen. The third group is selected from a hydrogen, an alkyl group, an aromatic group, a double bond, a triple bond, and a heteroatom. to some embodiments, when the first group is a benzene or a vinyl, the third group does not form a ring containing the first carbon atom and the first anchor atom. In some embodiments, when the first carbon atom is directly attached to a carbonyl group and directly attached to an oxygen atom forming C(═O)—C₁—O, (1) the oxygen atom is directly attached to one of hydrogen and a double bond, (2) the first carbon atom is further directly attached to one of a hydrogen, a double bond. and an oxygen, or (3) the carbonyl group is directly attached to a double bond. In some embodiments, when the first carbon atom is directly attached to a vinyl and to a chalcogen selected from an oxygen, a sulfur, and a selenium, (1) the chalcogen is directly attached to one of a hydrogen, a heteroatom, a triple bond, and a linear alkyl with more than four carbon atoms, (2) the vinyl is directly attached to one of a heteroatom and a double bond having a heteroatom, or (3) the first carbon is directly attached to one of a heteroatom separated from the chalcogen, and a double bond having a heteroatom separated from the chalcogen. In some embodiments, when the first carbon atom is directly attached to a benzene and directly attached to an oxygen, (1) the oxygen is directly attached to one of a hydrogen, a vinyl, and a carbonyl directly attached to a vinyl, or (2) the first carbon atom is further attached to a carbonyl group. in some embodiments, when the first carbon atom is directly attached to a benzene or a vinyl and directly attached to a nitrogen atom, (1) the nitrogen atom is directly attached to a carbonyl of an acetylbenzoate (—C(═O)-p-C₆H₄—C(═O)—O—) moiety or directly attached to one of a linear alkyl having more than 4 carbons, an aromatic group, and an allyl, or (2) the first carbon atom is further directly attached to a carbonyl group. In some embodiments, the strong mesomeric electron-donating group recited above is selected from a phenoxide (—O⁻) group, an amine (—NR₂, —NHR, —NH₂) group, an ether (—OR) group, and a hydroxy (—OH) group. In some embodiments, the strong mesomeric electron-withdrawing group recited above is selected from a cyano (CN) group, a triflyl (—SO₂CF₃) group, a sulfonate (—SO₃H) group, and a nitro-(—NO₂) group. Any carbon atoms whose bonding structure conform to the above criteria may be referred to as a C₁ active site (or simply C₁ site). In some embodiments, a molecule may include multiple C₁ carbon atoms. Each of the multiple carbon atoms may be evaluated separately and independently from each other against the above criteria. If more than such carbon atoms conform to the C₁ criteria, the molecule may include more than one C₁ active sites for the oxygen scavenging (and radical scavenging) reactions. Generally, all things equal, a molecule having more C₁ sites may have better reactivity towards oxygen than molecules having fewer C₁ sites. In an embodiment, a precursor to OS may similarly implemented which is converted into the OS during processing or application of the composition.

In an embodiment, the OS may include N,N′-[1,3-phenylenebis(methylene)]bis(isoindolin-1-one) (33), poly[(1,3-phenylene)bis(methylene)adipamide] (34), poly(1,4-butadiene) (35), condensation product of benzoic acid (or other carboxylic acid) and poly(1,4-butyleneglycol) (sometimes referred to as poly(tetrahydrofuran)) (36), tribenzylamine (37), or the like. In an embodiment, the C₁ of the OS is part of a ring structure. The C₁ site is sometimes subject to oxidative bond cleavages. By embedding the C₁ in a ring structure, the risk of complete fragmentation and release into the contents of the packaging is reduced. In an embodiment, the ring structure includes a heteroatom. In an embodiment, the C₁ directly bonds to the heteroatom and an sp² carbon. For example, the ring structure includes a 2,5-dihydrofuran (1) motif, a 1,3-dihydro-2-benzofuran (2) motif, an isoindoline motif (3), a 2,5-dihydropyrrole (4) motif. For another example, the ring structure includes a 1,3-dihydro-2H-inden-2-one (5) motif, a cyclopent-3-en-1-one (6) motif, a cyclopenta-3-en-1-imine (7) motif In an embodiment, the ring structure includes two C₁ sites, for example, when the ring structure of motifs 1-7 includes two sp³ carbon atoms, both of which are attached to at least one hydrogen. Both these two sp³ carbon may function as the C₁ activation center. In an embodiment, the ring structure includes only one C₁ site, for example, when at least one of those two sp³ carbons are disubstituted (e.g. phthalide (8), isoindolin-1-one (9), etc.) For yet another example, the ring structure includes a lactone (e.g. 2-coumaranone (31)) or lactam (e.g. oxindole (32)). The above examples describe 5-member rings as mere examples. Rings of other numbers (e.g. 6-member or 7-member rings) may be implemented as well. For example, the OS may include homophthalic acid anhydride or 3-isochromanone. Rings of 4-members may be more active which may in some circumstances lead to improved activity, although in some circumstances may be too unstable to be productive. In an embodiment, the ring structure is further part of a polymeric backbone.

In an embodiment, the OS includes a trivalent heteroatom (such as N or P). In an embodiment, the trivalent atom is N. In an embodiment, the N is attached to at least one atom that does not conform to the criteria of C₁ site described above. In an embodiment, this mitigates degradations of the OS. For example, the OS may include N,N,N′,N′-Tetrabenzylethylenediamine (38). The carbon atom of the ethylene group that directly attaches to the nitrogen atom does not conform to the criteria of C₁ site and is not an active site for OS reactions. This carbon atom is significantly less prone to oxidative cleavage than C₁ atoms. Accordingly, OS include such non-C₁ atoms connected to the N may generate less low molecular degradants, or the degradants generated may have higher molecular weight than when N is connected to three C₁ sites. Applying the same concept, for example, the OS may include an isoindoline ring structure, where the N atom is bonded directly to two C₁ atoms, each being part of a benzyl group (referred to as the C₁ₐ and C_{1b}). The same N atom is also directly bonded to a third group at an atom A, which is not a C₁ site. For instance, the third group may be a substituted or unsubstituted alkyl group (e.g. ethyl, propyl, 3-phenylpropyl, 2-hydroxyethyl, etc.). Under operation conditions, C₁ₐ—N and C_{1b}—N bonds are both subject to oxidative cleavage. However, because C₁ₐ and C_{1b} are part of a ring structure, where probability of simultaneous cleavages is low, fragmentation may be less likely. Meanwhile, because A is not a C₁ atom, the N-A bond is not prone to oxidative cleavage. Therefore, the risk for forming molecular fragments is substantially reduced as compared to situations where the N is directly bonded to three C₁ atoms. For example, the OS may be N,N′-ethylene-bisisoindoline (39). For example, the OS may be N,N′-ethylene-bis(isoindolin-1-one) (72). In some embodiments, the third group may include a carbon chain of at least four carbons, for example, by replacing the ethylene linkage of 38, 39, or 72 with four-carbon linkage (e.g. that of butylenediamine) or longer. These longer linkages may improve OS reactivity due to chain that positions more active sites adjacent to each other for synergistic effects. In some embodiments, the third group may include carbonyls directly connected to trivalent element, for example, forming diamide linkages. For example, OS may be N,N′-hexanedioyl-bisisoindoline (75). Any other suitable ditopic linkages are contemplated within the scope of the disclosure.

In an embodiment, the OS may be designed to, upon oxidation, convert to a structure similar to the backbone of the polymer (here, PET having benzoate motifs and ethylene motifs). For example, the OS may include the motif 2 or 8. These motifs convert to a benzoate up oxidation, which do not substantially change properties of the PET base polymer. Accordingly, the inclusion of these motifs does not disrupt the PET recycling stream. This is a very important advantage over other OS. For example, the PET including these OS may claim a No. 1 recycling code rather than a No. 7 recycling code. For another example, the OS may be designed to include the motifs 3 or 9. These motifs convert to a benzamide which at low concentration does not substantially change properties of the PET base polymer. In an embodiment, the OS includes an isoindoline motif where the N is further connected to a 2-hydroxyethyl group. As described above, the ethyl group are not prone to oxidative cleavage, and the additional hydroxy may attach to the PET backbone either in PET synthesis, coextrusion, or in preparing OS-pending PET oligomers (e.g. by causing an end group of the PET structure to condense with a functional group on the OS or causing a transesterification or transesteramidification reaction between a functional group of the OS and a functional group of the PET structure during PET manufacturing, processing, or recycling). The OS-pending PET oligomer or polymer may be similar to masterbatches to improve stability and processability. In an embodiment, the OS includes N-(2-hydroxyethyl)isoindoline (10). To synthesize 10, commercially available N-(2-hydroxyethyl) phthalimide (15.38 mmol) is dissolved in dry THF (100 mL) and cooled to 0° C., and 1 M LiAlH₄ in THF (46 mL, 46 mmol) is added dropwise. After stirring at 0° C. for 30 min, the reaction mixture is diluted with THF (200 mL) and quenched with a saturated solution of Na2SO4 (5 mL). The organic layer is passed through a pad of silica gel, and the solvent is removed under reduced pressure to give the crude product. The crude product is chromatographed on a silica gel column under nitrogen using a gradient of hexane/ethyl acetate. Alternatively, diborane from boron trifluoride etherate (5 cm³)—slowly dropped into a suspension of 1.5 g sodium borohydride in 5 cm³ diglyme—is bubbled into a suspension of 2.3 mmol of the phthalimide in 60 cm³ dry THF. The mixture is stirred for 72 h at room temperature and the excess diborane is decomposed by addition of 0.1N HCl until pH 2 was reached, and stirred for an additional 2 h period. 1N NaOH is added until pH 10 is reached and the solution is then extracted with 330 cm³ CH₂Cl₂. The combined extracts are washed with 330 cm³ H2O, dried (Na₂SO₄), and evaporated in vacuum. The solid residue is dissolved in a small volume of CH₂Cl₂ and filtered through a silica-gel column, packed and pre-washed with the same solvent. After evaporation of the solvent, the solid residue is recrystallized from MeOH—H₂O to provide 10. In some embodiments, structures incorporating 2 or 3 may be advantageous due to the increased activity and/or capacity as compared to those incorporating 8 or 9. In some other embodiments, those incorporating 8 or 9 may be advantageous due to their lower raw material costs.

In an embodiment, the OS may be or include 1,3,5,7-tetrahydrobenzo[1,2-c:4,5-c′]difuran (40) (which may be synthesized by reducing pyromellitic anhydride using common chemical reductant such as LiAlH₄, NaBH₄, H₂, etc., similar to the synthesis of 10 above, or by using electrochemical methods). Accordingly, these OS convert to pyromellitic dianhydride, pyromellitic acid, or pyromellitic esters upon oxidation, or partially oxidized intermediates thereof. Moreover, the carboxylic acid groups produced may interact with the PET endgroups and undergo a transesterification reaction such that the pyromellitic component is incorporated into the PET backbone in place of the terephthalic acid, especially where an exchange catalyst (which is different from a radical catalyst) is present. As a result, the characteristics of the PET is preserved. Furthermore, the molecular weight and inherent viscosity may increase due to the presence of the pyromellitic core offering more than two connection points. In other words, the product of the scavenging reaction substantially resembles PET structure and may further improve the PET backbone structure. In an embodiment, the OS may include 5-carboxyphthalane (41) or 5-hydroxyethylphthalane (42) (which can be formed by chemically reducing trimellitic anhydride similar to the 10 described above). In an embodiment, the extra carboxyl or hydroxy group may be used to tether the OS onto PET backbone either in the PET manufacturing process to form barrier grade resin, coextruded with PET, or form OS-pending PET oligomers or polymers. In an embodiment, the OS includes a 1,3,5,7-tetrahydropyrrolo[3,4-f]isoindole (43⁰) diradical motif. Similarly, upon scavenging reactions, the OS converts to pyromellitic diimide, pyromellitic acid diamide diester, partially hydrolyzed intermediates, or partially oxidized intermediates thereof. Moreover, the carboxamide groups produced may interact with the PET endgroups and undergo a transesterification, especially when a transesterification catalyst is present. In an embodiment, the OS includes 2,6-bis-(2-hydroxy-ethyl)-1,3,5,7-tetrahydropyrrolo[3,4-f]isoindole (11). It may be synthesized as follows. First, commercially available pyromellitic anhydride (0.25 mole) and freshly distilled monoethanolamine (0.5 mole) are placed in a 1 L round-bottomed flask and heated on a steam bath for 30 minutes. The reaction mixture is cooled to room temperature, and recrystallized from 250 ml of boiling water to afford intermediate 2,6-bis-(2-hydroxy-ethyl)-1,3,5,7-tetrahydropyrrolo[3,4-f]isoindole-1,3,5,7-tetraone (73). The intermediate is then reduced to form the 11 following procedures similar to that described above with respect to 10 or under electrochemical conditions. In an embodiment, the OS may be 2,6-bisethyl-1,3,5,7-tetrahydropyrrolo[3,4-f]isoindole (44), which is similarly synthesized with the exception that ethylamine replaces the monoethanolamine.

The 11 may be incorporated at the end of the PET manufacturing, or may react with PET oligomers to provide OS-pending (or OS-embedded) PET oligomers (e.g. having —[CH₂—CH₂O—C(═O)-Ph-C(═O)—O]ₙ—CH₂—CH₂—CH₂—N—) fragment where the N is an isoindoline nitrogen of 11⁰, 11⁰ being the diradical of 11 missing the two end hydroxy hydrogens, and n is at least 2) and used similarly as masterbatches. Alternatively, due to the diol nature, it may also be used as a comonomer, along with ethylene glycol, to manufacture PET-11 copolymer (e.g. having —[CH₂—CH₂O—C(═O)-Ph-C(═O)—O]ₐ—[CH₂—CH₂-11⁰-Ph-C(═O)—O]_{b}— structures, where a and b are integers). PET-11 copolymer may be prepared as a block copolymer or a random copolymer. In some embodiments, 11 may be incorporated as a comonomer at a concentration by weight of less than about 20%, such as about 0.1% to about 10%. In an embodiment, 11 may be incorporated at a concentration by weight of about 1% to about 5%. At this concentration, the PET-11 copolymer may claim the same No. 1 recycling code rather than the common No. 7 recycling code available to barrier resins. If the concentration is too small, the scavenging capability may be insufficient; if too large, the product may have properties significantly modified as compared to PET, thereby introducing unnecessary incompatibility in some instances. In some embodiments, 11 may also be used as an independent additive and be co-extruded with PET in processing (e.g. similar to colorants in injection molding process), where some condensation between the PET and 11 may also occur, Moreover, as compared to many other OS, 11 produces less degradation product due to the amine-ethylene linkage, as described above, while also providing higher OS activities (due to the increased amount of active sites).

PET-11 copolymers may be prepared using well-known copolymerization methods (see e.g. EP0994909A1). In an embodiment, PET-11 may be used as a barrier grade resin for food, beverage, or drug packaging applications. It may claim a No. 1 recycling code. In one method of preparation, terephthalic acid (TA, 1 eq.), ethylene glycol (EG, 1.02 eq.), and 11 (0.027 eq.) are introduced into a batch reactor equipped with a paddle agitator along with germanium dioxide (0.05 wt % of TA). The esterification is conducted at 240° C. for 3-4 h under a nitrogen atmosphere, and the water produced from the reaction is removed by in situ distillation. When the amount of water collected reaches 95% of the theoretical value, the esterification reaction is stopped and the mixture is maintained at 260° C. for 45 min under a slight vacuum (50 Pa); then the vacuum is increased, and the reactants are stirred with a paddle agitator (50 Hz) for 50 min. After the polycondensation reaction is completed, the as-synthesized PET-11 resin is unloaded at 240° C. and quenched in cold water with a pressure of nitrogen. In an embodiment, PET-11 may include 11 at a higher concentration and be used as a barrier masterbatch. It may claim a No. 1 recycling code. It may be synthesized similarly except that 11 (0.19 eq.) and EG (0.86 eq.) are mixed along with TA (1 eq.) at the beginning of the reaction. In an embodiment, instead of comonomer, 11 is used as the sole diol monomer without EG. The process otherwise follows conventional methods of polyester preparation. The resultant polymer having a structure of —[CH₂—CH₂-11⁰-Ph-C(═O)—O]ₙ— may be used as a polymeric OS or as a barrier polymer itself. All embodiments described above with respect to 11's interaction with PET may be similarly applied to other OS described above, or below. In some embodiments, the 11 described above in each scenario may all be replaced by 2,6-bis-(2-hydroxy-ethyl)-1,5-dihydropyrrolo[3,4-f]isoindole-3,7-dione (77). As compared to 11, 77 may be synthesized at a lower cost, but also has less activity and capacity. In some embodiments, 77 may be synthesized as follows: Terephthalic acid is reacted with paraformaldehyde in fuming sulfuric acid (with about 20% to 25% SO₃) at a temperature of about 125° C. to about 150° C. for about 4 to about 20 hours. The crude product is separated and purified to prepare 1,5-dihydrofuro[3,4-4][2]benzofuran-3,7-dione (78). 78 is reacted with excess amount of ethanolamine to prepared 77. In some embodiments, the OS may be 2,6-dibenzoyl-1,3,5,7-tetrahydropyrrolo[3,4-f]isoindole (79) or 2,6-dibenzyl-1,3,5,7-tetrahydropyrrolo[3,4-f]isoindole (80). 79 may be synthesized by first reducing pyromellitic diimide 1,2,3,5,6,7-hexahydropyrrolo[3,4-f]isoindole (43), and then reacting with benzoic acid. 80 may be synthesized by further reducing 79.

In some embodiments, 43 reacts with phthalic anhydride (2 eq.) in alkaline condition (e.g. in presence of NaOH/H₂O (8 eq.) and subsequently acidified to form 2,6-di(o-carboxybenzoyl)-1,3,5,7-tetrahydropyrrolo[3,4-f]isoindole (81). In some embodiments, phthalic anhydride (2 eq.) may be replaced with trimellitic anhydride (2 eq.) to form 2,6-di(o,m-dicarboxybenzoyl)-1,3,5,7-tetrahydropyrrolo[3,4-f]isoindole or o-, p-isomers thereof (e.g, the four carboxy group arranged in o,m/o,m configuration, o,p/o,p configuration, or o,m/o,p configuration), all such isomers collectively referred to as 82. In some embodiments, only 1 eq. of trimellitic anhydride is used to form 2-(o,m-dicarboxybenzoyl)-1,3,5,6,7-pentahydropyrrolo[3,4-f]isoindole or o-, p-isomers thereof (collectively referred to as 83, In some embodiments, 82 and 83 may functional as a comonomer due to the four and two carboxylic groups capable of reacting with ethylene glycol in PET synthesis. Moreover, 82 may provide additional advantage of increasing the molecular weight of the polymer due to its four condensation sites. In some embodiments, the 43⁰ moiety of these compounds may be replaced with 3,5,7-trihydropyrrolo[3,4-f]isoindole-1-one (84⁰), 3,7-dihydropyrrolo[3,4-f]isoindole-1,5-dione (85⁰), 5,7-tetrahydropyrrolo[3,4-f]isoindole-1,3-dione (86⁰), 3,5-tetrahydropyrrolo[3,4-f]isoindole-1,7-dione(87⁰), or 7-tetrahydropyrrolo[3,4-f]isoindole-1,3,5-trione (88⁰) diradical motif. In some embodiments, the 43⁰ moiety of these compounds may be replaced with 4-(aminomethyl)isoindolin (89⁰), 4-(aminomethyl)isoindolin-1-one (90⁰), 5-(aminomethyl)isoindolin (91⁰) diradical motif, or similar. In some embodiments, the amide group of these diradical motif may be subject to reduction (e.g. with LiAlH₄) either before or after their incorporation into the OS structure (e.g. by condensation). In some embodiments, 43 may be replaced with any ditopic groups with two amine terminals, such as with xylene diamine, Alternatively, 43 may be replaced with 2,6-bis-(2-aminoethyl)-1,3,5,7-tetrahydropyrrolo[3,4-f]isoindole (18) or analogues thereof having 1˜3 of the 1,3,5,7 positions being substituted with ═O. In some embodiments, 43 may be replaced with monoamines to form molecules with OS moiety as well as one carboxy end group. These molecules may particularly be suitable to serve as end caps, In some embodiments, such monoamines may be secondary amines, such as dibenzylamine or diallylamine. For example, diallylamine may react with phthalic anhydride in presence of excess amount of NaOH in aqueous solution to form 2-(diallylcarbamoyl)benzoic acid (92). Alternatively, dibenzylamine may similarly react to form 2-(dibenzylcarbamoyl)benzoic acid (93). In some embodiments, 92 is subsequently subject to Grubbs ruthenium-catalyzed ring closing metathesis condition to form 2-(2,5-dihydropyrrole-1-carbonyl)-benzoic acid (94). In some embodiments, 92 is first turned into esters before the ring closing metathesis reaction takes place. In some embodiments, the phthalic anhydride is replaced with maleic anhydride. In some embodiments, the phthalic anhydride is replaced with pyromellitic anhydride. In some embodiments, terephthalic acid may react with diallylamine under condensation conditions to form N,N,N′,N′-tetraallyl-teraphthalimide, which is subsequently subject to the Grubbs ring closing metathesis condition to form 1,4-bis(2,5-dihydropyrrol-1-carbonyl)benzene (96). In some embodiments, the phthalic anhydride is replaced with trimellitic anhydride. In some embodiments, the products described may be further subject to reductive condition to reduce carbonyl groups into —CH₂— using e.g. LiAlH₄, PMHS alone, PMHS in conjunction with [Fe₃(CO)₁₂] (described later), phenylsilane (2˜3 eq. per carbonyl designed to be reduced) catalyzed with 10 mol % of zinc acetate, silane catalyzed by Ni(dme)Cl₂, PMHS catalyzed by Co₂(CO)₈, or nickel acetate under alkaline condition. In some embodiments, having fewer carbonyl groups improves capacity and/or activity. However, the nature of the applications often mandates a low preparation cost. In some embodiments, having one carbonyl on each 5-member heterocycle provides a good balance between cost and capacity/activity.

In an embodiment, the OS may include 2,6-bis-(2-aminoethyl)-1,3,5,7-tetrahydropyrrolo[3,4-f]isoindole (18). In an embodiment, pyromellitic anhydride is reacted with excess amount of ethylene diamine to form 2,6-bis-(2-aminoethyl)-1,3,5,7-tetrahydropyrrolo[3,4-f]isoindole-1,3,5,7-tetraone (74), followed by reduction (such as those described above with respect to 10) to form 18. In an embodiment, 18 is reacted with two eq. of phthalic anhydride under condensation condition (e.g. in Dean-Stark apparatus to form 2,6-bis-(2-phthalimidoethyl)-1,3,5,7-tetrahydropyrrolo[3,4-f]isoindole (19). In an embodiment, 19 is further subject to a reducing reaction, similar to 10 to form 2,6-bis-(2-phthalimidoethyl)-1,3,5,7-tetrahydropyrrolo[3,4-f]isoindole (20). In some embodiments, 19 and 20 may be beneficial in that they provide an OS having a molecular weight of a medium range (between 400 and 600) that is not too small (which may lead to migration and leaching issues) or too large (which sometimes lead to incompatibility with base resin and cause incompatibility or immiscibility). In some embodiments, 19 may be partially reduced to 2,6-bis-(2-phthalimidinoethyl)-1,3,5,7-tetrahydropyrrolo[3,4-f]isoindole (45), e.g. by catalyzed hydrogenation reaction using common catalysts such as Pt or R₁₁ compounds. In some alternative embodiments, precursors 74 may first condense with, e.g. phthalic anhydride, to form 18, and subsequently reduced, either completely to 20, or partially into 45 (or similar molecules, referred to as 45′, having carbonyls on one to seven (out of the total eight) C₁ positions of 20). For example, 2,6-bis-(2-isoindolinoethyl)-pyrrolo[3,4-f]isoindole-1,3,5,7-tetraone is considered to be one example of 45′. In an embodiment, the OS may include 2. In some embodiments, trimellitic anhydride is reduced (similar to 10) to 5-hydroxymethylphthalide (21). In some embodiments, 21 is introduced into the PET manufacturing process as an end cap group (more details described below). In some embodiments, 21 is beneficial in that once it fulfills its functioning (e.g. having reacted with oxygen), it turns into a phthalic anhydride. The phthalic anhydride as an end group may react with end groups of PET chain to increase the molecular weight. By contrast, if the OS is not at the end of the PET chain, but rather in the middle of the chain, similar reaction will cause branching instead of linear increase in length. Such branching may be sometimes undesirable, for example for improving strength. In some embodiments, 18 may be replaced with 5-(N-hydroxyethyl-aminomethyl)-2-(2-hydroxyethyl)-isoindoline (95), or analogues having one or two of the 1,3,5 positions of the isoindoline ring substituted with ═O. In some embodiments, the phthalic anhydride is replaced with maleic anhydride.

In some embodiments, the OS may be 1,1,2,2-tetrakis(phthalimidino)ethane (97), for example, by reacting phthalide with 1,1,2,2-ethanetetraamine under heating conditions. In some embodiments, 97 may include substitutions on the benzene rings, for example, 5-hydroxy substitutions which can serve as the activator. 5-bromophthalide may be used as the starting materials. In some embodiments, 5-carboxyphthalide may first be converted into its ester, and subsequently reacted with 1,1,2,2-ethanetetraamine under similar conditions. Following removal of the ester protection, 5-carboxy-substituted 97 is received. In some embodiments, the 5-carboxy-groups may serve as tethering points to interact with base polymer, or serve as reaction sites in a comonomer or monomer in polyester synthesis, similar to embodiments described elsewhere in this application. In some embodiments, one, two, or three of the carbonyl groups of 97 may be reduced to methylene groups. In some embodiments, one, two, or three of the phthalide methylene groups may be oxidized to carbonyl groups. In some embodiments, the 1,1,2,2-ethanetetraamine may be replaced with any other tetramines, triamines, diamines, pentamines, hexamines, oligoamines, polyamines, polyetheramines, etc. In some embodiments, the OS may be 2,4,6-tris(5-carboxyphthalimidino)-1,3,5-triazine (99). In some embodiments, 99 may be synthesized by first derivatizing 5-carboxyphthalide with an alcohol to form ester. The product reacts with melamine at a 3.3:1 molar ratio in DMSO at about 100° C. overnight and then cooled down to room temperature. The solution is poured into ice water and hydrolyzed to form 99. In some embodiments, the triazine core provides UV-absorption and serve as an activator.

In some embodiments, the OS may be N,N′-bisallyl terephthalamide (46). This may be formed by reacting excess amount of allylamine with dimethylterephthalate at refluxing condition for 2 hours. For example, PET (which could be PET oligomer, PET polymer, modified PET, pure PET, PET with certain additives, virgin PET, recycled PET, PCR PET, waste PET bottles, waste PET fibers, etc.) is treated for 2 h with an excess of allylamine at 170° C. under pressure of 2 MPa to form the product 46. Ethylene glycol is received as a byproduct. In some embodiments, such allyl amide-based OS are less prone (e.g. than benzyl amide counterparts) to generate aromatic aldehydes or aromatic acids that have raised concern of safety considerations for food and beverage packaging applications. In some embodiments, there may be improved compatibility with base resins (such as non-PET resins, polyolefin base resins, etc.) due in part to chain flexibility (such as with linear polyester resins—such as those not including aromatic rings). In some embodiments, there may be improved compatibility with base resins due in part to structural similarities (such as with polyolefin base resins). In some embodiments, the allylic amide may have a low molecular weight, and thereby improves compatibility with polymeric base resins.

For another example, the OS may be benzylbenzoate (47). In some embodiments, benzaldehyde reacts in presence of a catalytic amount of aluminum isopropoxide under mild reaction conditions (e.g. 20° C. to 60° C.) to form benzyl benzoate via Tishchenko Reaction. In some embodiments, the OS may be a polymeric or oligomeric molecule having multiple 47 fragments. For example, benzaldehyde is replaced with terephthaldehyde or isophthaldehyde and the reaction proceeds under similar conditions. This may be useful for, for example, balancing between migrations of the OS, degradants thereof, and the appearance (such as haze) of packaging articles having the OS, embedded therein. In some embodiments, a mixture of benzaldehyde and terephthaldehyde and/or isophthaldehyde is used as the starting material. This allows the fine-tuning of the structure of the OS. In some embodiments, rather than aluminum isopropoxide, sodium benzylate is used as the catalyst for the above reactions. In some embodiments, 47 or the polymeric or oligomeric molecules having the 47 fragments described above may be further derivatized to form other OS. This allows the fine-tuning of the oxygen scavenging activity. For example, 47 may be reacted with benzylamine to form benzylbenzamide (48), or with dibenzylamine to form N,N-dibenzylbenzamide (49). Moreover, benzylbenzoate may be reacted with diamines or triamines to form molecules having similar 48 elementary structures but larger molecular weights. In some embodiments, the polymeric or oligomeric molecules including multiple 47 fragments may be treated with benzylamine to form polybenzylbenzamide, poly(N-benzyl-benzylbenzamide), polymers including both 47 and 48 moieties, or other similar products. In some embodiments, the polymeric or oligomeric molecules including multiple 48 fragments may similarly treated in presence of an exchange catalyst. In some embodiments, 47 is reacted with xylene-p-diamine to form a product having two benzylbenzamide fragment arranged in a para-configuration. The para-configuration may be particularly beneficial for applications involving PET when appearance (such as haze) is of particular importance. However, other configurations (such as meta-) may be more beneficial due to easier access to starting materials, lower costs, or other reasons. In some embodiments, the starting material may include terephththaldehyde or isophthalaldehyde in the first step, and xylenediamine or other diamines in the second derivatization step. Accordingly, the product may have multiple oxygen scavenging fragments. As described later, those multiple oxygen scavenging fragments may be beneficial for the oxygen scavenging activity. Moreover, in some embodiments, triamines, or polyamines may instead be used in the second step, to form a polymeric framework with multiple voids among branches of the structures. In some embodiments, such multiple voids may be beneficial for rapidly removing oxygen, e.g. in the headspace of a packaging article. In some embodiments, starting from structures with 48 or 49 moieties may be easier to form such multiple voids than starting from structures with only 47 moieties. In some embodiments, a mixture of terephththaldehyde, o-phthalaldehyde, and/or isophthaldehyde may be used in the first step to form polymer structures having different branching patterns to lead to an increased amount of voids and/or voids of varying sizes. Alternatively or additionally, a mixture of para-, meta- and/or ortho-xylenediamine may be used in the second step to lead to further increased amount of voids and/or voids of further varying sizes. However, in some circumstances, the presence of larger voids may increase the permeation rate of the packaging, where these structures may preferably be avoided.

In some embodiments, discrete, oligomeric, or polymeric molecules having 48 or 49 fragments are reduced such that the amide group (—(C═O)—N—) is reduced into (—CH₂N—). Accordingly, the product includes dibenzylamine (50) or tribenzylamine moieties. Any suitable method, such as catalytic hydrogenation with H₂, LiAlH₄ or other metal hydride-based reduction, hydrosilylation-based or borane-based reduction may be used. In some embodiments, PMHS or other silanes may be used. For example, the amide starting material (1.0 mmol) is charged into an oven-dried Schlenk tube and [Fe₃(CO)₁₂] (0.02-0.1 mmol). PMHS (4.0-8.0 mmol) and dry toluene or dibutylether (3 mL) is added respectively after purging the Schlenk tube with argon. The mixture is stirred at 100° C. for 1 day. The reaction mass is filtered through Celite and washed with each. The combined fractions are concentrated under reduced pressure and further purified by column chromatography. In some embodiments, the reduction is done in two steps. For example, PMHS may first react with the substrate, and [Fe₃(CO)₁₂] is subsequently added. The products formed are also OS, either discrete molecular, oligomeric, or polymeric, depending on the starting material. Similarly, discrete, oligomeric, or polymeric molecules having 47 fragments may be reduced to form dibenzylether moieties as OS.

For yet another example, the oxygen scavenger may include a benzyl urea moiety. In some embodiments, the oxygen scavenger may be N,N′-dibenzylurea. In some embodiments, the oxygen scavenger may be a xyleneurea oligomer or polymer. In some embodiments, the oxygen scavenger may be asymmetric. For example, the oxygen scavenger may include N-benzylurea. For example, benzylamine (2 mmol) is mixed with 1 N aqueous HCl (3 mL) and stirred. Potassium cyanate (4.4 mmol; 2.2 equiv.) is added. The reaction mixture is maintained at room temperature for 6 hours with stirring. Upon completion of the reaction as indicated by TLC, the reaction mixture is diluted with 1 N aqueous HCl (3 mL) and extracted with methylene chloride (20 mL). The organic layer is washed with water (5 mL), dried over anhydrous sodium sulphate and concentrated under reduced pressure to afford N-benzylurea. In some embodiments, the oxygen scavenger may include 4-(ureidomethyl) benzoic acid. It may be synthesized based on the procedure above for N-benzylurea, except that the starting material uses 4-(aminomethyl)benzoic acid rather than benzylamine, and the reaction time is extended from 6 hours to about 12 hours. In some embodiments, these asymmetric OS may have the benefit of being easily further derivatized (such as to include additional functional groups, to improve scavenging activities—such as by incorporating activator moieties described later, or to incorporate additional functionalities); while the symmetric scavengers may be easier to synthesis and/or easier to form polymeric molecules therefrom.

In some embodiments, the oxygen scavenger may include imine functional groups, which is also often referred to as Schiff Bases. For example, the oxygen scavenger may be N-benzylidenebenzylamine (51), N,N′-dibenzylideneethylenediamine (CAS #104-71-2) (52). In some embodiments, the oxygen scavenger may include salen-like motife. For example, bis(acetylacetone) xylylenediamine (53) may be synthesized from condensation between two moles of acetylacetone and one mole of xylylenediamine in stoichiometric ratio under Dean-Stark conditions.

In one aspect, the OS may include a C₁ carbon atom bearing at least one hydrogen (referred hereinafter as “H₁”), and the carbon atom C₁ is further directly attached to (1) a carbonyl group and (2) either a heteroatom or a conjugated group. For example, the carbonyl group may be that of an ester group or that of an amide group. For example, the heteroatom may be an oxygen atom or a nitrogen atom. For example, the conjugated group may be a C═C double bond or an aromatic ring (e.g. benzene).

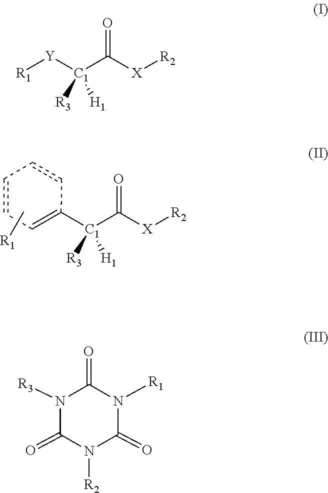

In some embodiments, the OS has the general formula (I). For example, the OS may be glycine, a glycine anhydride, a glycine derivative, where X is oxygen (O), and Y is —NR—. R may be any suitable organic or organometallic radicals. For example, it may include an amine functionality directly attached to the C atom, and further includes a carboxylic group directly attached to the C atom through the carboxylic ketone carbon. Glycine is commercial available. Synthesis of molecules incorporating the glycine fragment is well documented, such as in drug synthesis and protein analysis literature. In some embodiments, R₁ group includes a conjugated group, such as a benzene directly attached to the glycine amino group. For example, the OS may include N-phenylglycine (CAS No.: 103-01-5), or N-phenylglycine esters (e.g. N-phenylglycine ethyl ester, CAS 2216-92-4). In some embodiments, the OS may be a cyclic N-phenylglycine dimer (anhydride). In some embodiments, the phenyl ring of the N-phenylglycine is further derivatized with an electron donating group, such as methoxy group, ethoxy group, amino group, or the like, directly attached to the p-position of the phenyl ring relative to the glycine amino group. For example, the OS may be 2-(4-methoxyanilino)acetic acid (CAS No 22094-69-5) or esters thereof. For example, the ester version, ethyl 4-methoxyphenylaminoacetate, may be synthesized by reacting p-anisidine (1 eq.), ethyl chloroacetate (1 eq.) and anhydrous potassium carbonate (1.5 eq.) in dry acetone under refluxing condition for 20 h. The reaction mass is filtered, and the filtrate is concentrated and then poured onto ice-cold water with rigorous stirring. The organic layer is then extracted with diethyl ether. The ether layer is washed with 5% hydrochloric acid, dried over anhydrous sodium sulfate, and evaporated to form the product. In some embodiments, the OS is a polymer having the derivatized glycine as a monomer unit, a comonomer unit, or a capping unit.

In some embodiments, the OS is a glycolamide derivative, wherein X is —NR—, and Y is O. For example, it may include a hydroxy functionality directly attached to the C₁ atom, and further includes a carboxylic amide (R′R″—N—C(═O)—) group directly attached to the C₁ atom through the amide ketone carbon. Glycolamide is commercial available. Synthesis of molecules incorporating the glycolamide fragment is also well documented, such as in drug synthesis and protein analysis literature. In some embodiments, R₁ may include a carbonyl, such that the glycolamide is an ester. For example, R₁ may be a benzoyl group, and the OS may be carbamoylmethyl benzoate. In some embodiments, benzoic acid (0.01 mol) is dissolved in ethyl acetate (40 mL). Triethylamine (0.011 mol), sodium iodide (0.001 mol), and 2-chloroacetamide (0.011 mol) are added. The mixture is heated for 3 hours at refluxing condition. The reaction mass is cooled and filtered. The filtrate is washed with sodium thiosulfate aqueous solution (2%), sodium bicarbonate aqueous solution (2%), and water. The liquid is further dried over anhydrous sodium sulfate, and the solvent removed under vacuum. Recrystallization is conducted. Derivatized benzoic acid and derivatized 2-chloroacetamide may alternatively used under similar conditions to form other derivatives of glycolamide. In some embodiments, the amide nitrogen is further attached to a conjugated system, such as phenyl group. For example, the OS may be 2-benzoyloxy-N-(4-methanesulfonylamino-3-phenoxy-phenyl)-acetamide. In some embodiments, 2-Chloro-N-(4-methanesulfonylamino-3-phenoxy-phenyl)-acetamide is first synthesized. For example, 4-methanesulfonylamino-3-phenoxy aniline (3.6 mmol) is taken mixed with chloroform and 0.6 mL of triethylamine. This mixture is stirred and cooled to 0° C. using an ice bath. 1 eq. of alpha-chloroacetyl chloride is then added dropwise, and the mixture is then cooled to room temperature while maintaining stirring for another half an hour. Upon completion of the reaction as indicated by TLC, the mixture is diluted with cold water and extracted with chloroform, dried, and purified by recrystallization. The product is then mixed with 1 eq. of benzoic acid, 0.1 eq. of potassium iodide, 1.1 eq. of triethylamine in N,N-dimethylformamide and stirred for about 30 min at 90° C. The reaction mixture is then poured into water and extracted with ethyl acetate. The organic phase is washed, dried and purified to afford the 2-benzoyloxy-N-(4-methanesulfonylamino-3-phenoxy-phenyl)-acetamide. In some embodiments, the OS is a polymer having the derivatized glycolamide as a monomer unit, a comonomer unit, or a capping unit.

In some embodiments, the OS is a glycinamide derivative, where X and Y are both —NR—. For example, it may include an amino functionality directly attached to the C₁ atom, and further includes a carboxylic amide group directly attached to the C₁ atom through the amide ketone moiety. Glycinamide is commercial available. Synthesis of molecules incorporating the Glycinamide fragment is well documented, such as in drug synthesis and protein analysis literature. In some embodiments, R₁ group includes a conjugated group, such as a benzene directly attached to the glycine amino group. For example, the OS may include N-phenyl-2-(phenylamino)acetamide (CAS 2567-62-6). In some embodiments, one or both of the phenyl rings of N-phenyl-2-(phenylamino)acetamide is further derivatized with an electron donating group, such as methoxy group, ethoxy group, amino group, or the like, directly attached to the p-position of the phenyl ring relative to the glycinamide moiety. For example, N-phenyl-2-(4-methoxyphenylamino)acetamide may be the OS. In some embodiments, ethyl chloroacetate is used to react with benzylamine in presence of potassium carbonate at 0° C. for a time duration of about 6 hours to overnight to first form 2-Chloro-N-phenylacetamide. The product is then reacted with p-anisidine in presence of sodium acetate in ethanol under refluxing condition to form the N-phenyl-2-(4-methoxyphenylamino)acetamide. Other derivatives may alternatively be synthesized as OS. In some embodiments, the OS is a polymer having the derivatized glycinamide as a monomer, a comonomer unit, or a capping unit.

In some embodiments, the OS is a glycolic acid (or glycolate) derivative, where both X and Y are O. For example, it may include a hydroxy functionality directly attached to the C₁ atom, and further includes a carboxylic group directly attached to the C₁ atom through the carboxylic ketone carbon. Glycolic acid is commercial available. Synthesis of molecules incorporating the glycolic fragment is well documented, such as in drug synthesis and protein analysis literature. Moreover, glycolic acid may be derived from sugarcane (in other words, sustainable resources, or waste biomass). In some embodiments, the OS may be poly(glycolic acid). In some embodiments, the C₁ atom may be further derivatized. For example, the C₁ atom is further directly attached to a phenyl ring. The phenyl ring improves the oxygen scavenging reactivity. In other words, the OS includes a mandelic acid or derivatives thereof. In some embodiments, the OS is a cyclic mandelide dimer (anhydride), mandelide oligomer, or a poly(mandelic acid).

As described above, the OS may be a polymer incorporating the above-described OS fragments. In some embodiments, an alpha-hydroxy acid (such as mandelic acid, lactic acid, and glycolic acid) is reacted with excess ethylene glycol (or other diols), thereby forming a derivatized diol including the OS fragment. Alternatively, the alpha-hydroxy acid is reacted with excess amount of diamines to form a derivatized diamine including the OS fragment. These diols and diamines may react with terephthalic acid (or any other diacids, such as oxalic acid, malonic acid, succinic acid, glutaric acid, adipic acid, pimelic acid, any other dicarboxylic acid, or combinations thereof) to form polyesters (e.g. PET) or polyamides, respectively, that includes the above-described OS. These polymers are OS. Glycine-based OS fragments may be similarly incorporated into polymer backbones, and glycolamide-based and glycinamide-based may also be similarly incorporated in presence of an exchange catalyst.

In some embodiments, the OS includes a conjugated group (such as an aromatic ring or a double bond) directly attached to the C₁ atom, and further includes a carboxyl group or carboxylic amide group directly attached to the C₁ atom through the carboxyl ketone carbon. In other words, it may have the above formula (II). For example, X may be oxygen, R₁ may be H, R₂ may be methyl, and the OS may be methylphenylacetate. For example, X may be —NH—, R₂ may be phenyl, and the OS may be N-phenylphenylacetamide. In some embodiments, phenylacetic acid (1.0 eq) reacts with aniline (1.2 eq) with 10 mol % B(OH)₃ in toluene (0.5 L) reflux for 10 h under Dean-Stark condition to form phenyl phenylacetate. In some embodiments, X may be —NH—, R₁ may 4-hydroxyphenol, R₂ may be 3,5-dimethylphenyl, the OS may be N-(3,5-dimethylphenyl)-2-(4-Hydroxyphenyl)acetamide. In some embodiments, it may be synthesized as follows: 3,5-Dimethylaniline (1.2 eq) is reacted with 4-Hydroxyphenylacetic acid (1.0 eq) in presence of boric acid B(OH)₃ (10 mol %) in toluene (1 L for 80 g of 4-Hydroxyphenylacetic acid) under refluxing condition in a Dean-Stark setup. At the end of the reaction, the product is purified by filtration and recrystallization. Without being limited by theory, the presence of the phenol group improves the efficacy of this OS. In some embodiments, the synthesis of the OS may use any other methods, for example, this OS is an intermediate for the synthesis of the drug efaproxiral, and can employ any of those methods reported in the relevant literature. Moreover, the drug efaproxiral is also an OS contemplated by this present disclosure. In some embodiments, the OS is a polymer having the, for example, phenylacetate or phenylacetamide, as monomer units or comonomer units.

In some embodiments, the OS may be based on isocyanuric acid or cyanuric acid, and may have the general formula (III) above. For example, the R₁, R₂, and R₃ may each be an allyl group, and the OS may be triallyl isocyanurate. For example, the R₁, R₂, and R₃ may each be a benzyl group, and the OS may be tribenzyl isocyanurate. For example, R₁, R₂, and R₃ may be a polyalkylene chain, and the OS may be a polyalkylene glycol (or polyetherol)-derivatized isocyanuric acid. For example, the OS may be a polyethylene glycol containing 1,3,5-triazine-2,4,6(1H,3H,5H)-trione rings. In some embodiments, isocyanuric acid (0.1 mol) is placed together with ethylene carbonate (0.33 mol) and 0.4 g of potassium carbonate catalyst. The mixture is heated to 155-160° C. and kept at this temperature for about 6 h. The solid portion is received as the product (1,3,5-tris-polyethyleneglycol-1,3,5-triazine-2,4,6(1H,3H,5H)-trione) upon work up. The product is an OS. Moreover, this oxygens scavenger includes a triazine core that is sensitive to a particular wavelength of UV-light. In some embodiments, this product is used as an UV-activated or UV-accelerated OS, where an article including the product is subjected to UV-light in order to initiate or accelerate the oxygen scavenging reaction. This may be particularly useful in applications where induction period is problematic, such as where the contents react with trace amount of oxygen rapidly. This may also be applied as a UV-blocker to simultaneously protect UV-sensitive contents from UV-exposure. In some embodiments, this product may be used towards meat packaging, such as packaging for pork, beef, etc. In some embodiments, instead of polyalkylene glycol, other OS moieties may be similarly incorporated onto the cyanuric acid platform to form other OS. In some embodiments, the OS including the cyanuric acid motif may be synthesized using nucleophilic aromatic substitution of cyanuric chloride by, for example, OS precursors having an amine functionality. In some embodiments, different OS moieties (e.g. 2 or 3) may be attached to the same triazine core by utilizing sequential reactions with the amine derivatives of the oxygen scavenging moieties with sequentially increased reaction temperature. For example, a first OS moiety replaces a first chlorine atom of the cyanuric chloride at 0° C. to form an intermediate; a second chlorine atom of the intermediate is further replaced by a second OS moiety at a second temperature (such as room temperature). While these examples illustrate a situation R₁, R₂, and R₃ are the same organic fragment, they may alternatively be all different, or one of them being different from the other two. In some embodiments, the different OS moieties may be responsible for different application scenarios. In some embodiments, a first such OS moiety may be responsible to serve as RS during the processing of the packaging article (as described in detail below), while the second such OS moiety may be responsible to rapidly absorb residual oxygen in the headspace of the packaging. In such embodiments, the second OS moiety may be configured to be much more active (e.g. having substantially lower BDE) than the first OS. In some embodiments, a third such OS moiety may be responsible to serve to protect against ongoing permeation of oxygen from outside atmosphere. In such embodiments, the third OS moiety may have an activity level between that of the first and the second OS moieties. In some embodiments, at least one of R₁, R₂, and R₃ includes a tethering group for interaction with polymer backbones.

In some embodiments, the OS molecule may include an activator moiety (or interchangeably referred to as “activator group” or “activator”) conjugatively connected (directly or indirectly) to the C₁ atom. The terms atom A being “conjugatively connected,” “conjugatively attached” or “conjugatively bonded” to atom B is defined as both A and B being directly connected, directly attached, or directly bonded to a same conjugation system (such as aromatic groups, double bonds, triple bonds, as well as hyperconjugation of lone pairs) between each other. The term “directly” means the connection to the conjugation system is through a single bond or that the relevant atom is physically immediately adjacent to the conjugation system. For example, the two benzylic carbons of p-xylene, o-xylene, and m-xylene are each conjugatively connected to each other. Because those two benzylic carbons are both directly attached to the same π-system of the benzene ring. Moreover, the two benzylic carbon of 4,4′-Dimethylbiphenyl is also conjugatively connected, because the extensive conjugation between the two benzene rings provides a same and large conjugation system. Furthermore, the two benzyl carbons of 4,4′-dimethylbenzophenone and 4-methylphenyl 4-methylbenzoate are also conjugatively attached to each other. Additionally, the three carbon atoms of trimethylamine are also each conjugatively attached to each other via the lone pair (which is construed to be a conjugation system here) of the central N. However, the four peripheral carbon atoms of neopentane is not conjugatively attached to the center carbon or to each other, as the center carbon does not have conjugation or lone pair that allows electronic delocalization, and no resonance structure may be written for it. It is noted that the term A being “conjugatively connected” to B does not require A being directly connected (that is, directly bonded) to B. But it does require both A and B being directly connected to a same conjugation system. When A and B are conjugatively attached to each other, they may either be conjugatively coupled to each other or not be conjugatively coupled. The term atom A being “conjugatively coupled” to atom B refers to the situation a radical formed on atom A may be interconverted with a radical formed on atom B using resonance structures. For example, the two benzylic carbons of a p-xylene and an o-xylene are conjugatively coupled to each other, although the two benzylic carbons of a m-xylene are not conjugatively coupled to each other. Moreover, the three carbon atoms of trimethylamine are conjugatively coupled to each other. In some embodiments, having an activator conjugatively attached to C₁ improves the OS reactivity at the C₁ site. Moreover, having an activator conjugatively coupled to C₁ further improves the OS reactivity at the C₁ site. Depending on specific application scenarios, in some embodiments, having an activator conjugatively attached to C₁ may sufficiently improve the C₁ activity towards oxygen to the designed level, e.g. less OS catalysts (such as cobalt or manganese compounds) may be required for the functionality, which improves recycling stream compatibility. In some embodiments, the magnitude of activity may require having an activator conjugatively coupled to C₁. For example, a catalyst-free OS application for the ambient condition (e.g. without oxidation catalysts such as cobalt/manganese compounds) may require an activator group conjugatively coupled to the C₁ atom in some circumstances. In an embodiment, the OS is used in PET without the presence of an oxygen scavenging catalyst. The atom of the activator moiety directly attached to the conjugation system is referred to as “activated atom.”

In some embodiments, the activator moiety may be a radical generator at the respective application condition (such as the lightly frozen condition, the refrigerated condition, the room temperature condition, the warm condition, etc.). For example, the activator moiety may be the O—H moiety of a phenol, the O—H moiety attached to a non-benzene conjugated system (such as ascorbic acid), the O—H of the N-hydroxyphthalimide, the O—H of the N-hydroxybenzotriazole, or the O—H moiety directly attached to a nitrogen of a conjugated system. In these examples, the O of the O—H moiety is the activated atom. For another example, the N—H moiety of an aniline moiety, the N—H moiety directly attached to a C═C double bond, the N—H moiety of an amide group having the N—H moiety directly attached to a C═C double bond or directly attached to an aromatic ring (such as anilide) may also be the activator moiety, where the N of the N—H moiety is the activated atom. In some embodiments, these described O—H and N—H groups may be hindered by surrounding organic groups (such as sterically hindered phenols or amines) to improve the performance of the activator.

In some embodiments, the OS may have formula (IV-1).

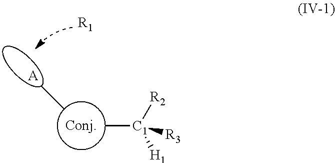

where the circle represents a conjugation system, the oval represents R₁ group having an activator as described below. Formula (IV-1) illustrates the activator R₁ being conjugatively attached to C₁. Although not specifically illustrated in formula (IV-1), depending on the relative substitution positions, the activator R₁ may also be conjugatively coupled to C₁. The R₁ includes an activated atom A which can be O, N, C, etc. The R₁ is the activator and may be selected from one of the following moieties, where the wavy bond represents the dangling radical connected to the rest of the formula:

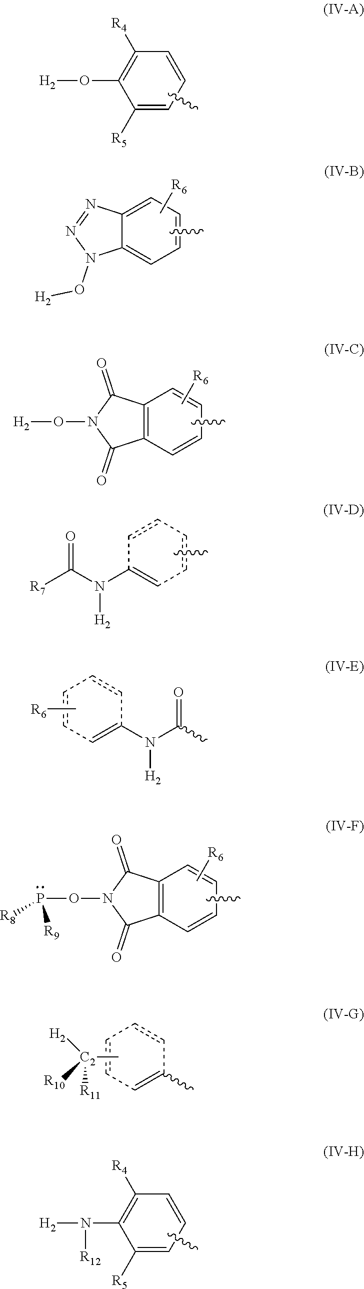

where R₄-R₁₂ may each be selected independently from a hydrogen, an organic residue, and an organometallic residue. In some embodiments, R₄ or R₅ may be sterically bulky groups or hindering groups. In some embodiments, R₄ or R₅ may be a thio group (e.g. methylthio group —SCH3). In some embodiments, the presence of one or more such bulky groups improves the effectiveness of the activator. In some embodiments, the activating strength (or effectiveness) of the activator groups are correlated with the BDE of the weakest bond on the activator. The lower the BDE is, the more effective the OS would be. For example, phenol, BHT, and butylated hydroxyanisole (BHA) may each serve as the activator for a respective C₁ site. However, BHT and BHA are more effective than phenol in such functions, such that OS incorporating BHT or BHA activator groups are more active than OS incorporating simple phenol activator groups. In some embodiments, as the activator includes a conjugated portion itself, the circle representing the conjugated system of formula (IV) may be omitted. In other words, the activator may directly attach to the C atom.

In some embodiments, the activator may have the structure of formula (IV-A). For example, the activator may be a phenol O—H moiety. For example, the OS may be 2-benzyl-5-hydroxyisoindoline-1,3-dione (54), 4-hydroxy-2-indanone (55), 2-[3,5-di(tert-butyl)-4-hydroxybenzyl]isoindoline-1,3-dione (56) or 2-[3,5-di(tert-butyl)-4-hydroxybenzyl]isoindoline (57). In one embodiment, the OS includes 4,8-dihydroxy-2,6-bis-(2-hydroxy-ethyl)-1,3,5,7-tetrahydropyrrolo[3,4-f]isoindole (12). For example, a 1 L round-bottom flask is charged with durene (30 g, 223.5 mmol) and CCl₄ (500 mL). After stirring for 15 minutes to allow complete dissolution of durene, Bra (91.62 mL, 1788 mmol) is added to the reaction solution over 0.5 h. After the addition is complete, the reaction mixture is stirred at 54° C. for 24 h. The heating source is then switched from oil bath to lamp (GE Halogen, 100 w) and the reaction is refluxed for another 24 h. After cooling to r.t., the solids are filtered, washed with CCl₄ and dried to give 1,4-dibromo-2,3,5,6-tetrakis(bromomethyl)benzene (intermediate 1) as a white solid. A 500 mL round bottom flask is charged with intermediate 1 (5 g, 8.23 mmol), 65% nitric acid (250 mL) and 1 g sodium metavanadate. After refluxing for 12 h, the reaction mixture is concentrated under reduced pressure. Fresh 65% nitric acid (250 mL) is added to the residue and the reaction mixture is again refluxed for 12 h and concentrated under reduced pressure. The reaction mixture is repeatedly (3×) diluted with D.I. water (250 mL) and concentrated each time under reduced pressure. D.I. water (250 mL) is then added and the yellow solution is extracted with ether (3×100 mL). The ether layers are combined and concentrated under reduced pressure to a colorless solid, 3,6-dibromobenzene-1,2,4,5-tetracarboxylic acid (intermediate 2) which is used for the next step without further purification. A sublimation apparatus with water-cooled cold-finger is charged with crude intermediate 2 (2.72 g, 6.60 mmol). After heating to 205° C. at 10⁻² mm Hg for 24 h, a yellow solid is collected as 3,6-dibromobenzene-1,2,4,5-tetracarboxylic dianhydride (intermediate 3) and used for the next step without further purification. To a Schlenk flask is added intermediate 3 (3.00 g, 7.98 mmol), glacial acetic acid (30 mL) and monoethanolamine (15.96 mmol). The flask is fitted with a water-cooled condenser and the contents are refluxed with stirring for 8 h. The crude reaction mixture is concentrated under reduced pressure, methanol (50 mL) is added and the precipitate is collected by filtration. The crude product is purified by flash chromatography (silica gel, dichloromethane:hexanes 1:1) to give a colorless solid as intermediate 4. The intermediate 4 is converted to 12 following published method of CN101258139A. 12 includes two phenol groups which not only function as two activator groups, but also present synergistic effects to vastly improve activity. In an embodiment, a similar compound with only one phenol group may be formed using similar methods (where the first bromination step is adapted to derivatize only one of the two free benzene carbons). Although at less efficacy than 12, it may still function better than many conventional OS due to the presence of one activator group. It may also be a very efficient RS. In an embodiment, PET-12 may be prepared using similar methods as those of PET-11. In an embodiment, phenol groups of 12 may be first derivatized with a silyl group (e.g. trimethylsilyl) prior to the condensation reactions, and subsequently deprotected using conventional methods.

In some embodiments, the activator may have the structure of formula (IV-H). For example, the activator may include an aniline analogue of the phenol moiety described above with respect to formula (IV-A). In some embodiments, the activator may have the structure of formula (IV-C). For example, the activator may be the O—H of the N-hydroxyphthalimide. For example, the OS may be 4-benzylaminocarbonyl-N-hydroxyphthalimide (58). In some embodiments, trimellitic anhydride (40 mmol) and N-hydroxylamine.hydrochloride salt (44 mmol) is suspended in pyridine (50 mL) and stirred magnetically at 90° C. for about 15 h. At the end of the reaction, the reaction is quenched by addition of water (50 mL), and cooled to room temperature. The pH value is adjusted to 2-3 with HCl to achieve the 4-carboxy-N-hydroxyphthalimide as a white solid precipitation. The 4-carboxy-N-hydroxyphthalimide is reacted with benzylamine in p-xylene under refluxing condition for overnight to form 4-benzylcarbomoyl-N-hydroxyphthahlimide (58). In some embodiments, the activator may have the structure of formula (IV-E). For example, the activator may be a N—H moiety directly attached to a first conjugated group and further being directly attached to or adjacent to a second conjugated group. For example, the activator may be N—H directly attached to a benzene ring and to a carbonyl. For example, the activator may be the N—H of an N-phenylurea moiety. Alternatively, the activator may be the N—H of an isoproturon moiety. For example, the OS may be N-benzyl-N′-phenylurea (59). In some embodiments, NaN₃ (0.477 mmol) is added to a solution of benzylbromide (0.318 mmol) in acetonitrile (1.5 mL). The mixture is irradiated by microwave for 3 hat 95° C. (average power 240 W) under N₂ (2 bar) and magnetic stirring. After the reaction, the mixture is cooled to room temperature, filtered on paper. Polymer-bound diphenylphosphine (PS-PPh₂) (0.477 mmol) and aniline (0.636 mmol) are sequentially added. The mixture is irradiated by microwave for 1.5 h at 50° C. (average power 70 W) and 3 h at 70° C. (average power 200 W) under CO₂ (14.5 bar) and magnetic stirring. Then, the mixture is filtered on a cartridge to remove the polymer-bound diphenylphosphine oxide. The solvent is then evaporated under vacuum, the residue is dissolved in MeOH and Dowex® 50WX8-200 is added. The mixture is stirred at room temperature for 15 min. Finally, the mixture is filtered on paper and the solvent is evaporated under vacuum to provide 59.

In some embodiments, the activator may have the structure of formula (IV-F). For example, the activator may be O-P bond of a phenolphosphite (or phenylphosphite). For example, the OS may be tris(2,6-di-tert-butyl-4-(benzoyloxymethyl)phenol)phosphite (60). In some embodiments, 2,6-di-tert-butyl-4-hydroxymethylphenol (CAS #88-26-6) undergoes condensation reaction with benzoic acid to form (3,5-di-tert-butyl-4-hydroxy)phenylmethyl benzoate intermediate. The intermediate then react with phosphorous trichloride PCl₃ in chlorobenzene (30 g) under reflux condition under nitrogen atmosphere with stirring maintained for 24 hours. The reaction mass is then concentrated, cooled and poured over ethyl acetate. The mixture is then washed and dried to afford 60. In some embodiments, the activator has the structure of formula (IV-G). For example, the activator may be a C—H moiety directly attached a first conjugated group and further being directly attached to or adjacent to a second conjugated group. For example, the activator may be a C—H, where the C of the C—H (that is the activated atom) is designated as C₂. Although C₂ is illustrated as being directly connected to the benzene ring, it may also be directly connected to the double bond on the wavy bond. C₂ may be directly attached to a double bond or an aromatic ring. In some embodiments, C₂ may be directly attached to two C═C double bonds, directly attached to two benzene rings, or directly attached to one double bond and one benzene ring. In some embodiments, R₁ may include multiple double bonds. In other words, R₁₀ or R₁₁ includes one or more double bonds. For example, the activator may be the allylic C—H of an oligo(1,3-butadiene) moiety or a poly(1,3-butadiene) chain conjugatively coupled to the C₁.

In some embodiments, the activator may not necessarily be conjugatively attached to the C₁ atom. Instead, the activator (for example, the C₂ described above) may be in proximity of the C₁ atom (such as within two C—C bond distance from the C₁, point-to-point in space). In other words, the OS has formula (IV-2), where the C₁˜A point-to-point distance in space represented by the double-headed arrow is within two C—C bond distances. The curved line of formula (IV-2) represents any saturated or unsaturated linkage. For example, the activator may be an allylic C—H tethered onto a benzene ring via a carbon chain (saturated or unsaturated, derivatized or underivatized). The carbon chain positions C₂ (which is the activated atom A in this case) within two C—C bond distance (point-to-point in space) away from the C₁ atom.

Still alternatively, the activator may not be in proximity of the C₁ atom, but the activator is conjugatively attached to an A-H moiety that is in proximity of the C₁ atom (such as within two C—C bond distance from the C₁). In some embodiments, the OS may be represented by the formula (IV-3), where A′ represent a secondary activated atom in proximity to C₁, as described later. For example, the activator may be conjugatively attached (such as conjugatively coupled) to an A′ atom that is in proximity of the C₁ atom (such as A′ being within two C—C bond distance from the C₁ point-to-point in space). A′ may be part of a bond (e.g. A′-H bond, PINO-A′ bond where A′ is attached to the oxygen of the PINO, etc.) that has a bond dissociation energy less than 87.5 kcal/mol (referred to as a labile bond).

These above situations, such as the activator being conjugatively attached to a C₁ atom, in proximity of the C₁ atom, and being conjugatively attached to an A′ atom of a labile bond that is in proximity with the C₁ atom, are collectively referred to as being “activating” the C₁ atom. In some embodiments, the C₁ atom of the OS is part of a C—O bond. Moreover, the O of the C—O bond is part of a conjugated system greater than a benzene ring. For example, the C—O bond may be that of a phthalimide-N-oxyl (PINO) radical. In other words, the C₁ atom is directly attached to the dangling oxygen of the PINO radical. In some embodiments, the OS may be N-(propargyloxy)phthalimide (66). In some embodiments, the OS may be N-(3,4,5-trihydroxyphenylmethoxy)phthalimide (67). In some embodiments, the OS may be N,N′-bis(3,4,5-trihydroxyphenylmethoxy) pyromellitic diimide (or alternatively, 2,6-di(3,4,5-trihydroxyphenylmethoxy)-pyrrolo[3,4-f]isoindole-1,3,5,7-tetraone) (68). In some embodiments, the OS may be N-benzyloxyphthalimide (69). For example, N-benzyloxyphthalimide may be synthesized by condensation reaction of N-hydroxyphthalimide with benzyl chloride at 120° C. for about 3 hours, using triethylamine to remove hydrochloride byproduct. Derivatized molecules may be similarly synthesized.

Still alternatively, the activator may include a carbon atom C₃ on its conjugation system, where such carbon atom is attached to R₂ and H₁, as represented by the formula (IV-4):

Where the conjugation system includes a ring structure, the carbon atom is not on the ring structure itself, but rather on a double bond coupled to the conjugation system. In some embodiments, R₂ may be another conjugation system, such as a benzene ring, a double bond, a mesomeric group, such as an amine group, a hydroxy group, an ether group, a carbonyl group, an imine group, a thioether group, or the like. In some embodiments, R₂ may be hydrogen. For example, the OS may be 3,5-di-tert-butyl-4-hydroxy-styrene (70). In some embodiments, R₂ may be carboxyl group attached to the carbon atom with the carbonyl carbon. For example, the OS may be trans-3,5-di-tert-butyl-4-hydroxy-cinnamic acid (71). In some embodiments, 71 is synthesized from oxidation of 3,5-di-tert-butyl-4-hydroxy-toluene to the aldehyde and condensation under Knoevenagel condition or Doebner condition. In some embodiments, 70 may be synthesized from decarboxylation of 71 in dimethyl sulfoxide at 130° C. for half an hour.

In some embodiments, the OS may include a C₁ atom conjugatively coupled to one fragment that includes multiple activators, such as those described above. For example, the OS may be 3,4-dihydro-N-(3,4-dihydroxyphenyl) isoquinoline-2(1H)-carboxamide (61). This molecule has two O—H moieties of a phenol, and a N—H moiety of an aniline adjacent a urea carbonyl. In some embodiments, 1,2,3,4-tetrahydroisoquinoline (10 mmol) is dissolved in water and cooled to 5° C. 3,4-dimethoxyphenyl isocyanate (10 mmol) is slowly added in such way that the temperature of reaction mixture is maintained at about 5° C. The reaction mixture is stirred for about 30 minutes, and the product precipitates out. When TLC indicates the reaction is complete, the solid is collected as 3,4-dihydro-N-(3,4-dimethoxyphenyl)-2(1H)-isoquinolinecarboxamide. The product is then suspended in water and sealed in a Parr bomb. After being heated at 365° C. for 24 hours, the reaction mixture is cooled, concentrated and worked up to form 61. In some embodiments, the OS is N-(3,4,5-trihydroxyphenylmethyl) benzamide (62). For example, ethyl benzoate is reacted with excess amount of 3,4,5-trihydroxybenzyl amine at room temperature to form the product. In some embodiments, the OS is N,N′-bis(3,4,5-trihydroxybenzyl) terephthalamide (63). For example, instead of ethyl benzoate, diethylterephthalate is used as the starting material. In some embodiments, the OS may include one C₁ atom conjugatively connected to multiple fragments, each of them being activators. In some embodiments, the OS is N,N′-xylylenebis(salicylimine) (64). For example, salicylaldehyde is condensed with p-xylylenediamine in 1:1 stoichiometric ration in a Dean-Stark set up to remove byproduct water to form the product. The product is an OS that includes the O—H activator moiety. As compared to another OS, such as N,N′-dibenzylidenexylylenediamine (65) (which may be synthesized similarly using benzaldehyde rather than salicylaldehyde as a starting material), 64 may have better reactivity. Likewise, in some embodiments, the OS may include a C₃ atom being part of a conjugation system that includes multiple activators, such as those described above.

It should be noted that many of these listed activator moieties are commonly considered to be antioxidants and thermal stabilizers. In other words, they are RS without the presence of C₁ or C₃ active sites. However, as described above, not all antioxidants or thermal stabilizers are OS. For example, some activator moieties when used as a standalone molecule without additional active sites may only function as RS but not always function as an OS. For example, butylated hydroxytoluene (BHT) includes the structure (IV-A) but is not an active OS. However, some activators (such as those of structure (IV-G) may also function as an OS itself. Nevertheless, the interaction between C₁ (or C₃) and C₂ further improves the OS activity. In some embodiments, the activator exists as standalone molecules but coexist with one or more of the OS described herein or in the '209 application. In such embodiments, the concentration of the activators and the concentrations of the OS may need to be adjusted to achieve the desired activity. For example, a detailed ladder to find the optimal concentration for the activator molecule is required. This is similar to the method described in the '209 application for the determination of proper catalyst concentration.

In an embodiment, the activator may be a UV-activator, such as having formula (III) above. It is noted that several examples above with respect to formula (III) describes the C₁ active site being directly attached to the nitrogen of the triazine core. In such examples, the triazine core not only serves as the UV-activator, but also as the part of the chemical environment that establishes the reactivity of the C₁ active site. In some other embodiments, the C₁ active site may not be directly attached to the triazine core. In such examples, the triazine core may only serve as the UV-activator, and the C₁ activity may be established by the effects of other groups attached thereon.

In an embodiment, the concept of the above example may be applied to other OS. Particularly, any other OS described with respect to PCT/US20/15209 or U.S. 63/056,667 may similarly be derivatized to provide better RS activity (as thermal stabilizers), or better OS activity thereby reducing the amount of oxygen scavenging catalyst required. In some embodiments, such derivatized OS are used without any oxygen scavenging catalysts in oxygen scavenging applications (e.g. shelf-life extension). Moreover, although the above disclosure describe various examples with benzyl-type C₁ atoms, other types of C atoms described therein may similarly be adopted and/or derivatized without departing from the spirit of the disclosure. Other OS described elsewhere may similarly be improved using methods described herein as well. For example, the OS may be N,N′-[5-hydroxy-1,3-phenylenebis(methylene)] bis(isoindolin-1-one) (13) 5-bromophthalide and m-xylylenediamine may be condensed in a Dean-Stark apparatus in neat reaction condition to form N,N′-[5-bromo-1,3-phenylenebis(methylene)] bis(isoindolin-1-one), which may he convened to 13 using a conventional hydrolysis method, or using the method for intermediate 4 of 12 described above. In an embodiment, 13 may function as an RS with improved efficacy than N,N′-[1,3-phenylenebis(methylene)]bis(isoindolin-1-one); and function as an OS with reduced amount of cobalt carboxylate as compared to N,N′-[1,3-phenylenebis(methylene)] bis(isoindolin-1-one). The OS may be poly[(5-hydroxy-1,3-phenylene)bis(methylene) adipamide] (14) (i.e. MXD6 polyamide with the benzene ring derivatized with a hydroxy group). In an embodiment, 14 may be synthesized by subjecting MXD6 polyamide with a conventional benzene bromination reaction to form Br-substituted MXD6, which is then hydrolyzed into 14. The more hydroxy groups are present, the higher activity 14 will have, although higher amount of degradants may also occur. In an embodiment, the amount of hydroxy group may be adjusted by tuning parameters of the bromination reaction based on a tradeoff between these two consequences. Similarly, the OS may include tris(4-hydroxyphenylmethyl)amine (15), N,N,N′,N′-Tetra(4-hydroxyphenylmethyl)ethylenediamine (16), N,N′-dibenzyladipamide (17), or similar.

In some embodiments, the OS described above and those in the '209 application may be implemented as one of several oxygen scavenging active sites of a larger OS. This larger OS may be a molecule having several of those described OS linked together, an oligomer of the described OS, a polymer of the described OS or a dendrimer of the described OS. The disclosure below refers to “multiple C₁” synonymously with “multiple oxygen scavenging active site”. In other words, each C₁ represent one oxygen scavenging active sites. Each of the multiple C₁ atoms may be independently chosen from one of the described OS described above or described in the '209 application. Without being limited by theory, the multiple oxygen scavenging sites may work independently, and also, in some embodiments, work synergistically as described below. For example, one C₁ atom may be considered to be an activator for another C atom. In some embodiments, four C₁ atoms may be “activating” each other, similar to an activator activating a C₁ atom described with regards to formula (IV). For example, the multiple C₁ atoms may have the formula (IV-1), (IV-2) or (IV-3), where one of the C₁ atom is the C₂ atom described above.

In some embodiments, the active scavenging sites (C₁ atoms) are conjugatively attached to each other. For example, the multiple C₁ atoms are conjugatively coupled to each other. Accordingly, the multiple C₁ atoms may be in electronic communication, such as via electron delocalization, or via electronic resonance structures, with each other. For example, three C₁ atoms of three active scavenging sites are each directly linked to a same benzene ring, such that the three C₁ atoms are in electronic communication with each other (such as through resonance structure) through the benzene ring. In some embodiments, the three C₁ atoms may be ortho- and para- to each other. However, they may also be meta- to each other. In some embodiments, these active scavenging are each directly attached to a same nitrogen atom. In some embodiments, one active scavenging site is conjugatively attached to an aromatic ring, and one active scavenging site is conjugatively attached to a heteroatom, where the heteroatom is conjugatively attached to the aromatic ring. In some embodiments, these active scavenging sites are in proximity with each other. For example, these active scavenging sites (the two C₁ atoms) may be within two C—C bond distances from each other. In some embodiments, the two C₁ atoms may be directly connected to each other. For example, these active scavenging sites may be connected through several bonds, but the geometric configuration allows or enables these active scavenging sites to come close to each other such that they are within two C—C bond distances from each other in space.

In some embodiments, the multiple C₁ atoms may be part of an oligomer, a polymer or polymer fragment. For example, the polymer may include multiple side groups, each side group includes a C₁ atom of an oxygen scavenging moiety. Each of the oxygen scavenging moieties may be the same or different from each other. The C₁ atoms may not necessarily be conjugatively attached to each other. The density of the oxygen scavenging moieties are controlled to achieve the designed functionality. In some embodiments, the polymer may include multiple C atoms on the polymer backbone and the C₁ atoms are conjugatively attached to each other. In some embodiments, the multiple C₁ atoms are conjugatively coupled with each other.

In some embodiments, the OS includes multiple C₁ atoms, and each of the C₁ atoms are conjugatively coupled to a plurality of activator atoms. The multiple C₁ atoms may be part of non-polymeric molecule. For example, the OS may be N,N′-bis(3,4,5-trihydroxyphenylmethyl) pyromellitic diimide (or alternatively, 2,6-di(3,4,5-trihydroxyphenylmethyl)-pyrrolo[3,4-f]isoindole-1,3,5,7-tetraone (76). In some embodiments, the ester of this product may be first synthesized from a fine powder of mixed pyromellitic diimide (1 eq.) and anhydrous potassium carbonate (2 eq) with 3,4,5-Trimethoxybenzyl chloride (4 eq.) under gentle reflux for about 4 hours (using the first step of the Gabriel method for benzylamine). The ester is then hydrolyzed in high temperature water in a Parr bomb to provide the product. In some embodiments, the OS may be N,N′,N″,N′″-tetrakis(3,4,5-trihydroxyphenylmethyl)-1,2,4,5-benzenetetracarboxamide. In some embodiments, the OS may be 2,5-biscarboxy-N,N′-bis(3,4,5-trihydroxyphenylmethyl)-1,4-benzenetetracarboxamide. The carboxy group may be used to further derivatize the OS subsequently, such as to form a polymer, to tether additional oxygen scavenging functionality, and/or tether other functionalities. In some embodiments, the multiple C atoms may be attached to a phthalimide dangling oxygen. For example, the OS may be N,N′-bis(3,4,5-trihydroxyphenylmethoxy)pyromellitic diimide. In some embodiments, the synthesis may be substantially similar to that described above for N-benzyloxyphthalimide with derivatized starting material.

In some embodiments, the multiple C₁ atoms may be part of a polymer. For example, the OS may be a condensation polymer from trimellitic anhydride and xylylenediamines. Accordingly, the multiple C₁ atoms may be conjugatively attached (such as conjugatively coupled). In some embodiments, the polymer may have a flexible backbone, for example, having saturated C—C segments (e.g. saturated C₃-C₂₀ carbon chain segments) in its backbones. Accordingly, the multiple C₁ atoms may be positioned in proximity with each other. In some embodiments, the polymer structure is designed to include saturated C—C segments (e.g. saturated C₃-C₈ carbon chain segments). The shorter chain affords more predictability in the control of the structural orientations of bonds of the polymer, such that the multiple C₁ atoms are placed in proximity with each other. In some embodiments, the multiple C₁ atoms may be part of a dendrimer. For example, dendrimers are structural motifs that incorporate many same or similar functionality groups. The synthesis of dendrimers may use divergent or convergent approaches. The synthesis of dendrimer here starts from an OS precursor that includes a functional group that will be attached to the core or branch of the dendrimer. Dendrimer reactions often employ facile reactions (such as condensation reactions, click chemistry, coupling reaction, addition reaction, etc.) that are compatible with reactions described here. However, any suitable methods may be employed to synthesize the dendrimer. The article titled “Dendrimers: synthesis, applications, and properties” by Elham Abbasi, el. al. Nanoscale Res Lett. 2014; 9(1): 247 reviewed some aspects of the dendrimers and is incorporate herein in its entirety for reference. As compared simple polymer or oligomer structural motifs, dendrimers allow more precise control of the relative positions of the multiple C atoms such that they are positioned closer to each other. Without being limited by theory, having the multiple C₁ atoms in proximity improves the activity (such as the initiation and/or the reaction) of the oxygen scavenging reactions.

Likewise, in some embodiments, the multiple OS site may include C₃, and otherwise similarly configured.

In some embodiments, those OS may instead be incorporated onto or into a polymeric, oligomeric, or dendrimeric structure such that multiple OS active sites are present within one chemical structure. The scavenging molecule may be prepared using a precursor that includes the scavenging moiety and a connection portion. The connection portion may be configured to react with a functional group of a backbone or sidechain of a large molecule (such as polymer). Additionally, the connection portion may be configured to react with an end group of the large molecule. The end group may be a native end group of a monomer unit, or may be an end group formed during the application or degradation of the large molecule. Any suitable reaction that forms covalent bonds between monomers (such as condensation reaction, click reaction, coupling reaction, addition reaction, etc.) may be used. For example, the reaction may be between a hydroxy group and an anhydride group, an amine group and an ester group, an amine group and a double bond, a carboxyl group and a double bond, a hydroxy group and a double bond, a double bond and a diene (e.g. Diels Alder reaction), etc. Accordingly, an exchange reaction, an esterification reaction, a ring opening reaction, an addition reaction or other suitable reactions cause the scavenging moiety to be tethered. The connection portion may form part of the OS moiety, be connected to the OS moiety, or may be remote from the OS moiety.

In some embodiments, the scavenging molecule may include a scavenging moiety tethered to a backbone component either directly or through a linking or bridging group. For example, an OS moiety may be linked to an end group of a polymer, such as the hydroxy end group of a polyethylene glycol, double bond of the polyolefin, hydroxy end group of a polyester, double bond end group of a degraded polyester, etc. In some embodiments, the tethering may be accomplished by reacting a precursor to the oxygen scavenging moiety (such as an amine precursor to the OS) with the carrier resin (such as polyethylene terephthalate, polylactic acids, or polyacrylic acid, etc.) via aminolysis reactions. The amount of the amine precursor to the OS is controlled to balance for the activity, capacity, the polymer chain length, as well as the amount of degradants upon exhaustion of the oxygen scavenging capacity. In some embodiments, the amount of the amine precursor to the OS may be controlled based on a change of the intrinsic viscosity of the polymer, such as it does not drop below a certain threshold. In some embodiments, excess amount of amine precursor is used, and the polymer backbone (such as that of PET, PLA, etc.) may be degraded. Accordingly, small molecular OS are received, similar to those described elsewhere of the disclosure. In some embodiments, polylactic acid reaction with benzylamine forms N-benzyl-2-hydroxypropionamide as a small molecular OS. In some embodiments, instead of aminolysis reaction, addition reaction may be employed to tether the OS. For example, polyesters, polyolefins often include double bonds as the end group. These end groups may undergo addition reactions (such as hydroamination, Diels-Alder reaction, etc.). Particularly, polyolefins with tethered oxygen scavenging moieties may be used as OS for polyolefin applications (such as packaging material which has been a challenge of the industry). In some embodiments, recycled polymers (such as recycled PET, polyethylene, or polypropylene) may include large amounts of double bond end groups. They may be used to tether the oxygen scavenging moieties (e.g. forming polyethylene-CH₂—CH-benzylamine or polyethylene terephthalate-CH₂—CH-benzylamine by reaction of polyethylene or polyethylene terephthalate, respectively with benzylamine) and serve as a larger version (e.g. polymeric version) of the OS to be subsequently used in applications. In some embodiments, dibenzylamine is used instead to receive dibenzylamine as the oxygen scavenging moiety. This presents a unique opportunity to integrate the plastics recycling industry and the plastics additive industry.

In some embodiments, multiple scavenging molecules are tethered to a same backbone component, for example, at a nexus (or, core, nucleus, etc.). In some embodiments, the scavenging molecules may be immediately and directly attached to the nexus, while in other embodiments, the scavenging moiety may be remote to the nexus (such as through linking or bridging groups). In some embodiments, there may be multiple nexuses. In some embodiments, there may be no clear nexus. For example, multiple oxygen scavenging moieties are linked to multiple sites on a polymer backbone. For example, the backbone may be a polyvinylalcohol, and the multiple oxygen scavenging moieties may be linked to the hydroxy oxygen of the polyvinylalcohol. In some embodiments, the nexus is a cyanuric acid with three scavenging moieties attached to the hydroxy oxygen. In some embodiments, the nexus is an isocyanuric acid with three scavenging moieties attached to the amide nitrogen. In some embodiment, the nexus is a triglycerol with the oxygen scavenging moiety attached to the hydroxy oxygen atoms. In some embodiments, the nexus is a pyromellitic anhydride (PMDA) core or a tetracarboxylic benzene core (product from PMDA).

In some embodiments, the OS includes isoindoline- or phthalimidine-attached siloxane, disiloxane, oligosiloxane, or polysiloxane. For example, phthalimide may react with dimethyldichlorosilane, and the product may be reduced with hydrogen in presence of nickel-based catalyst to form the OS including C₁ active sites and siloxane component(s). In some embodiments, phthalic anhydride may react with allylamine. The product is then reduced using PMHS with less than 0.1% Karstedt's catalyst. Accordingly, the OS is tethered to the PMHS backbone. In some embodiments, the OS is a copolymer having a polyalkyleneamino segment directly attached to a carbonyl, thereby forming amides. For example, the copolymer includes —C₆H₄—(C═O)—N—(CH₂)ₙ—N—(C═O)—C₆H₄— segment. For another example, the copolymer includes13 C₆H₄—(C═O)—N—(CH₂)ₙ—N—(C═N)—C₆H₄— segment. In some embodiments, n is about 1 to 3. In some embodiments, n is about 4-5. In some embodiments, n is about 5 to 20. Short chains may provide better synergistics in some applications; conversely, longer chain may be beneficial depending on the base polymer structure due to its flexibility. In some embodiments, 1,4-diaminobutane is condensed with terephthalic acid to form the OS. In some embodiments, 1,4-diaminobutane is reacted with PET (e.g. recycled PET) to form the OS.

As described above, the oxygen scavenging moiety may be formed on periphery of the dendrimers. In some embodiments, the oxygen scavenging moiety may alternatively or additionally be formed on inner portions of the dendrimers. In some embodiments, the terminal or surface portion of the dendrimer may have reduced or no oxygen scavenging moieties. In some embodiments, degradants may be formed during the oxygen scavenging reaction. Having the scavenging moieties embedded within inner structure of the dendrimer may trap the degradants and mitigate their migration out of the plastics and into the contents of the packaging (or otherwise other contents utilizing the functionality of the OS). This may be important to certain applications, like pharmaceutical applications, food and beverage applications, etc. In some embodiments, this may be achieved by first forming a nexus having an oxygen scavenging moiety or multiple oxygen scavenging moieties, along with activators if appropriate, and then grow dendrimers therefrom. In some embodiments, a dendrimer inner sphere is first formed. Then, a layer of OS is attached to the inner sphere. The OS may be ditopic (or having at least two functional groups—one used to attach to the inner sphere, and the other(s) used to further grow the dendrimer). In some embodiments, a radical reaction catalyst (such as those described later) is incorporated in the dendrimer structure. For example, a transition metal may be formed in proximity of the OS moiety on the dendrimer. For the situation where the OS is located on an inner sphere of the dendrimer, the catalyst may be incorporated close to the OS layer. In some embodiments, the dendrimer is configured to include sites that may be used to attach a transition metal. For example, a carboxylic group may be incorporated close to where the oxygen scavenging moieties are. For example, the nexus of the dendrimer may include a free carboxylic acid group (e.g. when the dendrimer formation does not use up that carboxylic acid group either due to dendrimer does not grow on such motif, or by controlling the stoichiometries of reaction). Then the dendrimer nexus is exposed to transition metal salt (such as cobalt acetate) such that the transition metal salt is tethered by ionic interaction onto the dendrimer core. Then the oxygen scavenging layer is grown over and trapping in the transition metal catalyst. Further, additional dendrimer layers are grown over the oxygen scavenging layer. In some embodiments, the ratio of the oxygen scavenging moiety and the catalyst is optimized, for example, using the ladder method of the '209 application. In some embodiments, the ratio between the number of C₁—H bonds in the dendrimer to the catalyst may be about 5000:1 to about 100:1 for room temperature food and beverage packaging applications in PET plastics. In some embodiments, the ratio may be about 1000:1 to about 300:1. These threshold values may be modified for different applications and/or different application conditions. In some embodiments, another layer or layers of inert dendrimer material is grown that wraps in the oxygen scavenging layer. In some embodiments, the scavenging moieties do not require an oxidation catalyst to initiate and complete the scavenging reactions. For example, the scavenging moieties may be those described in the 209' application having a small BDE, such as imines.

The scavenging moieties may be any suitable OS moieties having suitable motif that connects to, directly or indirectly, to the backbone. In some embodiments, there is a linking group (or atom) distinct from the oxygen scavenging moiety that connects the oxygen scavenging moiety onto the backbone (polymer, dendrimer, etc.). In other words, the linking group (or atom) does not contribute to the oxygen scavenging activity. In some embodiments, the linking group (e.g. the hydroxy oxygen, the amide nitrogen, etc.) may form part of the functional scavenging moiety, and contribute at least partially to the activity. In such embodiments, the oxygen scavenging moiety may be constructed during the tethering reaction to the backbones. The scavenging moieties may be those described herein or may be those functional oxygen scavenging molecules previously described. In some embodiments, the scavenging moiety (either wholly from the precursor or constructed during the tethering reaction) includes at least one ring. Accordingly, in some embodiments, degradants from the oxygen scavenging reactions may not freely migrate away from the backbone. This may be particularly useful for certain applications, such as pharmaceutical and/or food and beverage applications. In some embodiments, the scavenging moiety is tethered onto a polyester, polyamide, polyurethane, polyacrylate, etc. Such OS may be particularly useful for applications using polyester, polyamide, polyurethane, polyacrylate, respectively, as the main matrix. In some other embodiments, the scavenging moiety is tethered onto a polyolefin. These materials may be applied as oxygen scavenging compositions for polyolefins to improve polymer compatibility. As described above, any tethering methods may be used to tether onto the backbone, the side chain, or end groups (such as double bond end groups) of the polymer.

In some embodiments, the tethered OS are formed as a standalone scavenger to be applied as an oxygen absorber, such as a sachet or insert. In some embodiments, the tethered OS are formed as part of a composition. In some embodiments, these tethered OS may be processed into a masterbatch (such as a polymer masterbatch), either themselves alone, or with additional carrier plastics, for easy introduction into the processing. In some embodiments, rather than tethering to include the oxygen scavenging moieties on the polymer, oligomer, or dendrimer motifs, oxygen scavenging moieties may instead be dissolved or suspended in these larger molecules. For example, polyethylene glycol, waxes, etc. may be used to carry the OS to allow them to be incorporated into the main plastics.

The OS may be used as a standalone oxygen absorber. In some embodiments, they may be used in PET packaging. In some embodiments, the oxygen scavenging composition may be applied, instead of in PET, in other base resins. For example, they may be applied in polyolefin (such as high density polyethylene (HDPE), low density polyethylene (LDPE), polypropylene, polystyrene, etc.) resins. In some embodiments, as described above, the OS are tethered onto an oligomer (or short-chain polyolefin) of the polyolefin. The oligomer is then used as an additive to be dosed into the main polyolefin resin in processing. These polyolefins may be used for, for example, fuel containers, fuel additive containers, milk bottles, etc. In some embodiments, these applications may implement monolayer structures for the ease of fabrication. In some other embodiments, these applications may implement multilayer structures to improve barrier properties.

In some embodiments, such base resin may be used to cover an article, such as a PET article. For example, shrink wraps are often used for advertisement purposes. Shrink wraps may include the oxygen scavenging composition so as to provide oxygen barrier property to the otherwise non-barrier PET articles. In some embodiments, the shrink wraps may implement an NPG polyester, a PMMA base resin, etc. as the base resin. The identity of the base resin, the processing parameters, the oxygen scavenging dosage may be determined based on the glass-transition temperature of the base resin, the shrinkability, the tensile stress of the base resin, the melt temperature, the expected shelf life, among others. In some embodiments, the base resin itself is modified to form an oxygen scavenging moiety. For example, the polyacrylate base resin may react with benzylamine to form OS-tethered base resins for shrink wraps. For another example, the polyacrylate base resin may react with allylamine to form OS-tethered base resins for shrink wraps. For example, any other oxygen scavenging moieties may be derivatized and incorporated.

In some embodiments, the OS may be designed to provide color change as a result of its reaction(s) with the oxygen. For example, the OS undergoes an oxidative additional reaction, such as an oxidative intramolecular cyclization reaction. Accordingly, the color change may indicate the consumption of the OS, and thereby indicating the remaining capacity of the OS. This further indicates the remaining shelf life of, for example, the content of the packaging. Without being limited by theory, the oxygen scavenging reaction causes the structure (V) to undergo an internal cyclization reaction, thereby increase the size of the conjugation system. As a result, the color changes. For example, the OS may have the following formula (V)

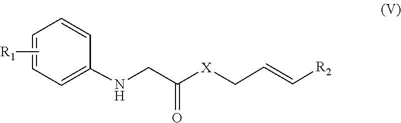

In some embodiments, X is oxygen, and the R₁ is hydrogen. In some embodiments, the R₁ is halogen. In some embodiments, the R₁ is 4-methyl, 4-methoxy, or 2-methoxy. In some embodiments, the OS includes an activator (4-hydroxy). In some embodiments, the R₂ is a phenyl group.

In some embodiments, R₂ is phenyl. The reaction starts with forming cinnamyl 2-bromoacetate. For example: In some embodiments, cinnamyl alcohol (26.85 g, 200.0 mmol) and pyridine (15.83 g, 200.0 mmol) are dissolved in anhydrous methylene chloride (150 mL). 2-bromoacetyl bromide (6.78 g, 60.0 mmol) is slowly added into the solution at 0° C. under nitrogen atmosphere over 50 min. The reaction mixture is then stirred at room temperature for about 6 hours, and the reaction progress is monitored by TLC. Upon completion, the reaction mixture is washed with water, dried over sodium sulfate. The solvent is removed under reduced pressure to afford cinnamyl 2-bromoacetate. The cinnamyl 2-bromoacetate is then reacted with any suitable substituted anilines to generate the OS. In one embodiment, the OS has 4-methoxy as the R₁ group. The cinnamyl 2-bromoacetate is reacted with p-anisidine (1 eq.) and anhydrous potassium carbonate (1.5 eq.) in dry acetone under refluxing condition for 20 h. The reaction mixture is filtered, and the filtrate is concentrated and then poured onto ice-cold water with rigorous stirring. The organic layer is then extracted with diethyl ether. The ether layer is washed with 5% hydrochloric acid, dried over anhydrous sodium sulfate, and evaporated to form the product. In some embodiments, the OS has methyl as the R₁ and phenyl as the R₂. The cinnamyl 2-bromoacetate (14.04 g, 55 mmol) is mixed with potassium carbonate (8.30 g, 60 mmol), potassium iodide (9.13 g, 55 mmol), p-toluidine (5.36 g, 50 mmol) in acetone (150 mL). The mixture is heated to reflux and maintained at refluxing condition for about 8 hours under inert atmosphere. Upon completion as indicated TLC monitoring, the mixture is cooled to room temperature, filtered, and washed with acetone. The solvent is then removed under reduced pressure, and the crude product worked up. The product is cinnamyl 2-(p-tolylamino)acetate as a white solid. In some embodiments, the R₁ is 4-tert-butyl and R₂ is phenyl, and the OS is synthesized using similar methods with the exception of the p-toluidine is replaced with 4-tert-butylaniline. Similarly, when R₂ is phenyl, the R₁ may be 4-Phenyl (which is a yellow solid), 2,4-Dimethyl (which is a red oil), 4-Chloro (which is a white solid), 4-Bromo (which is a white solid); 2-bromo-4-methyl (which is a red oil), 4-bromo-2-methyl (which is a red oil), each synthesized using the same procedure with the corresponding substituted aniline.

In some embodiments, the R₁ is 4-methyl, and the R₂ varies with several options. The method described above is followed, although derivatized cinnamyl alcohol replaces cinnamyl alcohol above, and p-toluidine replaces p-anisidine. For example, while R₁ is 4-methyl, R₂ may be a 4-Fluorophenyl (which is a yellow solid), 4-Chlorophenyl (which is a yellow solid), 4-Bromophenyl (which is a light yellow solid), 4-Methoxyphenyl (which is a white solid), 2-Methoxyphenyl which is a red oil), Thiophene-2-yl (which is a yellow solid), 1-propene-1-yl (which is a red oil), or 2-phenylethene-1-yl (which is a yellow solid), each synthesized using the same procedure with the exception of the starting material bearing the R₂. In some embodiments, the oxygen scavenger is an amide analog of the above molecule. In other words, X may be —NR₃—. As described above, these products change color as their oxygen scavenging capacity degrades, thereby indicating a remaining shelf life of the material they protect. In some embodiments, rather than undergoing an intramolecular oxidative addition reaction, the oxygen scavenging moiety under goes oxidative addition (such as oxidative cyclization) reaction with a scaffold it attaches to (such as the dendrimer, polymer, or carrier). In some embodiments, the oxygen scavenger undergoes an intermolecular addition reaction (such as an oxidative addition reaction with the polymer backbone.

As described later, OS may also serve as radical scavengers (hereinafter referred to as “RS”). RS are often referred to as antioxidants, thermal stabilizers, light stabilizers (e.g. sterically hindered amines or phenols, phosphite esters, organosulfurs), UV absorbers (e.g. benzotriazoles and benzophenones), metal scavengers, and other. They may be used to protect the resins from thermal or light initiated degradations. RS and OS are very different. For example, unlike most OS that require the presence of a radical catalyst (except some molecules described here or in PCT/US20/15209 due to drastically higher activities), RS generally requires the absence of the radical catalyst. Typical RS targets to inhibit radical reactions such that the presence of a radical catalyst often adversely affect its activity. Moreover, unlike OS applications that typically involve ambient or near ambient conditions, most RS application involve substantial energy inputs, such as at the melt processing temperature of the resins, or under direct sunlight with substantial amount of photon energy. With these clear distinctions, no surprise that all known commercial RS to the inventor are inactive and generally inhibitive to OS applications. Therefore, literature that describes radical scavengers for high-temperature applications likely do not provide a functional OS. Moreover, literature compositions that include metal scavengers or metal deactivators likely do not provide a functional OS. However, the inventor surprisingly discovered that all evaluated OS (whether disclosed here or elsewhere) do function as RS, when used in absence of radical-based oxidation catalysts. Such use of OS in RS applications is not known to the industry. For example, no commercial OS has been used as RS as thermal stabilizers, with or without, the presence of any oxidation catalyst to the best knowledge of the inventor. Based on the above, all OS described here throughout the disclosure or those “oxidizable additive” described in PCT/US20/15209 function as OS in presence of an oxidation catalyst, and also as RS in absence of the radical-based oxidation catalyst. This unique dual functionality lies in the presence of C₁ or C₃ active site which are often missing in commercial RS; by contrast those commercial RS are often radical deactivators that interfere with OS reactions. Despite this dual functionality, for simplicity only, the disclosure has been and will be referring to a material solely as OS or RS based on the relevant application in context. Such labels shall not be construed to limit the material to either application scenario. Rather, unless explicitly indicated otherwise, the description of either “OS” or “RS” shall be construed to provide, simultaneously, a useful material for OS applications (e.g. shelf-life extension) (where the presence of an oxidation catalyst is assumed or unnecessary), and a useful material for RS application (e.g. thermal stabilizer, antioxidant, UV block) (where the absence of OS catalyst is assumed). Accordingly, a description of an OS in one part of the disclosure shall be combinable with another embodiment of the disclosure that uses the RS terminology. Moreover, any product or processing format (e.g. discrete molecule, polymer, dendrimer, tethering, additive, coextrusion described above equally applies to RS.

With respect to OS applications, the formulation may include conventionally known OS catalysts, provided that their dosage is adjusted based on the target application. For example, the catalyst may include salts (e.g. carboxylate salts) or complexes of Cobalt (Co), Iron (Fe), Manganese (Mn), Titanium (Ti(III)), Vanadium (V), Copper (Cu(I)), other suitable radical reaction catalysts, or combinations thereof. As described, the radical reaction catalysts may be omitted, for example, when the OS has particularly high reactivities (such as having a BDE less than a threshold determined based on the application, e.g. imine-based (e.g. benzyl benzylidine for room temperature applications)) and/or the application temperature is relatively high (such as the “hot tea” “hot coffee” “hot chocolate” applications). In some embodiments, the catalyst is not associated with the OS. For example, they may be introduced into the processing of a packaging article using a masterbatch containing the oxidation catalyst. The masterbatch does not include the OS. However, in some embodiments, the OS and the catalysts are associated with each other. Having the catalyst associated with the OS improves the probability of initiation and/or completion of the scavenging reaction. For example, as described above, the OS may include a dendrimer. The dendrimer may incorporate the catalyst within its pores. For another example, the OS may be tethered onto a cage molecule or a supramolecule which incorporates the catalyst within its structure. In some embodiments, the catalyst is incorporated with polymeric OS by derivatizing the backbone to include, for example, carboxylic acid groups, or sulfonate groups, which then interact with the catalyst and be so associated. As described earlier, the amount of the oxidation catalyst may be adjusted to optimize the performance of the OS. In some embodiments, any of the formulations above may include additional additives, such as colorants, anti-slip additives, reheat additives, stabilizers, etc. With respect to RS applications, radical scavengers may be used, such as phosphorus (e.g. triphenyl phosphate) or similar. In some embodiments, the incorporation of activator groups described above may further improve efficacy.

The RS applications include reducing plastics wastes from degradations. PET is one of the most recycled plastics, yet still less than 30% of PET is recycled worldwide, of which the majority is recycled mechanically. The mechanically recycled PET often suffers reduced strengths, degraded rheology, and increased darkness even after proper treatments by current standards. After several cycles of mechanical recycling, the material may be too much degraded to be mechanically recycled again. Most of the time, these materials can only be landfilled. Therefore, mitigating degradation during the use and mechanical recycling increase the useful life of the PET and reduce the environmental impacts. The degradation of PET largely originates from chain scission and the active end groups produced during the chain scissions. In an aspect, the disclosure provides methods of mitigating such chain scissions. It is noted that radical-based degradation is not unique to PET or polyesters. Most polymers undergo similar degradations in thermal processing, under light exposure, or in normal usages. Accordingly, the above aspect of the invention may be suitable for other polymers as well.

In an embodiment, an RS component is included in the PET matrix in absence of an OS catalyst. The RS may interact with the radicals produced during the processing, normal application/use, or during the recycling of PET, to either consume or deactivate them, thereby protecting the backbones of the PET chains from radical degradation. Accordingly, the PET is stabilized and can potentially be mechanically recycled for more cycles than without implementing the methods. In an embodiment, the RS may be one of those described above, or may be one of those described in PCT/US20/15209. In some embodiments, the RS may be one of those commonly known as OS or organic oxygen absorbers elsewhere but without presence of the OS catalyst. This includes, for example, MXD6, Amosorb®, OxyClear®, DiamondClear®, MonOxbar®, Oxbar®, etc.

In some embodiments, the RS is introduced to the PET during the recycling operation. In some embodiments, such introduction includes using an exchange catalyst to facilitate reaction between end groups of PET and an end group of the RS. In an embodiment, the PET structure may further be modified to reduce its degradation. As described above, the manufacturing process of virgin PET may be modified to improve downstream recycling (e.g. by incorporating RS). In an embodiment, endgroups of PET may be capped toward that goal. For example, conventional PET may have end groups of carboxy (—COOH) and/or hydroxy (—OH). Their presence provides the opportunity to increase molecular weight during the manufacturing process. However, they also largely contribute to the PET degradation during mechanical recycling. In a first aspect, PET is endcapped at the end of its manufacturing, e.g. at the end of the solid state polymerization (SSP), or where SSP is not implemented, the end of the polycondensation. Alternatively, PET may be endcapped after the manufacturing process has completed in a separate processing. The —OH may be endcapped with triflic acid, naphthalic acid, benzoic acid, stearic acid, other monocarboxylic acid, or similar groups; the —COOH may be endcapped with carbodiimide, monoalcohol (or alkoxide), N-substituted lactam, oxazoline, imidazole, or the like. Moreover, the —COOH may be endcapped with oxirane or cyclic alkylene carbonates, which turns the —COOH end group into —OH group and may be further endcapped as the —OH group. The endcapping remove the concentration of endgroups thereby reducing endgroup-induced degradations.

In some embodiments, it may be further desirable to remove the capping group subsequently. For example, removing the end capping allows the —COOH and/or —OH groups to be regenerated which may be necessary to increase the chain length of the polymer (e.g. in recycling applications to upgrade the polymer) or to prepare copolymers by introducing additional comonomer or functional groups, e.g. OS or RS to interact with the regenerated endgroups. Accordingly, it may be beneficial that the capping groups are easily removed, ideally at recycling conditions. In one embodiment, the PET may be synthesized to include mostly —OH end groups. Accordingly, at the end of the condensation reaction, the majority of the end groups are —OH end groups. The —OH end groups are capped using silyl groups (such as Trimethylsilyl (TMS), Triethylsilyl (TES),Triisopropylsilyl (TIPS), Dimethylisopropylsilyl (IPDMS), Diethylisopropylsilyl (DEIP 5), t-Butyldimethylsilyl (TBS), t-Butyldiphenylsilyl (TBDPS), Tetraisopropyldisiloxanylidene (TIPDS), Di-t-butylsilylene (DTBS), or similar. In an embodiment, the silyl groups may be introduced following Corey, E. J.; Venkateswarlu, A. J. Am. Chem. Soc. 1972, 94, 6190 or Corey, E. J.; Cho, H.; Rücker, C.; Hua, D. H. Tetrahedron Lett. 1981, 22, 3455. In one alternative example, after the completion of the polycondensation reaction, silyl chloride is introduced into the molten polymer. The reaction is maintained for a period of time under agitation, and an extra amount of silyl chloride, as well as reaction byproducts are removed by vacuum. In some embodiments, it may be beneficial to avoid the use of silyl chloride or similar typical silylation reagents (e.g. to avoid the evolution of HCl). Mono-silyl-derivatized ethylene glycol may instead be used in its place, which may be prepared following, e.g. McDougal, P. G.; Rico, J. G.; Oh, Y.; Condon, B. D. J. Org. Chem. 1986, 51, 3388 or Hu, L.; Liu, B.; Yu, C. Tetrahedron Lett. 2000, 41, 4281. Because of the free hydroxy group, mono-silyl-derivatized ethylene glycol may react substantially similarly as ethylene glycol in the capping process. In some embodiments, the process described here are performed during the final stage of the preparation. For example, where SSP is not implemented, this process may occur at the end of the polycondensation phase; where the SSP is implemented, the process may be implemented at the end of the SSP. In some embodiments, this process may occur at a temperature above the melting point of PET or below (e.g. between Tg and Tm). The higher temperature facilitates the reaction yet in some instances causes degradation issues; the lower temperature may lead to slow reaction and incomplete derivatization. For example, the derivatization may occur at about 100° C. to about 280° C. The resultant material undergoes the same processing and produces PET pellets, fibers, films, sheets, or any other suitable format. In one embodiment, the PET may be synthesized to include mostly —COOH end groups. The —COOH may be similarly capped using the silyl groups by introducing the silyl chloride. Still alternatively, the stoichiometric of the PET raw materials are configured such that the PET include —COOH and —OH end groups at about 1:1 ratio. Similarly, both types of end groups may be capped using silyl chloride as the capping agent. Because of the lack of active end groups, the degradation of the polymer is reduced. In some embodiments, the introduction of the silyl groups may be at the end of the mechanical recycling operation. The end capping may be introduced in a batch reactor or as in an extrusion process. Alternatively, instead of capping with an easily cleavable capping group like a silyl, the end groups may be capped with a component that converts into a new end group capable of further condensation. In some embodiments, the end capping group may also be an OS or RS. For example, as described above, 21 may be introduced as such an end cap group to react with —COOH end groups. In some embodiments, an exchange catalyst is introduced along with 21 to PET to facilitate the capping reaction. During processing or the application, 21 is gradually converted into a new end group (phthalic anhydride) by oxidation, thus enabling the further condensation of the otherwise already-capped the PET with either newly generated HO— end groups on the PET, or with diols introduced at a subsequent processing stage.

In some embodiments, the PET prepared this way (and molded articles therefrom) undergoes degradation to a lesser extent as compared to other approaches. Nevertheless, overtime, the PET still undergoes degradation, such as due to thermal-related or light-induced cleavages to generate new end groups, and/or other oxidative and/or reductive modifications. In a recycling operation, it may be desirable to maximally condense so as to increase in molecular weight and restore the material strength and intrinsic viscosity. For this purpose, it may be desirable to remove the capping groups such that more end groups are available to condense with one another. In other words, it may be desirable to remove the capping groups. The silyl groups described here may be removed (or cleaved) when necessary using basic (e.g. 5% NaOH-95% MeOH) or acidic (1% HCl-MeOH, 25° C.) conditions. Coincidentally, the conventional mechanical recycling operation typically implements an alkaline wash or an acid wash of the used PET, which completes the cleavage of the protecting group. In other words, without the need of modifying the mechanical recycling process, by simply implementing the silyl capping group at the manufacturing stage, PET degradation during processing and usage may be inhibited to maximally preserve molecular structure; and when it is later recycled, the capping groups are removed, such that no interference with the upgrade or condensation will occur. The PET value chain is therefore improved with minimal cost. Accordingly, as compared to other capping technologies, silyl-protective group provides additional benefit. Moreover, generally, silyl-protected —COOH may be more easily cleaved than silyl-protected —OH groups. Accordingly, configuring PET to have mostly —COOH endgroups and form silyl-capped —COOH may be further beneficial in certain circumstances. It is noted that in the above-described process, only the capping groups necessary to be cleaved for the subsequent condensation is removed. For example, such subsequent condensation or upgrade process mostly occur at the interface between washed PET flakes (or pieces of other shapes). Accordingly, there is no need to cleave the silyl capping group deep inside the flakes. Here, the acid wash or alkaline wash indeed only cleaves those on the surface of the PET flakes that are in contact with the washing solution. However, in some embodiments, the acid wash or alkaline wash may be modified to allow for more extensive cleavage, for example, when contamination is extensive. In some embodiments, because silyl ester groups (e.g. capped —COOH) have different lability from the silyl ether groups (e.g. capped —OH), this washing process may be configured to selectively remove only the silyl capping on the —COOH group or remove only the silyl cap on the —OH groups, or both. As described above, removing both caps allow for subsequent condensation and upgrade of the polymer by itself In some embodiments, removing only the silyl capping of the —COOH group allows dialcohol, diamine, or other ditopic materials capable of reacting with the —COOH groups to be introduced to upgrade the polymer as well as to impart new functional groups. For example, the OS components (e.g. 11, 12, or similar) may be incorporated in this process. It is noted that this aspect of the disclosure may be less suitable to polymers that do not have large numbers of active endgroups, such as additional polymers.

In some embodiments, the degradation of the PET overtime generates radicals which eventually generate vinyl end groups (e.g. benzoate vinyl end groups). Such vinyl end groups may ultimately lead to color in the recycled PET in conventional approaches. In some embodiments, the recycling of the PET includes a step at which the PET is reacted with itaconic acid, itaconic anhydride, or esters thereof. For example, the PET is subject to a reactive extrusion process in which a suitable amount of itaconic acid, anhydride, or ester is added. The condition of the reactive extrusion is configured to allow for radical reactions between the vinyl end groups and the double bond of the itaconic acid. In some embodiments, the itaconic acid, ester, or anhydride is configured based on the anticipated amount of the vinyl end groups (which may be correlated with the particular use of the PET prior to the recycling). For example, the amount of the itaconic acid is configured to be at an excess with respect to the amount of the vinyl end groups. In some embodiments, the amount of the itaconic acid may be at large excess with respect to the amount of vinyl end group. Itaconic acid as a comonomer has been recognized by FDA to be safe (see FCN 2034). In some embodiments, the reactive extrusion condition is further configured to encourage an exchange reaction between carboxylic acid group of the itaconic acid and the PET backbone. In some embodiments, the itaconic acid may be introduced into the extrusion and allowed to react with the vinyl end groups. Subsequently, an exchange catalyst is introduced. In some embodiments, instead of reactive extrusion, the above described process is performed in other suitable environment, such as in a batch reactor with similar materials, amounts, and sequences. In some embodiments, itaconic anhydride may be preferred due to its tendency to be incorporated into the polymer backbone with the presence of the anhydride group. For example, in some embodiments, the above process may be achieved without the use of an exchange catalyst with itaconic anhydride. In some embodiments, itaconic acid may be preferable due to its ditopic nature and economic considerations. In some embodiments, itaconic ester may be preferred due to its lack of reactive group. In some embodiments, the PET being recycled are primarily with HO— end groups. Accordingly, the HO— end group may react with the itaconic acid, anhydride, or ester. Alternatively, the PET may be —COOH end group, and the condensation reaction is inhibited in favor of the vinyl-removal process. In such alternative embodiments, it may be beneficial to not include an exchange catalyst. In some embodiments, the end groups have been silyl capped and have been regenerated during the process immediately prior to the process discussed in this paragraph. The itaconic moiety has C₁ active site. Accordingly, in some embodiments, unreacted itaconic acid in the PET structure serves as a RS or antioxidant during the thermal processing, and to some extent during the subsequent usage. In some embodiments, alternative or additional to itaconic acid, anhydride, and esters, an alkene (e.g. styrene) may be used to react with the vinyl end groups. In the case of styrene, the resulting product may be a PET-polystyrene copolymer, where styrene condenses with the vinyl end groups to form segments of the polystyrene. For example, the structure so formed may include a segment of styrene dimer, trimer, tetramer, or other oligomers (depending on the relative concentration of styrene) where one of the styrene phenyl group is replaced with benzoate group (e.g. the benzoate group being a terminal group of a PET chain). Therefore, the PET chain is connected to the segment. In some embodiments, the benzoate group may be cleaved off thereby forming a double bond with an adjacent carbon, for example, generating a —CH(-Ph)-CH═CH—CH₂— segment or a —CH(-Ph)-CH₂—CH═CH— segment, such as a —CH(-Ph)-CH═CH—CH₂—CH(-Ph)-CH₂— segment. As compared to other approaches not implementing embodiments described herein, the present approach has the benefit of generating less color body. In some embodiments, in addition to styrene (or a similar molecule), one or more molecules having the “activator” may be introduced. The activator facilitates the interaction between the vinyl end group and the styrene. However, in other embodiments, such an activator is not necessary. In some embodiments, the conditions of the reaction may be controlled to form only a short polystyrene segment, such that properties of PET are not substantially changed. This may be controlled by adjusting the amount of styrene monomer provided, reaction temperature, duration, and the amount or identity of the activator molecule (if used). In some embodiments, alkylene oxide (e.g. ethylene oxide), alkylene carbonate (e.g. ethylene carbonate), other materials capable of addition polymerization or ring-opening polymerization may be used in place of styrene. In some embodiments, these materials are used in conjunction with itaconic acid, anhydride, or ester. As compared to other methods of removing radicals (e.g. with phosphorous), the recycled resin may retain compatibility with subsequently introduced OS due to the absence of strong radical deactivators.

In an embodiment, the PET may alternatively or additionally be recycled chemically into the feedstock materials for virgin PET. This method may be used independent from mechanical recycling. Alternatively, this method may be used in conjunction with and following mechanical recycling e.g. repeated mechanical recycling operations. For example, using mechanical recycling (either conventional or those described above), PET may be recycled mechanically for several times. Although using the methods described above may enable the mechanical recycling to be conducted for several more times, eventually, the degradation may be such that further mechanical recycling is no longer feasible. Accordingly, chemical recycling may further be implemented to convert the otherwise non-recyclable materials into feedstock materials for virgin PET. The following may be performed on PET streams that are with or without the capping groups and with or without the OS or RS components.

Chemical recycling of PET into feedstock materials are well-known. However, the process typically involves energy-intensive processes for many hours to achieve not necessarily great yields. PET is merely an example. Here, improved methods are provided for condensation polymer, such as a polyester, using a mechanochemical method. In an embodiment, mechanochemical reactions are induced between PET and a hydroxide source material. Any suitable mechanochemical methods may be used such as grinding, milling, slicing, extruding, etc. The mechanochemical reactions may be carried out in planetary ball mills, centrifugal ball mill, attrition mills, tube mill, stirred media mills, rotary mills, other mills, pulveriser, grinder, extruder, or other suitable instruments. The process of extrusion may implement single screw extruder or twin screw extruders (with co-rotating or counter-rotating screws). The extrusion may implement reactive extrusion principles. In some embodiments, milling may be preferable in that reaction time may be substantially longer than that in the extrusion process such that complete reaction may be easier to be achieve; on the other hand, while extruder provides limited residence time, it is continuous and able to accurately control temperatures, therefore more amenable for large-scale processing of materials. Moreover, the different reaction conditions between milling and extrusion have been reported to lead to different reactivities and products from identical reactants.

In some embodiments, PET is mechanochemically reacted with an inorganic material in an extruder. In an embodiment, the reaction is conducted in an inert atmosphere without the presence of oxygen. In some embodiments, the inorganic material is an inorganic hydroxide, such as an alkali hydroxide. In some embodiments, the inorganic material is an alkaline earth metal hydroxide. The hydroxide attacks the PET to produce terephthalic salts and ethylene glycol. While inorganic hydroxide provides good reactivity towards PET depolymerization, their hazardous profile may be a safety and cost issue. In some embodiments, the inorganic material is a transition metal hydroxide with reduced corrosiveness. In some embodiments, the inorganic material may be a precursor to the hydroxide and selected based on its decomposition characteristics (e.g. decomposition temperature), decomposition product, melting temperature, crystallization structure (e.g. presence of crystallization water), the pKa of the respective conjugated acid, etc. In general, it is desirable to form hydroxide ions at the reactive condition either via thermal decompositions, or by equilibrium with the presence of water molecule. In an embodiment, the inorganic material is sodium bicarbonate. For example, an excess amount (e.g. about 1.5 eq. to about 3 eq. of NaHCO₃ and 1 eq. of PET) is extruded with a temperature profile that includes at least a portion above 80° C. at the entry point of the NaHCO₃. In an embodiment, NaHCO₃ decomposes at this temperature and the degradants include hydroxide ions that have improved reactivity towards PET. In an embodiment, the temperature in the zone where NaHCO₃ is injected is kept at less than 100° C. This reduces the evaporation of water produced that assists in the reaction with PET. In some embodiments, the degradants also include carbonate ions reacting with the PET. Moreover, the carbon dioxide generated also creates cavities that improve the mechanochemical reaction. In an embodiment, the entire screw length is at a temperature above Tg of PET. PET at a temperature higher than its glass transition temperature improves the material mixing. In an embodiment, the excess NaHCO₃ serves as a grinding assistant to improve the mechanochemical reaction. However, in some embodiments, the reaction may instead be stoichiometric at 1:1 molar ratio. In an embodiment, the inorganic bicarbonate is potassium bicarbonate (KHCO₃), and the temperature profile includes at least a portion above about 100° C. to about 120° C. close to the entry point of the KHCO₃. In an embodiment, PET is mechanically reacted with an inorganic carbonate (e.g. Na₂CO₃, K₂CO₃). In an embodiment, the inorganic carbonate may include at least 1 equivalence of crystallization water in its crystal structure. In an embodiment, up to 10 equivalences of crystallization water may be present. In an embodiment, anhydrous inorganic carbonate is used, although about 1˜10 eq. of water (relative to the carbonate) is injected along with the carbonate or separately from the entry point of inorganic carbonate. In an embodiment, the inorganic carbonate (with or without crystallization water) may be at an excess, where the excess amount serves as the grinding agent. The introduction of water improves the reactivity for the mechanochemical reaction by assisting the generation of hydroxide ions. In an embodiment, the extrusion may implement other bicarbonate or carbonate salts selected using the criteria described herein. At the exit of the extruder, terephthalate salts of the inorganic ions are received, which is mixed with ethylene glycol and inorganic salts that are easily separated using conventional methods and recovered. The terephthalate salts may be treated with acids to recover terephthalic acid (TA). In an embodiment, sodium formate may be used in place of the inorganic bicarbonate or carbonate. In an embodiment, similar parameters as those described above may be used for the mechanochemical reaction with sodium formates. In an embodiment, either hydrated sodium formate is used, or water (typically a few weight percent, e.g. 1% to 5%) is injected together. By using sodium formate, the end product may be TA (instead of salts thereof) without further work-up. In an embodiment, a smaller ratio between sodium formate to PET may be used. For example, a sub-stoichiometric amount (e.g. less than 1:1) of sodium formate may be used which makes isolation easier. In general, a pKa value less than about 4.2 (that of terephthalic acid) is needed to maintain the product as TA rather than terephthalic salts. If that is satisfied, at least one equivalence of water is needed as the reactant, and the inorganic component may be used at less excess than those for the bicarbonates and carbonates. In some embodiments, less than 1 equivalence of sodium formate relative to PET may be used. In an embodiment, other inorganic formate, other inorganic carboxylates may be used instead based on their melting point, pKa value of the respective conjugate acids, decomposition characteristics, and crystal structures. In an embodiment, the temperature profile, the back pressure, the screw design, etc. can all be adjusted to achieve the optimal conditions. In an embodiment, instead of water, other media, such as alcohol may be injected, in which case the inorganic alkoxide may be the reactive species and form terephthalic esters instead. In some embodiments, reactions with inorganic bicarbonate, carbonate, formate, or other carboxylate may instead be implemented in a ball mill or other instruments. In an embodiment, a mixture of 0.50 g of waste PET (which was precleaned and milled) and a slight excess NaHCO3 (2.5 equiv.) is ball-milled at a frequency of about 20 Hz to about 50 Hz, in a 10 mL jar using stainless-steel balls of 15 mm diameter for about 1 hour. The crude mixture is suspended in 20 mL of distilled water, and filtered. The filtrate is acidified with diluted HCl (1 M) until pH reaches 1. The precipitation is collected as TA. In an embodiment, the milling produces heat that may be utilized to decompose the NaHCO3. In an embodiment, the jar is adapted to include a heating jacket, thereby controlling the temperature, for example, at values described above.

In the above embodiment, the inorganic component does not directly implement a hydroxide, which reduces the hazardous profile of the processing, and reduces wear to the extruder and other equipment. In another embodiment, sodium hydroxide, potassium hydroxide, or other hydroxides may be implemented directly to mechanically react with PET. For example, at a stoichiometry of NaOH:PET:NaCl=1.1:1:1.5, the premilled mixture is fed into a twin-screw extruder equipped with the reverse conveyor segment and extruded. The product is received as terephthalic sodium, NaCl and ethylene glycol.

In an embodiment, the PET is a post-consumer resin (PCR) and has been washed (such as using an acidic wash or alkalinic wash). In an embodiment, the PCR and the inorganic bicarbonate are pre-mixed and ground in a shaker, mill, or any other suitable mixing device to achieve proper sizes and distribution. In an embodiment, the PCR may include other resin types or additives (e.g. halogen-type flame retardants). The milling may include milling with iron and SiO2 quartz sand or calcium oxide and SiO2 quartz sand to neutralize the halogens. In an embodiment, a cryogenic grinding step is conducted on the PCR to reduce its particle size so as to improve the efficacy of the premixing. In some embodiments, the cryogenic grinding, the premixing, or both are omitted. In an embodiment, the extruder may implement a single screw configuration with a gradually increasing section with a short kneading element in the final zone. For example, a temperature gradient of 30-220° C. may be used along the barrel and a screw speed of about 30 rpm, and a feed rate of 1 g/min to about 150 g/min may be implemented. In an embodiment, the extruder may implement a twin screw. For example, a screw design may consist of largely of conveying sections (with the channel depth decreasing from one conveying section to the next) and kneading segments containing combinations of 30°, 60° and 90° orientations of the segments, with greater angles applying the greater shear to the material. A screw speed of about 30 to about 250 rpm may be used. In some embodiments, if the screw speed is too large, the residence time may be too small for reaction to complete; if the screw speed is too short, mixing may not be sufficient. In some embodiments, the screw speed is about 50 rpm to about 150 rpm. In an embodiment, the screw speed is increased, and a reverse conveying component is included in the screw design, such as along with conveying and kneading segments. The reverse conveying segments retard the flow of the material through the extruder and thereby increasing the residence time. They may be strategically positioned after each kneading section, to hold the material in the high shear kneading sections for a prolonged period of time. In an embodiment, with the reverse conveying segment, the screw speed may be increased to about 120 rpm to 300 rpm with substantial residence time (such as about 2 min to 60 min). In an embodiment, the screw designs and parameters described here may be adjusted to improve contacts between the PCR and the inorganic bicarbonate.

In some embodiments, the mechanochemical recycling methods provided above are particularly suitable towards condensation polymers (such as polyesters, polyamides and the like). Accordingly, where PET is mixed with other types of polymers, such as polyethylene, polypropylene are not substantially degraded. In some embodiments, the recycling stream is first separated using any suitable conventional methods (such as floatation or the like) to remove those additional polymers. However, by changing the reaction conditions, similar chemical recycling may be provided to those other polymers. For example, if the mechanochemical reaction is conducted in an air environment, the mechanical chemical recycling degrades polystyrene into its monomers. Therefore, co-recycling may be achieved between PET and polystyrene. In some embodiments, the reaction is conducted in an enriched oxygen environment. The products of the mechanochemical recycling of PET and polystyrene are sufficiently different to allow easy separation at the end of reaction.

As described above, the present disclosure provides integrated methods for reducing wastes. In an embodiment, the PET (or other materials similar to PET in some relevant aspects) may be manufactured with or coextruded to include a radical scavenging component (including those with activators and therefore improved activities). In some embodiments, the PET (or similar) may be manufactured with end caps (e.g. silyl endcaps). In an embodiment, the degradation of the RS does not substantially cause property changes thereby not disrupting the recycling stream. In an embodiment, the silyl caps are cleaved in the normal mechanical recycling operations, such that recycled materials may be upgraded despite the initial incorporated protecting groups. In an embodiment, after repeated mechanical recycling, and when further mechanical recycling is no longer proper, the PET (or similar materials) may be chemically recycled using mechanochemical methods using extruders or ball mills to provide raw materials for the re-manufacturing of PET. Moreover, the PET recycled either chemically may be mixed with other plastics. Pre-treatment with mechanochemical methods may be implemented to remove, e.g. halogens. Moreover, mixed recycling streams may be tackled using a combination of conventional separations (e.g. floatation), pretreatment, adjusting the mechanochemical reaction conditions (e.g. with or without oxygen present), and post-recycling separations. Furthermore, the present disclosure also provides that the PET used for packaging applications may be configured to include the OS components as a way to extend shelf-life for the contents. As compared to other OS on the market, the ones described may be incorporated to the PET structure either as a copolymer, as an endcap, or coextruded, without producing small fragmented molecules that may cause toxicity concerns. Furthermore, by implementing the activator-bearing groups, oxygen scavenging catalysts may be avoided or reduced in amounts, such that any adverse effect to the recycling stream is minimized. Therefore, the present disclosure offers an integrated end-to-end solution for PET manufacturing, stabilization, application, mechanical recycling, chemical recycling that not only reduces plastics wastes, but also food, beverage, drug, or other materials wastes.

As described above, although the disclosure uses PET as an example, it is not so limited. However, some aspects of the present disclosure are more suitable to certain types polymers in certain circumstances. For example, the mechanochemical recycling method in absence of oxygen may be more suitable to polyesters, polyamides, and the like; the oxygen scavenging application may be more suitable to polyesters when appearance (e.g. transparency) is important. However, some aspects of the invention may be universally applicable. For example, the radical scavenging application for the purpose of mitigating thermal or light-initiated polymer degradation may be applied to all types of polymers.

In one aspect of the present disclosure, a RS is provided. The RS includes a first carbon atom directly attached to a hydrogen atom (H), and the first carbon atom further being directly attached to (1) each of a first group, a second group, and a third group, or (2) each of a strong mesomeric electron-donating group and a strong mesomeric electron withdrawing group (as defined therein). The first group includes a conjugated unit selected from a double bond, a triple bond, an aromatic ring. The first group further includes a first anchor atom. The first anchor atom has an sp² hybridization, an sp hybridization, or a lone pair of valence electrons. The first group is directly attached to the first carbon atom (C₁) at the first anchor atom. The second group includes a heteroatom and is selected from a triple bond, a C═N unit, a N═O unit, a first C═O unit directly attached to the first carbon atom and a second carbon atom, a second C═O unit directly attached to the first carbon atom and an oxygen, a third C═O unit directly attached to the first carbon atom and a first nitrogen atom (said first nitrogen atom being directly attached to a third carbon atom), a first fragment directly attached to the first carbon atom at an oxygen, a second fragment directly attached to the first carbon atom at a nitrogen, and a third fragment having at least three heteroatoms within a spatial distance of 4 A from the first carbon atom (the three heteroatoms includes a nitrogen)—provided that the second group is the third fragment if and only if the first group is an ester directly attached to the first carbon atom with an ester oxygen or an amide directly attached to the first carbon atom with an amide nitrogen. The third group is selected from a. hydrogen, an alkyl group, an aromatic group, a double bond, a triple bond, and a heteroatom. In an embodiment, the RS includes a N,N′-[1,3-phenylenebis(methylene)]bis(isoindolin), a N,N′-[1,3-phenylenebis(methylene)]bis(isoindolin-1-one), a poly[(1,3-phenylene)bis(methylene) adipamide], a poly(1,4-butadiene), a condensation product of a carboxylic acid) and poly(1,4-butyleneglycol) (sometimes referred to as poly(tetrahydrofuran)), a tribenzylamine, or a N,N,N′,N′-tetrabenzyl(1,3-phenylene)diamine. In some embodiments, the RS includes an activator moiety that is (1) directly bonded to the first carbon atom, (2) conjugatively connected, either directly or through multiple bonds, to the first carbon atom, (3) being in proximity (e.g. within two C—C bond distance, measured point-to-point in space) of the first carbon atom, or (4) being conjugatively attached to an A-H moiety, where A-H bond is a labile bond (such as having a bond dissociation energy less than 87.5 kcal/mol) and A is in proximity of the first carbon atom. In some embodiment, the activator is selected from a phenol having a phenol hydroxy group, a hydroxybenzotriazole having a hydroxy group attached to the triazole nitrogen, an N—Hydroxyphthalimide having the hydroxy group attached to the phthalimide nitrogen, an N—Hydroxyphthalimide with the hydroxy hydrogen substituted with an organophosphine, an amide with its amide nitrogen attached to a double bond or aromatic ring, an allyl having an allylic or a benzene having a benzylic C—H. In some embodiment, the RS includes a UV-absorbing moiety. In some embodiments, the RS includes an isocyanurate. In an embodiment, the OS is used in PET without the presence of an oxygen scavenging catalyst. Such catalysts are detrimental to the purpose of serving as a RS. In some embodiments, the first carbon atom is part of a ring structure. In some embodiments, the ring structure includes a heteroatom. In some embodiments, the first carbon atom is directly bonded to the heteroatom and another sp2 carbon. In some embodiments, the ring structure includes a 2,5-dihydrofuran motif, a 1,3-dihydro-2-benzofuran motif, an isoindoline motif, or a 2,5-dihydropyrrole motif In some embodiments, the ring structure includes a 1,3-dihydro-2H-inden-2-one motif, a cyclopent-3-en-1-one motif, or a cyclopenta-3-en-1-imine motif. In some embodiment, the ring structure includes a phthalide or an isoindolin-1-one. In some embodiments, the ring structure includes a lactone (e.g. 2-coumaranone) or lactam (e.g. oxindole). In some embodiments, the ring structure is a 6-member ring. In some embodiments, the ring structure is part of a polymeric backbone. In an embodiment, the RS includes a trivalent heteroatom (such as N or P), where the N is attached to at least one alkyl group. In some embodiments, the RS includes N,N,N′,N′-tetrabenzylethylenediamine. In some embodiments, the RS includes N,N′[1,3-phenylenebis(methylene)]bis(isoindolin). In some embodiments, an oxidation product of the RS includes a benzoate motif or a benzamide motif In some embodiments, an oxidation product of the RS includes an ethylene motif. In some embodiments, the RS includes two hydroxy end groups. In some embodiments, the RS is an oligomer, polymer, or dendrimer formed from any other the RS structures described above.

In one aspect, the present disclosure provides a polymer composition that includes the RS described above, and a polymer, in absence of a radical catalyst. In some embodiments, the polymer is a polyester, polyethylene, polypropylene, polystyrene, polyamide, or a polyurethane. In one aspect, the present disclosure provides an article formed from the polymer composition. In one aspect, the present disclosure provides a method of preparing an article using the polymer composition. In one aspect, the present disclosure provides a method for using the polymer composition to mitigate polymer degradation in recycling. In one aspect, the present disclosure provides a polymer composition that includes the RS described above, a polymer, and a radical catalyst. In some embodiments, the radical catalyst includes a manganese carboxylate. In some embodiments, the radical catalyst includes a mixture of a manganese carboxylate and a cobalt carboxylate. In some embodiments, the cobalt carboxylate is used at weight concentration less than 5 ppm. In some embodiments, the cobalt carboxylate is used at a weight concentration less than about 1 ppm. In some embodiments, the polymer composition is used as packaging. In some embodiments, the polymer composition is suitable for food contact. In some embodiments, the polymer composition is suitable for pharmaceutically acceptable. In one aspect, the present disclosure provides a method of preparing an article using the polymer composition. In one aspect, the present disclosure provides a method for preparing an article using the polymer composition to extend shelf-life of a material contained within the article. In one aspect, the present disclosure provides a method of reducing polymer degradation during processing and usage, the method including capping end groups of the polymers. In some embodiments, the method includes capping the end groups using a capping material that is removable under mechanical recycling conditions. In an embodiment, the method includes capping the end groups using a capping material that is removable using a dilute acid wash or a dilute alkaline wash. In an embodiment, the capping ligand is silyl capping groups. In an embodiment, the end groups capped are hydroxy groups. In an embodiment, the end groups capped are carboxylic grounds. In an embodiment, the end groups capped are amine groups. In an embodiment, the polymer is a polyester. In an embodiment, the polymer is a polyamide. In an embodiment, the capping group is a RS described above with a free hydroxy group and a silyl group. In an embodiment, the polymer is end-capped with a group that, upon oxidation, covers into an anhydride group. In an embodiment, the polymer is end-capped by a phthalan group. In an embodiment, the present disclosure provides a method for reducing degradation that includes end capping the end groups in a manufacturing process of the polymer. In an embodiment, the present disclosure provides a method of recycling which includes removing an end cap on the end groups of the polymer. In an embodiment, the present disclosure provides an integrated method that includes capping end groups of a polymer with a capping material during manufacture, providing the polymer for an application; receiving the polymer as a consumer-used resin, removing the capping material of the consumer-used resin during a recycling operation to form free end groups, inducing a condensation reaction between the freed end groups to form an upgraded resin, and reuse the upgraded resin for the application.

In one aspect, the present disclosure provides a method. The method includes treating a post-consumer resin (PCR) with a material capable of reacting with vinyl groups. In some embodiments, the material includes a double bond. In some embodiments, the material is itaconic acid. In some embodiments, the material is itaconic anhydride. In some embodiments, the material includes itaconic ester. In some embodiments, the material includes styrene. In some embodiments, the material includes a material capable of addition polymerization or ring-opening polymerization. In some embodiments, the material is alkylene oxide (e.g. ethylene oxide) or alkylene carbonate (e.g. ethylene carbonate). In some embodiments, the treating of the PCR includes treating with the material and an exchange catalyst. In some embodiments, the treating of the PCR includes treating with the material and an activator described above. In some embodiments, the treating of the PCR include forming a copolymer of the polymer and the material as a comonomer. In one aspect of the present disclosure, a method of recycling is provided. The method includes subjecting a polyester to a material with a hydroxide in a reaction under mechanochemical conditions. In some embodiments, the mechanochemical condition includes reactive extrusion. In some embodiments, the material decomposes into a gas product under the mechanochemical condition. In some embodiments, the material is an inorganic bicarbonate. In some embodiments, the reaction is conducted at a temperature above a decomposition temperature of the inorganic bicarbonate. In some embodiments, the inorganic bicarbonates is a sodium bicarbonate. In some embodiments, the reaction is conducted with the inorganic bicarbonate being at an excess. In some embodiments, the material is an inorganic carbonate with crystalline water in its structure. In some embodiments, the reaction includes injecting at least one equivalent of water relative to the polyester. In some embodiments, the reaction is conducted at a temperature above a glass transition temperature of the polymer. In some embodiments, the material is an inorganic salt of an acid having a pKa value less than about 4.2. In some embodiments, the material is an inorganic formate. In some embodiments, the reaction is conducted with the inorganic formate at less than 1 equivalence relative to the polymer. In some embodiments, the mechanochemical condition includes ball milling. In some embodiments, the reaction is conducted in an extruder. In some embodiments, the extruder is configured to include a reverse conveying segment. In some embodiments, the reverse conveying segments are positioned after each kneading section. In some embodiments, the reaction is conducted in absence of oxygen. In some embodiments, the polyester is PET. In some embodiments, instead of polyester, a polyamide is treated with the method described above. In some embodiments, instead of polyester, a polystyrene is treated with the method described above, in presence of oxygen.

In one aspect, the present disclosure provides a method, the method includes modifying the PET with a RS in the manufacturing of the PET. In some embodiments, the method includes modifying the PET with a RS in an extrusion process. In some embodiments, the method includes modifying the PET in a recycling operation. In some embodiments, the modifying the PET with a silyl end capping group in the manufacturing of the PET. In some embodiments, the modifying the PET with a silyl end capping group in the an extrusion operation. In some embodiments, the modifying the PET with a silyl end capping group in a recycling operation of the PET. In some embodiments, the method includes modifying PET with a material having a double bond during a mechanical recycling operation. In some embodiments, the modifying includes subjecting a vinyl end group of the PET to the material having the double bond. In some embodiments, the material includes itaconic component. In some embodiments, the material includes styrene. In some embodiments, the material includes a material capable of ring opening polymerization or addition polymerization reaction.

In one aspect, the present disclosure provides a method of integrated management of PET life cycle. The method includes modifying the PET in a manufacturing to reduce PET degradation in processing and use, such as by introducing RSs or end capping groups. The method also includes mechanically recycling the PET, and chemically recycling the mechanically recycled PET chemically, such as using mechanochemical methods. In some embodiments, the mechanically recycling of the PET includes introducing a RS. In some embodiments, the mechanically recycling of the PET includes introducing a silyl end capping group. In some embodiments, the mechanically recycling of the PET includes introducing a material with a double bond (e.g. itaconic acid, anhydride or ester, or styrene, or alkylene oxide, or alkylene carbonate) to react with a vinyl end group of the PET. In some embodiments, the chemically recycling includes pre-treating a mixture of the PET with another polymer including halogens using mechanochemical milling with an inorganic bicarbonate or carbonate.

As described above, this present disclosure provides method to recycle certain waste materials using chemical methods. Importantly, it has generally been accepted that chemical recycling is cost ineffective, such that it is not a feasible route for large scale recycling. Indeed, studies have shown that chemically recycling polyethylene terephthalate, polyolefins, polyvinylchloride, etc. into their respective raw materials are expensive, sometimes more expensive than the virgin materials. However, such argument ignores the important advantage chemical recycling has over mechanical recycling—that is, the end product need not be the raw materials, but rather a value-added product. Moreover, the chemical recycling may utilize raws that are no longer proper for mechanical recycling (such as when the mechanical strength has deteriorated so much that any further processing will cause the recycled material to be unsuitable for the intended purpose; or when the raw include dark colors or otherwise incompatible additives or degradants that cannot be easily removed in mechanical processes). This present disclosure provides methods that chemically recycle wastes into value-added products, such that it becomes economically viable. In other words, the advantages provided by this present disclosure includes at least two aspects, that is (1) environmental benefit, and (2) economic benefits. Moreover, although the description below describes various starting materials as “waste,” “after-use” “after consumer” “trash” “garbage” or various similar names, the disclosure also describe novel methods of synthesis that may be applied to the underlying material, regardless whether they are virgin material, scraps, consumer used, industrially used, etc.

The target material for recycling may be any suitable waste materials. For example, this may be municipal wastes, such as municipal solid wastes (MSWs). MSWs include paper, plastics, metal, yard trimmings, wood, glasses, etc. Generally, MSWs or other similar waste streams may be processed at local facility that roughly sort these different types of wastes into different categories. Afterwards, each category may be further processed for mechanical recycling or landfill. Some embodiments described below may benefit from such crude separations. However, some other embodiments described below may require simpler separations (such as into fewer categories) or may require no separation at all. Any conventional separation methods may be used. Article titled “Techniques for separation of plastic wastes” by Silvia Serranti and Giuseppe Bonifazi (last accessed on Jul. 11, 2020 at https://doi.org/10.1016/B978-0-08-102676-2.00002-5) describe some such methods, and is hereby incorporated by reference in its entirety. However, other plastics separation methods beyond the scope of this paper may also be used. For example, in some embodiments, separation of PET and PVC may be required, and surface modification of PVC using potassium permanganate may be used.

In some embodiments, the waste resins may undergo a washing process. Any suitable washing liquid may be used. This may include caustic washing, water washing, etc. Particularly, in this example, an amine solvent (such as benzylamine) is used as the washing solvent. The waste resins are soaked in a minimal amount of the washing solvent (that sufficiently cover all waste resins) for a suitable period of time, for example about 12 hours to about 48 hours. The time required may be dependent upon the nature of the colorants involved. In some embodiments, heating may improve the extraction efficiency. The colorants and/or other additives embedded in the waste resins are extracted into the washing solvent. The washing solvent is separated by any suitable methods, such as decantation, filtration, centrifuge, etc. The extracted colorants and/or additives may be recovered using any suitable methods, such as distillation, chromatography, other suitable methods, or combinations thereof. In some embodiments, the distillation of the washing solvent (e.g., benzylamine) may be coupled to the chemical recycling methods such that the minimal extra energy may be required to recover the colorants and/or the washing solvent. In some embodiments, as described in more detail later, a portion of the waste resin may be dissolved in the washing solvent (such as benzylamine) by physical interaction or chemical interactions. In some embodiments, this process modifies the physical appearance of the waste resin. Other washing solvent may also be used depending on the solubility of the resin, the colorants, other additives or impurities in the solvent. In some embodiments, the waste resin may be subject to an amorphization (that is, to make the resin amorphous rather than crystalline) process prior to the washing process. For example, the waste resin may be subject to an extrusion process followed by a quenching process. For example, reins that include large amount of crystallinity may benefit from this treatment. Alternatively, the waste resin may be heated to melt and quenched rapidly. In some embodiments, the reaction conditions described herein may be carried out using reactive extrusion, such as those described by the several reactive extrusion books incorporated by reference through the '209 application.

Polyolefins may be chemically recycled using gasification, pyrolysis processes, or similar processes to generate alternative fuels. There have been a lot of research and commercialization effort in this area with limited success, primarily due to unfavorable economics. A larger portion of the cost associated with such separation process is the initial separation, as well as the lack of value derived from its co-existing other waste materials. The methods provided by this disclosure imparts value to the co-existing wastes, and indirectly improves the economics for these processes of polyolefin. In some embodiments, the fuel generated from such processes may be used to power or partially power the facility used in the chemical recycling operation, for example, for the operation of other recycling processes described herein. Additionally, as described later, the disclosure provides methods that allow valorization of polyolefins along with other waste materials into activated carbon.

In some embodiments, Polyvinylidene chloride (PVC) and Polyvinylidene dichloride (PVDC) may be chemically recycled. Although this present disclosure refers mostly to PVC, similar methods may be implemented for PVDC or other similar resins. In some embodiments, PVC and PVDC may be hydrolyzed partially or completely into polyvinyl alcohol. For example, the hydrolysis may be using sodium hydroxide aqueous solution. In some embodiments, the partially or completely hydrolyzed PVC/PVDC may be subject to any of the following treatments. The presence of hydroxy groups, and the amounts thereof may adjust a hydrophilicity (or hydrophobicity) of the polymer, and adjust the affinity of the material towards other chemicals. As described later, this avails the hydrolyzed PVC/PVDC as a chemical absorption or adsorption material. The chemical that may be absorbed or adsorbed may be spilled oil, spilled organic molecules, hazardous materials, or any other suitable materials. These products are referred to as PVC-based absorption materials. With respect to the pollution control market, alone, the estimated market value was previously estimated at about $125 billion in 2020. The methods provided herein utilize a pollutant (e.g. waste plastics) to form a pollution control material.

In some embodiments, the PVC-based absorption materials may be partially or completely aminated PVC. For example, it may be PVC with pending aromatic groups. The PVC is aminated with an organic amine. For example, a mixture of PVC (1 eq.) and p-toluidine (or aniline, or diphenylamine, 2.25 eq.) is stirred at 180° C. for about an hour. The product is cooled off and dissolved in 1,2-diehloroethane. After filtering off the byproduct and excess starting material, the filtrate is collected and precipitated with methanol into a light-grey powder. In some embodiments, a dry flow of hydrogen chloride is introduced into the filtrate (before drying or precipitation) until no more hydrochlorides form. The resulting hydrochlorides are treated with water to afford partially aminated PVC after filtration. The filtrate is further vacuum evaporated on a water bath to generate completely aminated PVC as a red crystalline product. The PVC-based absorption materials have chlorine functional groups, amine functional groups as well as aromatic functional groups. Other amines may alternatively used, such as benzylamine, etc.

In some embodiments, PVC is treated using a diamine, such as ethylene diamine. For example, PVC is reacted with an excess amount of ethylenediamine at about 80° C. for overnight. The ethylenediamine may be neat or at about 50% to about 100% in aqueous solution. The temperature may be maintained using an oven, oil bath, or any other suitable methods. Agitation may be maintained during the reaction period. At the end of the reaction, the resin is separated (such as decanted, filtered, centrifuged, etc.) and washed with large amounts of water. The product is subsequently dried (such as air dried). This PVC-based absorption material includes increased porosity as compared to materials generated from amination using monoamines. Those large pores may be used to absorb larger organic molecules (such as pollutants).

In some embodiments, PVC is treated with polyethylene glycol to form PEG-grafted PVC. For example, aminated PVC of the previous embodiment (1 g) is dissolved in 25 ml dry THF in a 100 ml round-bottomed flask, 2 ml of Bis(4-isocyanatocyclohexyl)methane (HMDI) is added, stoppered and the mixture is stirred magnetically for 1 h at room temperature. The resultant product is precipitated in dry hexane and washed with the same solvent over a fluted filter paper many times to remove the excess HMDI. The product is re-dissolved in THF and treated with 2.0 g of PEG for 15 min under magnetic stirring at room temperature. After the reaction, the product is precipitated in ethanol and extensively washed with the same solvent to remove the unreacted PEG and dried in vacuum. This PVC-based absorption material includes different functional groups and may therefore have different affinity to different chemicals that may need to be absorbed or adsorbed.

In some embodiments, the diamine-treated PVC, the PEG-treated PVC may be further treated, for example with diacid, anhydride, or any other suitable ditopic organic molecules to link the amine or hydroxy groups together to form even larger pores. This process produces derivatized PVC with large voids in its structures, having amine and/or ether groups. Not only that the larger voids may be used to absorb larger organic molecules, the hierarchical structure may impart in the modified PVC more surface area and volume that may improve the efficacy of its use in chemical control or pollution control applications.

The method described above provide one way to form large pores in a recycled PVC structure. Alternatively, PVC or PVDC may be dissolved, or melted and reacted with stoichiometric amount of diamine or diols to directly form the product having larger voids. In some embodiments, the identity of the diamine, diol, the linking group, etc. may be selected to form voids having different sizes and different affinities. In some embodiments, the linking groups may include additional functional groups, such as aromatics, carboxylic groups, etc. These sites provide affinity to different pollutants. As described, the linking group or pendant groups may include benzene rings. Such derivatized PVCs have large affinity towards aromatics compounds. Accordingly, they may be used to absorb aromatic pollutants from, from example, oil spills on the sea, or serve as chemical spill pads, etc. In some embodiments, the linking groups may include longer chains. Such voids formed include flexibilities. Accordingly, the product has expandability after absorbing the pollutants relative to its original size.

In some embodiments, the PVC-absorbing materials may include aromatic functional groups. Once the pollutant (or chemical) absorption is completed, the material may be subject to light treatments (such as UV-light treatment). Such treatments may be adjusted to either cause degradation of the absorbed and/or adsorbed material along with the PVC-based absorbing material, (in which case, concentrated pollutants are collectively oxidatively degraded along with the PVC-based absorption material) or, it may be adjusted such that only the PVC backbone is degraded, thereby releasing certain useful pollutants, such as aromatics. Meanwhile, the degradation of the PVC backbone may form polyvinyl alcohol, which may be used for various applications. In some embodiments, the diamine used to treat the PVC is benzene diamine; or xylylene diamine.

In some embodiments, rather than using linking groups, the aminated PVC may be reacted with carrier (or matrix) materials, such as zeolites, metal-organic frameworks, etc. to form a distributed layer of the porous material. This may be combined with the linked polymers, to form tiered porous structures for improved efficiency.

Although the disclosure in this section describes PET as the waste plastics being processed, other polymers having similar properties may similarly be processed. For example, for aminolysis treatment described herein (such as with benzylamine to form OS), other polyesters (polybutylene terephthalate, polylactate, polyglycolide, other polyesters such as NPG neopentylglycol-based polyesters, CHDM-based polyesters, polyesteramide, polycarbonate, etc.) may similarly treated. Moreover, polyamides and polyurethanes may also similarly treated. In some embodiments, the treatment of the polyamides may benefit from the presence of exchange catalysts, although not always necessary. Additionally, similar treatments may be conducted on polyacrylates, with the reactions occur on the side chains rather than the polymer backbones. This may be beneficial for certain applications (such as when the target material requires the maintenance of the polymer backbone).

In some embodiments, PET may be chemically recycled into phthalides. Phthalide is a class of molecule particularly important as a drug intermediate. For example, PET first undergoes aminolysis with diethylamine to form N,N,N′,N′-tetraethyl-p-xylylenediamide. The N,N,N′,N′-tetraethyl-p-xylylenediamide is reacted with sec-butyllithium-tetramethylethylenediamine (TMEDA) in tetrahydrofuran at −78° C. for 1 h, followed by addition of paraformaldehyde to form 5-Diethylcarbamoylphthalide. Alternatively, the N,N,N′,N′-tetraethyl-p-xylylenediamide is reacted with 2 equivalents of ethylene bearing an electron-withdrawing group to form substituted phthalide motifs. For example, the N,N,N′,N′-tetraethyl-p-xylylenediamide (0.1 mmol) is mixed with [RhCp*Cl₂]₂ (0.005 mmol) and Copper acetate Cu(OAc)₂.H₂O (0.4 mmol) in a 3:1 mixture of ethylenedichloride and acetic acid solution (1 mL). Ethyl acrylate (0.4 mmol) is introduced under air at ambient temperature. The reaction mixture is stirred at 130° C. for 21 h. The reaction mixture is diluted with ethyl acetate (10 mL) and washed with water to afford the 3,7-diethoxycarbonylfurano[3,4-f]isobenzofuran-1,5-dione. In some embodiments, furano[3,4-f]isobenzofuran-1,5-dione may similarly formed using formaldehyde.

In some embodiments, PET may be converted into phthalide derivatives, such as 5-carboxyphthalide, 5-cyano-phthalide, or further into 3,7-dicarboxy furano[3,4-f]isobenzofuran-1,5-dione or 3,7-dicyanofurano[3,4-f]isobenzofuran-1,5-dione. For example, PET reacts with 1.3 equivalents of paraformaldehyde in fuming sulfuric acid (with about 20% to 25% SO₃) at a temperature of about 125° C. to about 150° C. for about 4 to about 20 hours. The product is cooled and quenched with ethanol. After workup, 5-ethoxycarbonylphthalide is received as the product, which may undergo hydrolysis to form 5-carboxyphthalide, or undergo aminolysis with ammonia in methanol at refluxing temperature to form 5-carbamoylphthalide. Alternatively, PET may be extruded with paraformaldehyde in presence of a lewis acid, such as zinc chloride, under reactive extrusion condition, to form 5-carboxyphthalide. The 5-carbamoylphthalide may be converted into 5-cyanophthalide by a dehydration reaction with thionyl chloride (1.5 eq.) and a dimethylformamide catalyst in toluene and to receive the product as a precipitation. In some embodiments, the 5-carboxyphthalide may be converted into 5-chlorocarbonylphthalide which may be further derivatized into 5-carbonylphthalide, and further intermediates and products common in pharmaceutical or other chemical processes.

All these phthalides are important pharmaceutical intermediates. For example, 5-cyanophthalide may be converted into the active ingredient of, for example, the antidepressant drugs Lexapro and Celexa. The global antidepressants market for the grew from $14.3 billion in 2019 to about $28.6 billion in 2020. Moreover, phthalide derivatives are also common intermediates for dyes, resins, pesticides, and other drugs. As of Jul. 13, 2020, polyethylene terephthalate is priced at $339.2/kg at Sigma Aldrich; while 5-carboxyphthalide is priced at $806.0 per gram; and 5-cyanophthalide is priced at $4,680/kg.

In some embodiments, 5-carboxyphthalide or 5-ethoxycarbonylphthalide undergoes a decarboxylation reaction (such as thermal decarboxylation or oxidative decarboxylation) to produce phthalide. Phthalide is a raw material for oxygen scavenger, m-xylylene-bis(phthalimidine). For example, phthalide and m-xylylenediamine is reacted in neat condition in a Dean-Stark apparatus for overnight under stirring to form the m-xylylene-bis(phthalimidine). In some embodiments, 5-ethoxycarbonylphthalide is reacted with m-xylylenediamine at a similar condition to form a polymer version of the m-xylylene-bis(phthalimidine). There has been a need for mitigating the migration of oxygen scavenger molecules or degradants thereof in the polymer matrix for food, beverage, and pharmaceutical applications. Accordingly, the polymer version may be preferable for the purpose of reducing migrations and/or reducing small molecular degradants. In some embodiments, furano[3,4-f]isobenzofuran-1,5-dione may be used in place of phthalide to form a different form of polymeric version. In some embodiments, 5-cyanophthalide may be employed to first form the m-xylylene-bis(5-cyanophthalimidine). The additional cyano group may be used to further incorporate other functional groups and/or extend the molecular structure. For example, the cyano group may react with an amine or diamine under Grignard condition to form amides. In some embodiments, n-butyl-aminomagnesium bromide is prepared by dropwise addition of n-butylamine (42.7 mmol) in 5 mLTHF to a stirred solution of ethylmagnesium bromide(38.8 mmol, 12.8 mL of 3 M solution in ether) under nitrogen. The mixture is stirred for 1 h at 30° C. The resulting magnesium amide solution is added to a stirred solution of m-xylylene-bis(5-cyanophthalimidine) (4.85 mmol) dissolved in THF (2 mL) and the mixture is stirred for 1 h at 30° C. The reaction is quenched by adding to a stirred mixture of dichloromethane (20 mL) and aqueous HCl (10 mL of 2 M solution) at 22° C. The organic layer containing the product is worked up to provide the product linked to an amide. For example, the amide may be hydrolyzed into a carboxy group which may be used to tether an activator, another functional group, or to a catalyst.

In some embodiments, PET is converted to p-xylylene glycol. For example, a portion of the PET first undergoes catalytic glycolysis, for example, at 190° C. to form bis-2-hydroxyethyl terephthalate (BHET). Additional PET is introduced and dissolved in the BHET at 190° C. under agitation. The system is purged with several cycles of nitrogen gas. Polymethylhydrosiloxane (PMHS, 6 eq. based on total PET starting material) and Tetramethylammonium fluoride (TBAF, 0.1 eq.) is introduced into the system while maintaining a constant nitrogen purge flow. The reaction mixture is stirred for 36 hours. At the end of the reaction, the temperature is allowed to cool down. A methanol solution of sodium hydroxide is added and stirred for 2 hours to form p-xylylene glycol.

In some embodiments, p-xylylene glycol forms a component for OS. Additionally, for example, p-xylylene glycol may react with carboxylic acids to form other OS. Furthermore, p-xylylene glycol may be converted into p-xylylene diamine, which may be used for oxygen scavenger applications, and a variety of other applications, such as in the production of thermally stable polyamide fibers such as Kevlar. Conventional synthesis of p-xylylene diamine requires ammoxidizing xylene to phthalonitrile (˜400° C.), followed by hydrogenation (100 bar H₂). In some embodiments, p-xylylene glycol (0.5 mmol) is mixed with aqueous NH3 (25%, 2 mL), t-amyl alcohol (2 mL), and Raney Ni (200 mg). The mixture is heated at about 160° C. to about 180° C. for about 18 hours to provide p-xylylene diamine. In some embodiments, the p-xylylene glycol and/or p-xylylene diamine may be used to produce OS, such as those described in U.S. Pat. No. 10,316,167. In some embodiments, p-xylylene glycol is reacted with urea to convert to an oxygen scavenger. For example, into p-xylylene glycol (either virgin or prepared from the PET waste) is added dibutyloxotin at a molar concentration of about 0.05% to about 2%. 0.9-1.0 equivalents of urea is added into the reaction system slowly under reduced pressure to form the product, along with ammonia gas as a byproduct. The ammonia byproduct is absorbed using an ammonia trapper and removed from the reaction system. The reaction is conducted at about 100-250° C. and maintained at about 2 hours to about 18 hours. The product is an oxygen scavenger. Moreover, the product has hydroxy terminals and may be used to be tethered onto polymer (such as PET) backbones, incorporate activator groups, incorporate additional functional groups, other polymerize itself. In some embodiments, the urea is added at 0.45-0.50 equivalents instead. The product possesses large pores and may be used similar to PVC derivatives described above in pollution mitigations. Similarly, the product has hydroxy end groups and may be further derivatized. This is also an oxygen scavenger. In some embodiments, p-xylylene glycol may react with benzylamine, or p-xylylene diamine may react with benzyl alcohol to form imine products, which are OS. For example, 50 mg 3 wt % Au/ZrO₂ catalyst, p-xylylene glycol (0.25 mmol), benzylamines (0.5 mmol), toluene (10 mL) and potassium methoxide (0.5 mmol) are charged into a flask under oxygen atmosphere. The suspension is stirred at 60 C for 24 h to form N,N′-dibenzylidenexylylene diamine. This is an oxygen scavenger. In some embodiments, same reaction condition is adopted except that 0.25 mmol of p-xylylenediamine is used in place of the benzylamine. Accordingly, a polymeric version of the N,N′-dibenzylidenexylylene diamine is received, which is also an oxygen scavenger.

In some embodiments, PET is dissolved in THF and reduced using Urushibara nickel (Al—NiCl₂) to produce terephthalaldehyde (or p-phthalaldehyde). In some embodiments, the p-phththaldehyde is reacted in presence of aluminum isopropoxide under mild reaction conditions to form polyterephthalaldehyde. In some embodiments, terephthalaldehyde reacts in presence of a catalytic amount (2 mol %) of aluminum isopropoxide at about 30° C. in methylene chloride (10 mL for 1.0 g of terephthalaldehyde) for 18 hours to form polyterephthalaldehyde. The polyterephthalaldehyde is an oxygen scavenger. In some embodiments, the polyterephthalaldehyde is reacted with amines to further form different amide-based OS. In some embodiments, polyterephthalaldehyde is treated with amines to form smaller size OS. For example, polyterephthalaldehyde may be reacted with benzylamine or dibenzylamine. In some embodiments, the polyterephthalaldehyde may be treated with diamines, thereby forming a polymeric network. In some embodiments, m-xylylenediamine reacting with polyterephthalaldehyde results in poly(m-xylyleneterephthalamide). In some embodiments, rather than m-xylylene diamine, p-xylylene diamine produced according to methods above from waste PET may be used.

In some embodiments, the poly(m-xylyleneterephthalamide) so formed includes a network (rather than linear polymeric) having multiple benzylbenzamide and N,N′-dibenzylbenzamide fragments, along with large pores amongst those fragments. In some embodiments, those pores may be useful for pollution mitigation, similar to the PVC-derived porous frameworks described above. For example, the large pores include benzene groups, amide groups, hydroxy end groups, amine end groups, carboxyl acid end groups, etc. They each attract different organic molecules (such as organic pollutants). In some embodiments, the polyterephthalaldehyde may instead be treated with other diamines, such as hexamethylenediamine, or other derivatized diamines, triamines, or polyamines. This allows the pores of the subsequently formed network to be of different sizes, and/or include other functional groups, or a variety of functional groups. Therefore, the pollution control products formed therefrom may either be a general application pollutant mitigation product, or be a specific application pollutant mitigation product (e.g. absorbing a particular type of pollutants with selectivity).

In some embodiments, benzylbenzamide or polymeric/oligomeric molecules having such fragments are reduced such that the amide group (—(C═O)—NR—) is reduced into amine groups (—CH₂NR—). Any suitable method may be used, such as using methods reviewed. In some embodiments, PMHS or other silanes may be used. For example, the amide (or polyamide) starting material (1.0 mmol) is charged into an oven dried Schlenk tube. [Fe₃(CO)₁₂] (0.02-0.1 mmol) is added and well mixed. PMHS (4.0-8.0 mmol) and dry toluene or dibutylether (3 mL) is added respectively after purging the Schlenk tube with nitrogen. The mixture is stirred at 100C for 1 day. The reaction mass is filtered through Celite and worked up to provide essentially a polymer having a plurality of benzylamine functionality. The product is also an oxygen scavenger. Moreover, the product may also absorb organic molecules like pollutants, similar to those described above but with affinities that may be different. In some embodiments, the PMHS used in the above and following reactions may be a waste byproduct from the silicon industry. Accordingly, sustainability value of the methods described here are further enhanced.

In some embodiments, PET undergoes aminolysis reaction or ammonolysis reaction to form terephthalamide. In some embodiments, the terephthalamide undergoes Hoffman rearrangement with bromine and sodium hydroxide into p-phenylenediamine. P-phenylenediamine is widely used in almost all hair dye formulations, and is also used as a photographic developing agent and as an intermediate in the manufacture of azo dyes, antioxidants, and accelerators for rubber vulcanization.

In some embodiments, waste PET may be treated with an excess of allylamine at 170° C. under pressure of 2 MPa for 2 h to provide N,N′-bisallyl terephthalamide. This is an oxygen scavenger. In some embodiments, the allylamine is limited (e.g. the molar amount of allylamine is less than twice the molar amount of PET monomer in the PET). Accordingly, the PET is not entirely degraded by the aminolysis reaction. Accordingly, PET fragments are maintained. In other words, the product includes a PET fragment with an allyl group appended as an end group. For example, the PET now have a modified end group fragment that includes -Ph-C(═O)—O—CH₂—CH₂—O—C(═O)-Ph-C(═O)—NH—CH₂—CH═CH₂. In some embodiments, the allylamine may be derivatized, and the modified end group fragment may include -Ph-C(═O)—O—CH₂—CH₂—O—C(═O)-Ph-C(═O)—NH—CHR₁—CR₂═CR₃R₄, where R₁˜R₄ may each be independently selected from H or any organic residues. As described earlier, this is an oxygen scavenger. Being tethered to the polymer or oligomer fragment improves the stability of the end group, such that, migration of these molecular fragments may be mitigated. In some embodiments, rather than allylamine, benzylamine or dibenzylamine may be used as the aminolysis material. Accordingly, in some embodiments, the product includes a PET fragment with a benzyl group appended as a modified end group. For example, the modified end group may include -Ph-C(═O)—O—CH₂—CH₂—O—C(═O)-Ph-C(═O)—NH—CHR₁-Ph, where Ph may be derivatized. In some embodiments, the amount of amine is selected to be between twice the molar amount of PET monomer in the PET, and the amount of PET chains in the PET. Accordingly, in some embodiments, the amine-treated PET may include a PET fragment having two modified end groups as described above. In some embodiments, the amount of PET chains in the PET may be calculated from the weight of the PET used and the number average molar molecular weight (Mn) of the PET. In some embodiments, the modified PET may be used directly as a masterbatch for a PET extrusion process or an extrusion process for a different matrix resin. Accordingly, the amount of amine to be used to treat the PET may be calculated based on the expected dosage of the oxygen scavenger.

In some embodiments, PET is similarly treated with xylylenediamine (such as those produced from recycling PET described above) to form a network of modified PET. The modified PET includes benzylbenzamide functionality and is an oxygen scavenger. In some embodiments, it may be important to maintain or increase the molecular weight of the modified PET. Moreover, the modified PET includes large pores and may be used as an organic (or pollutant) absorption material. In some embodiments, the waste PET may be modified in a conventional organic reaction apparatus. In some embodiments, the waste PET may be modified using reactive extrusion methods using an extruder. In some embodiments, carriers may be used to facilitate the distribution of the starting materials (such as amines). In some embodiments, the modification of PET described in the present disclosure may include using an exchange catalyst.

In some embodiments, certain aminolysis reactions of PET may produce sticky materials and/or encounter diffusion restriction issues due to the size of starting materials. As a result, the reaction may be sluggish. This may be circumvented by using a two-step reaction, where the PET first undergoes alcoholysis or aminolysis with small alcohols, ammonia, or amines. Subsequently (or simultaneously in certain circumstances), the product undergoes an exchange reaction with diamines (such as ethylenediamine or p-xylylene diamine) to form the desired product having xylylene diamine functionalities and/or the large pores. In some embodiments, an exchange catalyst may be used, which may be nano zeolite beta, chitosan, transition metal alkoxides, oxides, or carboxylates (such as those of Ti, Zr, etc.). In some embodiments, the use of the exchange catalyst activated PET and allows reduction of reaction temperature.

In some embodiments, PET may be treated with polyalkylene glycol in presence of an exchange catalyst. The transesterification reaction may because the polyalkylene to be tethered to the carboxylic group of the PET. In some embodiments, the polyalkyene is polyethylene glycol, polypropylene glycol, polybutylene glycol, or polypentylene glycol. In some embodiments, the derivatized PET includes a modified end group having -Ph-C(═O)—O—CH₂—CH₂—O—C(═O)-Ph-C(═O)—O—((CH₂)ₙ—O)ₘ—H, where n may be about 2 to about 6, and m may be any integer greater than 2. This product is an oxygen scavenger. In some embodiments, in addition to the transesterification catalyst, zinc (Zn) salt, aluminum (Al) salt, zirconium (Zr) salt, titanium (Ti) salt, etc. is also included to enhance the activity. In some embodiments, the PET may be treated with polyalkylenimine. For example, the PET may be treated with polyethylenimine (or polyaziridine). This forms -Ph-C(═O)—O—CH₂—CH₂—O—C(═O)-Ph-C(═O)—N—CH₂—CH₂—N)ₘ—H. For example, the PET may be treated with polybutylenimine. This forms -Ph-C(═O)—O—CH₂—CH₂—O—C(═O)-Ph-C(═O)—N—(CH₂)₄—N)ₘ—H. In some embodiments, these reactions may include using an exchange catalyst. This is an oxygen scavenger. In some embodiments, the PET may be treated with polyetheramine. Polyether amine may be tethered onto the PET backbone by methods similar to those described above.

In some embodiments, the recycled PET may be slightly yellow-colored. In some embodiments, this indicated the presence of certain amount of double bond end groups. These double bond end groups may be used to tether functional groups, such as those described as OS. However, other functional groups may similarly be tethered. In some embodiments, an amine molecule is used to treat the recycled PET, which then undergoes hydroamination reaction to be tethered on to the end groups. As compared to the method described above, this method may require a different reaction condition. For example, rather than the exchange catalyst described earlier, the reaction instead requires a hydroamination catalyst, such as catalysts based on lithium, calcium, aluminum, indium, bismuth, transition metals such as lanthanides, as well as zeolites. In some embodiments, a benzylamine or allylamine is used to treat the recycled PET. The recycled PET may have a vinyl end group, such as —C(═O)-Ph-C(═O)—O—CH═CH₂. Accordingly, the treated PET may have a modified end group including —C(═O)-Ph-C(═O)—O—(CH₂)₂NHCH₂Ph or —C(═O)-Ph-C(═O)—O—(CH₂)₂NHCH₂CH═CH₂. This is another method to tether oxygen scavenger or other functional groups to the recycled PET to impart functionality. Similarly, in addition to amines, other functional groups, such as carboxylic group, other double bonds, etc. also react with double bond to cause tethering. Accordingly, OS having carboxy groups, double bonds (except those forming part of the oxygen scavenging moiety, such as those attached to C₁ atom), may be tethered to the recycled PET this way. In other words, oxygen scavenging molecules described here (or those of the '209 application) may be derivatized to include an amine group, a carboxylic group, a double bond separate from the C₁ atom, and causing those groups to react with the double bond end groups of PET to cause tethering.

Other recycled plastics may similarly include double bond end groups, and therefore may be similarly derivatized to form end-capped OS. For example, polyolefins such as HDPE, LDPE, Polystyrene, polylactic acid, polyurethane, polycarbonate, among others. Any OS known to react with a double bond may be tethered onto any polymers that forms double bond end groups either natively or as part of degradations. Accordingly, these recycled (or virgin) resins may be treated to impart oxygen scavenging functionalities. Polymer compatibility is often a concern for packaging appearance, mechanical strengths, etc. Mixing in a different polymer as an additive often leads to such incompatibility and degrade those properties. Using the method here allows the preparation of OS appended polymers for that specific polymer (such as oxygen scavenger-appended polypropylene to be used with polypropylene), thereby entirely remove the incompatibility concern. For example, recycled polyethylene may include double bond end groups. Benzylamine or dibenzylamine may be used to treat the polyethylene such that the end group includes —CH₂—CH₂—CH₂—CH₂—NH—CH₂-Ph, or —CH₂—CH₂—CH₂—CH₂—NH—CH(Ph)-Ph. Alternatively, allyl amine may similarly used to form polyethylene with end groups having —CH₂—CH₂—CH₂—CH₂—NH—CH₂—CH═CH₂, or —CH₂—CH₂—CH₂—CH₂—NH—CH₂—CH═CH₂. Similarly, polypropylene may be treated to form products having —CH₂—CH(CH₃)—CH₂—CH(CH₃)—NH—CH₂—CH═CH₂, —CH₂—CH(CH₃)—CH₂—CH(CH₃)—NH—CH₂—CH═CH₂, —CH₂—CH(CH₃)—CH₂—CH(CH₃)—NH—CH₂-Ph, or —CH₂—CH(CH₃)—CH₂—CH(CH₃)—NH—CH₂-Ph.

The various methods described above may be used to chemically recycle other waste plastics thereby providing value-added products. For example, similar to the aminolysis of PET, polylactic acid may be aminolyzed with benzylamine to provide N-benzyl-2-hydroxypropionamide, which is a pharmaceutical intermediate. Polyacrylates (such as poly(methyl acrylate), poly(ethyl acrylate), poly(methyl methacrylate), etc.) may be aminolyzed into a polyethylene or polypropylene backbone with partially or completely derivatized side chain that includes benzyl amide functionality. These are OS that may be used for polyolefin applications without concern for incompatibilities. In some embodiments, diamines may be used to form a polymeric framework that has large pores suitable for chemical and/or pollution control. Polycarbonate and polyurethane may similarly treated to form OS suitable to improves their respective properties.

Moreover, the various methods described above may be used to chemically recycle other waste materials than plastics. For example, itaconic acid may be produced from sugarcane bagasse. In some embodiments, the itaconic acid may be reacted with the amino end groups of polyamides and/or polyamines, polyetheramines, etc. in order to tether the itaconic acid (or esters thereof) onto the polymer backbone. The itaconic acid (or esters thereof) functions as the oxygen scavenger. In some embodiments, the oxygen scavenger may be a polyamide formed from itaconic acid and diamines (such as hexamethylenediamine or xylylene diamine). For the case of xylylenediamine product with itaconic acid, the activity of the product may be higher than, for example, adipic acid. In some embodiments, itaconic acid (1 eq.) and m-xylylenediamine (0.48 eq.) is reacted for 2 hours at 100° C. in water (400 mL water for each 1 eq.). A second batch of m-xylylenediamine (0.48eq) is then added and the reaction temperature is increased to about 270° C. Water is allowed to be distilled out. The product is discharged as a melt and subsequently cooled to provide the itaconic-xylxylene diamine polymer.

As described above, colorants (such as dyes and pigments) may be dissolved in the pre-treatment washing solvent (such as benzylamine). These colorants may be recovered by distilling off the washing solvent. This process may be coupled with the other reactions described herein, for example, with the introduction of benzylamine into another reaction that utilizes benzylamine as a starting material, so as to reduce the energy footprint of the processes. In some embodiments, the recovered dyes or pigments may be used as colorant additives for subsequent processing. In some embodiments, the recovered dyes or pigments may be anthraquinone-based. In some embodiments, the recovered anthraquinone colorant may be used a textile additive which has antimicrobial functionality. For example, the anthraquinone colorant may absorb UV-light and generate reactive species that kills the microbes.

In some embodiments, anthraquinone molecules may react with the benzylamine to form OS. For example, after the washed resins are isolated, the liquid portion including the benzylamine and the anthraquinone are adjusted to a pH value of about 5 and heated to reflux condition under inert atmosphere for 2-18 hours using a Dean-Stark setup, such that condensation water is removed. The product is an oxygen scavenger. In some embodiments, this product may be used as a catalyst-independent oxygen scavenger. In some embodiments, the product upon oxidation changes color and may be used as an oxygen scavenger with built-in shelf-life indicator. For example, when the oxygen scavenger capacity is color changes as the portions of the product is oxidized and lose further scavenging capacity. When the color change cease, there is no longer any scavenging capacity in the product, and the shelf-life for the content will rapidly deteriorate therefrom.

As described above, the described chemical recycling methods or portions thereof may be conducted, rather than in a conventional organic reaction apparatus, in an extruder using reactive extrusion principles.

As described above, with respect to water pollution, this present disclosure provides materials and methods for fabrication. For example, the mixture of the rest of the waste plastics streams not address above or mixed with those described above may be subject to a heat treatment. In some embodiments, those mixture may include colorants. For example, the application describes a catalytic material and its methods of manufacture. The catalytic material that is capable of being produced from solid wastes or the source materials that turn into solid wastes. This catalytic material can be used to accelerate treatments of polluted water or soil utilizing light energy. With respect to water treatment, activated carbon (or activated charcoal) is often used for its versatility. Unfortunately, the cost of production has largely limited its application, especially in less-affluent countries. Meanwhile, titanium oxide (TiO₂) based photocatalytic treatment of water has gained momentum, at least in laboratory settings. Photocatalytic treatment is one type of chemical treatment utilizing a specialized catalyst and solar radiation as energy source. This specialized catalyst TiO₂, or a photocatalyst, is a catalytic material that absorbs light energy and causes the reaction to go faster than it otherwise will go. For example, in a water treatment reaction, organic pollutants may be decomposed under light irradiation at a very slow rate. With a photocatalyst, such reactions may substantially complete within a day, or even within an hour. Beyond wastewater treatment, photocatalysis has been shown promise in treating polluted soil as well. Nevertheless, while photocatalysis is prevalent in laboratory research, industrial applications still suffer from low efficiency and high costs. Scientists and engineers have focused on several paths to address this problem although none has provided a satisfactory solution. The energy absorbed from the light is used by the catalysts to drive the decontamination reactions. However, the most efficient photocatalysts so far, such as titanium dioxide (TiO₂), absorb only light energy in the ultraviolet region which constitutes a small fraction of the entire solar spectrum. Therefore, alternative photocatalysts which covers a wider region of the solar spectrum is likely needed in order to solve this issue. Apart from titanium dioxide (TiO₂), zinc oxide (ZnO), zinc sulfide (ZnS), tungsten trioxide (WO₃), cadium sulfide (CdS), bismuth vanadate (BiVO4), silver phosphate (Ag₃PO₄), manganese titanate (MnTiO₃), Strontium titanate (SrTiO₃), Gallium Nitride (GaN), etc., have all been reported to possess certain photocatalytic properties, although not necessarily in the area of water or soil decontamination. Controlled doping of metal ions into TiO₂ has also been shown to be effective in increasing light absorptions under proper conditions. However, field applications of these materials remain unknown probably due to the overall low treatment outputs derived from the intrinsic low efficiency of these catalysis reactions despite the larger light energy input. High cost of production may also be a factor.

Embodiments of this invention further provide methods of producing catalytic materials, including but not limited to those strongly colored or those with large amounts of additives, from solid wastes. Embodiments of this invention also teach using these materials as photocatalysts to accelerate decontamination reactions of polluted water or soil. With respect to manufacturing methods, like most chemical reactions, the synthesis of TiO₂ typically requires pure starting materials. Impurities often interfere with the reaction progress and cause unexpected defects. This interference is particularly significant when the target application is in catalysis. Small amount of impurities often causes fatal performance problem in catalytic reactions—a problem known as “catalyst poisoning effect”. For example, it is reported that impurities in the TiO₂ structure may cause recombination of radicals (the active species used to treating the pollutants) thereby reducing the number of active radicals hence the efficiency. It is therefore generally considered to be necessary to avoid impure starting materials in preparing photocatalysts. However, certain chemical reactions can start from impure or even waste materials. In fact, waste incineration and pyrolysis are known to be common processes used at MWPFs, often producing valuable gasoline-type final products.

As the brief survey above clearly suggests, environmental remediation is an extremely vast task. Scientists and engineers from different technical background and skill sets are needed to contribute their perspectives and experiences. Traditionally, however, with individualized educational backgrounds, scientists and engineers typically focus their effort in a small technological area. This is also necessitated by the fact that scientists need to focus on smaller subjects such that they have enough time and resources to address specific problems, which alone is effort intensive and require specialized skillsets. Therefore, a person of ordinary skill in the art would typically not cross the borderlines between these different areas.

Embodiments of the present disclosure teaches a composition of photocatalyst, a method to make this composition, a method to use this composition, and a system to implement this method.

While the various descriptions and embodiments focus on using waste products to produce this composition, the invention may be performed with the corresponding virgin materials or recycled material. Accordingly, the use of such virgin or recycled materials shall be covered under the scope of this patent. For example, if an embodiment described below discloses using waste paper as a starting material to prepare a composition, a person of ordinary skills in the art understands that the same may be achieved by using virgin paper, or recycled paper. The recycled paper may has been recycled once, twice or more. For another example, if an embodiment described below discloses using waste polyethylene terephthalate material with a white colorant in it as a starting material to prepare a composition, the same may be achieved by using virgin polyethylene terephthalate material or recycled polyethylene terephthalate material, in conjunction with a white colorant, or a white colorant precursor, added in together or separately. For yet another example, if an embodiment described below discloses using food waste as a starting material, the same may be achieved by using fresh food materials from plant or animal sources.

In addition, if an embodiment described below discloses using one type of waste polymer, the same may be achieved with other waste polymers or other types of waste polymers. Furthermore, if an embodiment described below discloses using waste material from municipal or household origins, commercial or industrial wastes can similarly be used as an alternative when appropriate.

For still another example, if an embodiment described below discloses using a nanomaterial format of TiO₂ in its rutile crystal structure as a starting material, the same may be achieved by use of TiO₂ in other formats or other crystal structure. Similarly, if the description does not disclose the specific format or crystal structure of the TiO₂, any format or crystal structure should be considered to be applicable. If an embodiment refers to char, it may mean coal, coke, or other carbonaceous materials. If the description refers to powder, it should be understood that thin films comprising the powder also possesses the same functionality. If an embodiment refers to a fixed bed pyrolysis reactor, it may also use instead, a batch reactor, a fluidized red reactor, a spouted bed reactor, a rotary kiln reactor, microwave assisted reactor, plasma reactor or solar reactor. If the description does not specify whether a reaction occurs in presence of oxygen or air or other reactive gases, it should be interpreted that any of these situations function for the targeted application. If the embodiment provides one reaction condition, it is understood that the condition may be altered or optimized by routine experimentation according to the principles taught in this disclosure. These general principles apply to other materials described throughout this disclosure.

One embodiment of the instant invention is to use solid wastes as a starting material to prepare catalysts comprising a photocatalyst material and activated carbon. The solid wastes may be waste plastics from bottles, textiles, car parts, cushion materials, containers, etc. It can also be waste paper products such as used multi-purpose paper, used cardboard boxes, used coffee cups, used newspapers, etc. It can also be biomass such as plants, used wood products. It can further be food wastes or food processing wastes enriched with polymeric components such as orange peels (cellulose), wheat straw (lignin), shrimp shells (chitin/chitosan), etc.

In one aspect of the instant invention, we produce a composition of TiO₂ and carbonaceous material. Many examples are provided below for illustrative purposes. They are intended to only be exemplary and shall not be construed to be limiting in any way or form. These representative examples are applicable to other members of the described genus. A person of ordinary skill in the art will be able to make and use embodiments of the invention without undue experimentation.

Example 1. In one embodiment, the following steps are followed: 2 g of waste paper is pulverized and rinsed with water. A Titanium (IV) solution is prepared by dissolving 2 g of Titanium (IV) isopropoxide in a mixture of 400 g deionized water and 20 g acetic acid. After stirring for 3 h, the pulverized waste paper powder is introduced into the solution. The mixture is further stirred and heated at 80° C. until gel formation completes. The gel is dried in the oven at 80° C. for overnight and subsequently is grounded and calcined in a muffle furnace at 500° C. for 5 hours.

Example 2. In another embodiment, the following steps are followed: 2 g of waste paper is pulverized and rinsed with water. An aqueous suspension is prepared by adding 2 g titanium dioxide (TiO₂) nanomaterial into 10 mL of deionized water and well-dispersed by sonication. The pulverized waste paper powder is then introduced into the suspension and re-dispersed with sonication. The suspension is then filtered through a filter paper. The solid residue is transferred into a suitable container and placed in an oven at 80° C. for overnight. The residue is grounded and calcined in a muffle furnace at 500° C. for 5 hours.

Example 3. The same process as in Example 2 is provided except that the TiO₂ is of a porous format. This porous TiO₂ may be prepared before any of the steps recited above by the following steps: 8 g of TiO₂ powder (0.3 mm diameter) is first soaked in 1M NaOH solution and rinsed to clean the powder. Once rinsed, the TiO₂ powder is mixed with 3 mL deionised water and grounded for 1 h. An additional 3 g of TiO₂ powder along with 13 mg of polysaccharide binder is added and further ground for 15 min. A piece of polyurethane foam with open cell structure of approximately 5 mm in diameter is dipped into the slurry and maintained for 1 h. The foam is then centrifuged to remove extra water. The piece is then dried on top of a porous ceramic plate at room temperature for overnight. The piece is then transferred into a programmable oven and undergo the following heating cycle: slowly heating from room temperature to 450° C. at 0.5° C./min, maintain the temperature for 1 h; heating to 1100° C. at 3° C./min, maintain for 10 min; cooling to room temperature at 5° C./min.

Example 4. The same process as in either of Examples 1-3 is provided except that the temperature of oven used for the final calcination step is not set at a constant 500° C. temperature. Instead, the temperature of the oven is programmed according to the following parameters: from 40° C. to 500° C. at 25° C./min, hold at 500° C. for 20 min, cool down to room temperature at 5° C./min. This may be performed in air, in oxygen, or in other gases.

Example 5. The same process as in either of Examples 1-3 is provided except that the calcination temperature used for the final calcination step is not set at a constant 500° C. temperature. Instead, the temperature is programmed according to the following parameters: from 40° C. to 500° C. at 1000° C./s, hold at 500° C. for 3 s, cool down to room temperature at 100° C./min. This can be achieved by, for example, CDS pyrolyzer 5200.

Example 6. The same process as in either of Examples 1-3 is provided except that the temperature used for the final calcination step is not set at a constant 500° C. temperature. Instead, the temperature is programmed according to the following parameters: from 40° C. to 800° C. at 1000° C./s, hold at 800° C. for 0.05 s, cool down to room temperature at 100° C./min. This can be achieved by, for example, CDS pyrolyzer 5200.

Example 7. The same process as in any of Examples 1-6, except that instead of using an oven, the material is heated in a pyrolysis equipment where the gas phase product goes through a condenser, where both the condensed liquid and the uncondensable gas are collected. For example, the equipment used is a commercial scale RES Polyflow system, or a comparable equipment that accommodates the use of catalysts.

Example 8. The same process as in Example 7, except that a pyrolysis catalyst is also included in the composition to undergo the heating process. For example, soluble inorganic catalysts (such as alkaline chlorides, alkaline earth metal chlorides, iron sulfate), zeolite catalysts, FCC catalysts, silica-alumina catalysts, microporous and mesoporous catalysts, or combinations thereof may be used. The appropriate amount of catalyst is heavily dependent upon the type of feedstocks. A person of ordinary skill is capable of conducting routine experimentation to arrive at an optimal dosing of the catalysts. Without limiting, 1-5 wt % KCl, 1-10 wt % of Na2CO3, 1-20 wt % of ZSM-5, 1-50 wt % of FCC, 20-60 wt % of Y-Zeolite, etc. may be used.

Example 9. The same process as in any of Examples 1-8 is used, except that pulverized waste polyolefin plastics is used in place of the pulverized waste paper. For example, waste polystyrene, HDPE, LDPE, LLDPE, polypropylene is used.

Example 10. The same process as in Example 9 is used, except that pulverized waste polyolefin comprises halogens (such as polyvinylchloride) and that a basic material (such as calcium oxide, lead oxide, cerium dioxide, zinc oxide, iron oxides) is added in along with, or separately from, the waste polyolefin, for example, in an amount that is sufficient to neutralize the produced acid. The basic material may serve in conjunction with another catalyst, or serve as the catalyst itself.

Example 11. The same process as in Example 10 is used, except that the waste polyolefin undergoes a two-stage pyrolysis process, first at 300° C. without catalyst, and at the higher temperature(s) with the catalyst.

Example 12. The same process as in any of Examples 1-8 is used, except that pulverized waste condensation polymer, such as polyethylene terephthalate, is used in place of the pulverized waste paper. This may include polyethylene terephthalate, polyamides, polycarbonates.

Example 13. The same process as in any of Examples 1-8 is used, except that pulverized waste biomass, such as wood chips, is used in place of the pulverized waste paper.

Example 14. The same process as in any of Examples 1-13 is used except that the waste starting material is not in a pulverized state, but in chunks, shredded pieces, pellets, pills, etc., and is added in and mixed with the photocatalysts or photocatalyst precursors prior to be calcinated.

Example 15. The same process as in any of Examples 1-14 is used except that the waste starting material is a mixture of two or more of waste paper, waste polyolefin, waste condensation polymers, waste biomass.

In another aspect of the instant invention, we produce a composition of carbonaceous material and other photocatalysts.

Example 16. The same process as in any of Examples 1-15 except that instead of TiO₂ or a TiO₂ precursor, a lead(II) chromate (PbCrO₄) or PbCrO₄ precursor is used.

Example 17. The same process as in any of Examples 1-15 except that instead of TiO₂ or a TiO₂ precursor, a mercury sulfide (HgS) or HgS precursor is used.

Example 18. The same process as in any of Examples 1-15 except that instead of TiO₂ or a TiO₂ precursor, a cadium sulfide (CdS) or CdS precursor is used.

Example 19. The same process as in any of Examples 1-15 except that instead of TiO₂ or a TiO₂ precursor, a different photocatalyst is used, such as iron oxide (FeₓO_{y}), YInMn Blue (YInMn), etc.

Example 20. The same process as in any of Examples 1-15 except that instead of a TiO₂ or TiO₂ precursor, a mixture of two of more of TiO₂, CdS, PbCrO₄, HgS, or another photocatalyst is used.

In yet another aspect of the instant invention, we produce a composition of carbonaceous material and other photocatalysts from colored materials.

Example 21. 2 g of waste plastics containing TiO₂ (such as an opaque white PET bottle pieces) is pulverized and rinsed with water. The solid residue is transferred into a suitable container and placed in an oven at 80° C. for overnight. The residue is grounded and calcinated in a muffle furnace at 500° C. for 5 hours.

Example 22. The same process as in Example 21 is provided except that the temperature of oven is used for the final calcination step not set at a constant 500° C. temperature. Instead, the temperature of the oven is programmed according to the following parameters: from 40° C. to 500° C. at 25° C./min, hold at 500° C. for 20 min, cool down to room temperature at 5° C./min. This may be performed in air, in oxygen, or in other gases.

Example 23. The same process as in Example 21 is provided except that the calcination temperature is used for the final calcination step not set at a constant 500° C. temperature. Instead, the temperature is programmed according to the following parameters: from 40° C. to 500° C. at 1000° C./s, hold at 500° C. for 3 s, cool down to room temperature at 100° C./min. This can be achieved by, for example, CDS pyrolyzer 5200.

Example 24. The same process as in Example 21 is provided except that the temperature is used for the final calcination step not set at a constant 500° C. temperature. Instead, the temperature is programmed according to the following parameters: from 40° C. to 800° C. at 1000° C./s, hold at 800° C. for 0.05 s, cool down to room temperature at 100° C./min. This can be achieved by, for example, CDS pyrolyzer 5200.

Example 25. The same process as in any of Examples 21-24, except that instead of using an oven, the material is heated in a pyrolysis equipment where the gas phase product goes through a condenser and both the condensed liquid and the uncondensable gas are collected. For example, the equipment used is a commercial scale RES Polyflow system, or a comparable equipment that accommodates the use of catalysts.

Example 26. The same process as in any of Examples 1-25, except that the concentration of photocatalyst is modified: If the concentration of TiO₂ is lower than desired, additional TiO₂ is added and mixed with the pulverized plastics; if the concentration of TiO₂ is higher than desired, additional carbonaceous material is added and mixed with the pulverized plastics.

Example 27. The same process as in either of Examples 25 or 26, except that a pyrolysis catalyst is also included in the composition to undergo the heating process.

Example 28. The same process as in any of Examples 21-26, except that instead of a plastics containing TiO₂, a plastics containing zinc oxide (ZnO), iron oxide (FeₓO_{y}) or other additives, a plastics pipe containing cadmium yellow (CdS) paint, a piece of plywood containing a chrome yellow (PbCrO₄) or vermilion (HgS) paint, or other carbonaceous material with other pigments is used.

Example 29. The same process as in any of Examples 21-26, except that instead of a plastics containing TiO₂, one or more carbonaceous materials containing one or more pigments are used. A person of ordinary skill in the art understands that by using one or more pigment, the system may absorb one or more particular wavelengths of light thereby utilizing the energy input from the Sun not only in the ultraviolate region, but also in the visible regions.

In a further aspect of the instant invention, we produce a composition of carbonaceous material and photocatalyst(s) with enhanced efficacies.

Example 30. The same process as in any of Examples 1-29, except that a FeCl₂ is introduced during the preparation to prepare a composition that includes Fe ions;

Example 31. The same process as in any of Examples 1-29, except that a magnetic chemical is introduced during the preparation to prepare a composition that includes magnetic properties;

Example 32. The same process as in any of Examples 1-31, except that after the programmed heating profile is completed, but before the cooling process starts, another heating step is provided as follows: heat from 500° C. to 800° C. at 10° C./min, hold at 800° C. for 1 h; cool down to room temperature at 5° C./min. This may be performed in air, in oxygen, or in other gases.

Example 33. The same process as in any of Examples 1-32 is used except that after the product is collected, the product goes through a steam activation process.

Example 34. The same process as in Example 33 is used, except that the steam activation process is replaced with one of: a carbon dioxide activation method, air activation method, an alkali metal hydroxides activation method, alkali metal carbonates activation method, and transition metal salt activation method.

Example 35. The same process as in Example 33 is used, except that the steam activation process is replaced with another appropriate activation method for activated carbon is used.

Example 36. The same process as in any of the Examples 1-35, except that the composition is further coated onto a floating substrate, such as clay.

Example 37. The same process as in Examples 1-36, except that the composition is coated onto a transparent substrate, such as a transparent polyacrylonitrile (“PAN”) plastics piece with its surface modified to contain —COOH end groups, such that the photocatalyst-activated carbon particles are tethered on the surface of the PAN piece. A person of ordinary skill in the art understands that the piece floats on the surface of the water with the surface with photocatalyst-activated carbon composite material immersed in water, such that the catalyst may receive maximum amount of sunlight energy input.

In still another aspect of the instant invention, the produced catalysts are used in waste water treatment.

Example 38. The composition according to any of the Examples 1-36 is used as a powder in wastewater treatment, wherein ambient solar illumination or Ultraviolet-A illumination at 30 W/m2 is used as the energy input, and the treatment lasts from 10 min to 24 hours.

Example 39. The composition according to any of the Examples 1-36 is used as a film or coating on a substrate in wastewater treatment. A person of ordinary skill in the art understands that there are many different ways to form a thin film with powdered materials, such as dip-coating, liquid phase deposition, etc.

Example 40. The composition according to any of the Examples 1-36 is used to prepare a membrane, such as a polysulfone membrane containing an effective concentration of 1-5% of the active photocatalyst (such as TiO₂ or CdS). The membrane is used for photocatalysis in wastewater treatment. A person of ordinary skill in the art understands that there are many different ways to form a membrane with powdered materials, such as embedding the polysulfone matrix

Example 41. The method of Example 38, wherein the treatment is conducted in one of a Parabolic Trough Reactor, Concentrating Falling Film Reactor, Compound Parabolic Concentrator, Tubular Reactor, Double-Skin Sheet Reactor, Flat Plate Reactor, Falling Film Photoreactor, Thin Film Cascade Photoreactor, Step Photoreactor, Fountain Photocatalytic Reactor, Slurry Bubble Column Reactor, Flat Plate Column Reactor, Pebble Bed Photoreactor, Flat Packed Bed Reactor.

Example 42. The method of Example 38, but the composition used includes magnetic properties (according to Example 27) is used to treat waste water. After treatment is completed, the magnetically active material is retrieved utilizing magnetic field.

In a further aspect, catalyst produced is used in soil remediation. Example 43. Soil remediation. The photocatalyst-activated carbon material is used to treat soil contaminated with organic pollutants such as diphenylarsinic acid, and pesticide Diuron (Nortox, 3-(3,4-dichlorophenyl)-1,1-dimethylurea). The composition as in any of Examples 1-31 is mixed with the contaminated soil (containing approximately 20 mg/kg pollutant) and water at a 0.05:1:10 weight ratio. The sample is then subjected to either solar illumination or ultraviolet illumination at approximately 30 W/m2 for 3 hours. A person of skill in the art understands that the condition to treat soils contaminated with different pollutants require different conditions, and that it is routine to perform optimizations on these parameters in order to find the optimal treatment efficiency.

In still another aspect, the instant invention provides a method of recycling solid wastes without the need to separate between food scrap, plastics, paper, cardboards, woods, but only need to separate those from metals and glasses.

Example 44. Solid wastes are collected from households in streams of (1) recyclable-A (including food scrap, non-thin-bag plastics, paper, biomass such as wood products), (2) recyclable-B (including glass and metal), and non-recyclables (including dirt, plastic bags, hangers, etc.). The recyclable-A is processed according to a method as in any of claims 1-32.

Example 45. Solid wastes are collected from households in a single stream without separating trash or recyclables. Oversize objects are removed from the mix. The remaining material is then dumped into a spinning drum which evenly distributes the recyclables onto a conveyor belt. Plastic bags, coat hangers, and other items that might jam up the line, as well as anything that won't fit through the sorter are first removed. The mix then passes through a series of screens, where materials of different size and shape are separated. Glass is separated from plastic and aluminum due to its weight and falls through the star screens and lands in bins below. Magnetic metal sorter passes above the conveyor and attracts anything magnetic, while aluminum is pushed off the main conveyor. The mix is then substantially free of glass and metal, and is used, according to a method as in any of claims 1-37.

As described above, initial separation of waste plastics may be beneficial for the subsequent chemical processes in some embodiments. While separations amongst several resins are common in the industry, separations amongst others are more challenging. In one embodiment of the present disclosure, different resins are manufactured (or synthesized) with a marker molecule (either small molecule or polymer) which has a unique spectroscopic identifier. The marker molecule may be incorporated onto each monomer unit of the polymer, or may instead be incorporated onto a subset of the monomer unit. The marker molecule may also be incorporated as a comonomer. Alternatively, the marker molecule may be incorporated after the polymer synthesis is completed (i.e. post-synthesis modification or derivatization). In some embodiments, the methods described above for derivatizing polymers may be used to form the marker molecule. In some embodiments, the marker molecule may be fluorescent upon external simulation. Fluorescence is beneficial in that they are unique in wavelengths and not subject to interference of other wavelengths. In some embodiments, phosphorescence is used instead for its longer lifetimes and better stability towards quenching. The marker molecule is configured to be stable, be able to withstand regular processing conditions common to the resins they mark, and be inert to the interferences from common additives to the resin and from common ingredients the resin may be exposed to. Accordingly, each different resin may be marked with a different and specific marker molecule such that they may be rapidly identified during the separation process. Additionally, certain additives may be required to include a unique markers to signify their presence. For example, additives that may be particularly detrimental to a recycling stream may be included such that intermixing may be prevented before they occur using simple prescreen methods.

The above examples are provided merely for illustrative purposes. A person of ordinary skill in the art understands that these examples may be modified in various aspects to optimize the formulation for various different applications without departing from the spirit of the invention. Additionally, these examples shall not be limited to the contexts in which they are described. Rather, they shall be deemed applicable to those additional applications with similar reactive environments to those described here. Moreover, embodiments described here in the context of one application may be combined or modified to address another application. For example, as described above, the method for recycling a plastic material may be related to a method of producing an oxygen scavenger, and/or to producing wastewater treatment photocatalysts. For example, a method to prepare an oxygen scavenger material from a commercial raw material may be modified to instead utilize a waste stream as the raw. Other embodiments of the invention, while not specifically described, will become apparent to those skilled in the art from reading the disclosure and applying the disclosure to experiments.

A first embodiment, which is a compound, comprising a segment having the formula (AA) or (BB):

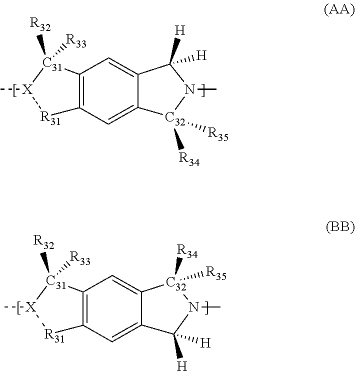

wherein C₃₁ and C₃₂ are carbons (C), wherein R₃₂ and R₃₃ are both hydrogens or collectively forms a carbonyl with C₃₁, wherein R₃₄ and R₃₅ are both hydrogens or collectively forms a carbonyl with C₃₂, wherein X is oxygen (O) or nitrogen attached to an organic residue, wherein R₃₁ is a hydrogen, a hydroxymethyl, a halogen-substituted methyl, or an unsubstituted methylene —CH₂— group covalently and directly bonded to X, wherein dashed lines (---) represent optional covalent bonds to satisfy valence.

A second embodiment, which is the compound of the first embodiment, wherein the segment is covalently bonded to a polymeric backbone, the polymeric backbone having a same structure as the base polymer.

A third embodiment, which is the compound of the first or the second embodiment, wherein each of R₃₂, R₃₃, R₃₄, and R₃₅ are part of a respective carbonyl group.

A fourth embodiment, which is the compound of any of the first through the third embodiments, wherein X is an oxygen of a phthalide fragment.

A fifth embodiment, which is the compound of any of the first through the fourth embodiments, wherein X, R₃₂, R₃₃ and C₃₁ collectively forms an amide (—C(═O)—N—) group of a polyamide backbone.

A sixth embodiment, which is the compound of any of the first through the fifth embodiments, wherein X, R₃₂, R₃₃ and C₃₁ collectively forms a carboxy (—COO—) group of a polyester backbone.

A seventh embodiment, which is the compound of the sixth embodiment, wherein the polyester is one of polyethylene terephthalate, polybutylene terephthalate, polyethylene furanoate, polybutylene adipate terephthalate, or copolymers thereof.

An eighth embodiment, which is the compound of any of the first through the seventh embodiments, wherein X is nitrogen not directly bonded to R₃₁.

A ninth embodiment, which is the compound of any of the first through the eighth embodiments, wherein X is nitrogen and is directly bonded to R₃₁.

A tenth embodiment, which is the compound of any of the first through the ninth embodiments, comprising a phthalic anhydride group.

An eleventh embodiment, which is the compound of any of the first through the tenth embodiments, comprising an oligomer having 1 to 10 repeating units of the formula (AA) or (BB).

A twelfth embodiment, which is the compound of any of the first through the eleventh embodiments, wherein the segment includes a plurality of repeating units, each of a first comonomer moiety condensed with a second comonomer moiety, wherein the first comonomer moiety is different from the second comonomer moiety, and the first comonomer moiety each has the formula (AA) or (BB).

A thirteenth embodiment, which is the compound of the second or the twelfth embodiment, wherein the second comonomer moiety is a diamine or diol.

A fourteenth embodiment, which is the compound of the thirteenth embodiment, wherein amine nitrogens of the diamine or oxygens of the diol are each directly bonded to a substituted or unsubstituted —CH₂—CH₂— fragment.

A fifteenth embodiment, which is the compound of the thirteenth or the fourteenth embodiment, wherein amine nitrogens of the diamine or oxygens of the diol are each directly bonded to a respective allyl or benzyl group.

A sixteenth embodiment, which is the compound of any of the first through the fifteenth embodiments, further comprising a repeating unit of ω-hydroxy-alkylamine (—O—(CH₂)ₙ—NR₃₆—), wherein n is an integer between 1 and about 10, and R₃₆ being any organic residue.

A seventeenth embodiment, which is the compound of the sixteenth embodiment, wherein the repeating unit of ω-hydroxy-alkylamine (—O—(CH₂)ₙ—NR₃₆—) is directly bonded to carbonyl groups on both of its terminals forming amide or ester groups.

An eighteenth embodiment, which is the compound of any of the thirteenth through the fifteenth embodiments, wherein R₃₄, R₃₅ are both hydrogens, and the nitrogen directly bonded to C₃₂ is directly attached to a carbonyl.

A nineteenth embodiment, which is the compound of any of the first through the eighteenth embodiments, wherein the X and the N of formula (AA) or (BB) are each directly bonded to a carbon terminal of —(CH₂)ₙY—R₃₆, wherein n is an integer between 1 and about 10, Y is oxygen or —NR₃₆—, and each occurrence of R₃₆ is independently chosen from a hydrogen and an organic residue.

A twentieth embodiment, which is the compound of any of the first through the nineteenth embodiments, wherein the segment includes a fused three-ring structure.

A twenty-first embodiment, which is the compound of any of the first through the twentieth embodiments, comprising a ditopic diphthalimidino-moiety (-DP-) having a benzene ring simultaneously fused with two 5-member rings to form two phthalimidine structures sharing the benzene ring.

A twenty-second embodiment, which is the compound of any of the first through the twenty-first embodiments, wherein R₃₂, R₃₃, R₃₄, and R₃₅ are hydrogens.

A twenty-third embodiment, which is the compound of any of the first through the twenty-second embodiments, wherein the segment is an end group to a polyester.

A twenty-fourth embodiment, which is the compound of any of the first through the twenty-third embodiments, wherein the segment is bonded to a polyethylene terephthalate backbone.

A twenty-fifth embodiment, which is the compound of any of the first through the twenty-fourth embodiments, wherein X is an oxygen and R₃₁ is hydrogen.

A twenty-sixth embodiment, which is the compound of any of the first through the twenty-fifth embodiments, wherein the segment is a first segment of the compound, and the compound includes a second segment having the formula (AA) or (BB), wherein X of the first segment and X of the second segment are different.

A twenty-seventh embodiment, which is the compound of any of the first through the twenty-sixth embodiments, comprising a plurality of ditopic diphthalimidino-moieties (-DP-) and a phthalide moiety.

A twenty-eighth embodiment, which is the compound of any of the first through the twenty-seventh embodiments, comprising a plurality of ditopic diphthalimidino-moieties (-DP-) each directly bonded to alkylene —(CH₂)ₙ— groups forming -[DP-(CH₂)ₙ]ₘ— oligomers or polymers, wherein n is an integer of 1 to about 10, and m is any integer.

A twenty-ninth embodiment, which is the compound of any of the first through the twenty-eighth embodiments, comprising a plurality of ditopic diphthalimidino-moieties (-DP-) each directly bonded to xylyl —CH₂ArCH₂— groups forming -[DP-CH₂ArCH₂—]ₘ— oligomers or polymers, wherein Ar is a benzene or —CH═CH—, and m is any integer.

A thirtieth embodiment, which is the compound of any of the first through the twenty-ninth embodiments, comprising a plurality of ditopic diphthalimidino-moieties (-DP-) each directly bonded to an alkyl terminal of a —(CH₂)ₙO—C(═O)— group on both terminals of the -DP-, wherein n is 1 to about 10.

A thirty-first embodiment, which is the compound of any of the first through the thirtieth embodiments, comprising trimellitic anhydride group.

A thirty-second embodiment, which is the compound of the thirty-first embodiment, comprising a polyester segment directly attached to a carboxy oxygen of the trimellitic anhydride group.

A thirty-third embodiment, which is the compound of any of the first through the thirty-second embodiments, comprising a fragment of —CC-(DD-CC)ₙ—, wherein n is any integer, and at least one of CC and DD has the formula of (AA) or (BB).

A thirty-fourth embodiment, which is the compound of any of the first through the thirty-third embodiments, comprising a fragment of -EE-(CC-DD-CC-EE)ₘ-, wherein at least one of CC and DD has the formula of (AA) or (BB), m is any integer, and EE is a repeating unit of a condensation polymer.

A thirty-fifth embodiment, which is the compound of any of the thirty-third and the thirty-fourth embodiments, wherein at least one of CC and DD includes an allyl or benzyl.

A thirty-sixth embodiment, which is the compound of any of the first through the thirty-fifth embodiments, wherein the X is oxygen and is directly attached to —(CH₂)₂—.

A thirty-seventh embodiment, which is the compound of any of the first through the thirty-sixth embodiments, wherein the X is nitrogen and is directly attached to a benzyl or allyl group.

A thirty-eighth embodiment, which is a compound, comprising a segment having the formula (LL), (MM), or (NN):

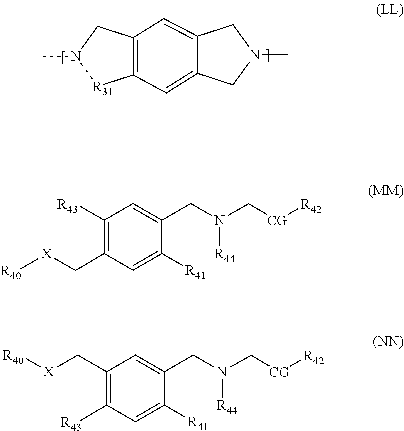

wherein R₃₁ is a hydrogen, a hydroxymethyl, a halogen-substituted methyl, or an unsubstituted methylene —CH₂— group covalently and directly bonded to X, wherein CG represents benzene or —C═C— double bond, X is oxygen or NR₄₅, R₄₄ and R₄₅ are each independently a hydrogen or any organic residue, R₄₁ and R₄₃ are each independently selected from hydrogen, carboxy group, ester group, and amide group, wherein R₄₅ is optionally connected with R₄₃ to form a ring with X, and wherein R₄₁ is optionally connected with R₄₄ to form a ring with the N atom of the formula (FF) or (GG), and wherein dashed lines (---) represent optional covalent bonds to satisfy valence.

A thirty-ninth embodiment, which is a compound, comprising having a formula of (FF) or (GG).

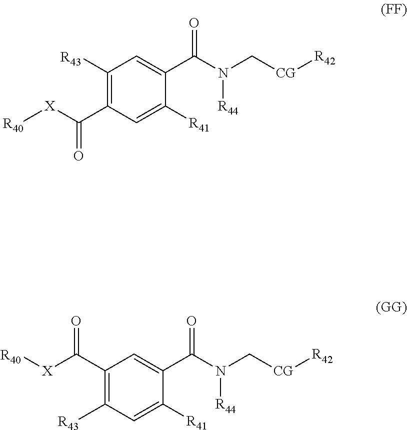

wherein CG represents benzene or —C═C— double bond, X is oxygen or NR₄₅, R₄₄ and R₄₅ are each independently a hydrogen or any organic residue, R₄₁ and R₄₃ are each independently selected from hydrogen, carboxy group, ester group, and amide group, wherein R₄₅ is optionally connected with R₄₃ to form a ring with X, and wherein R₄₁ is optionally connected with R₄₄ to form a ring with the N atom of the formula (FF) or (GG).

A fortieth embodiment, which is the compound of any of the thirty-eighth and the thirty-ninth embodiments, wherein R₄₂ includes formula (HH) or (JJ):

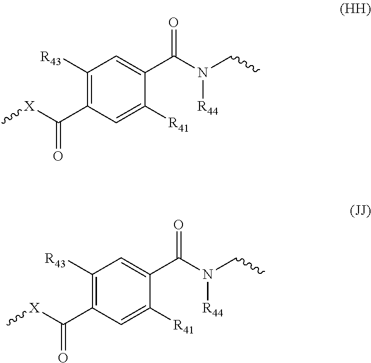

A forty-first embodiment, which is the compound of any of the thirty-eighth through the fortieth embodiments, wherein the R₄₀ includes a polyester oligomer or polymer.

A forty-second embodiment, which is the compound of any of the thirty-eighth through the forty-first embodiments, wherein X is oxygen.

A forty-third embodiment, which is the compound of any of the thirty-eighth through the forty-second embodiments, wherein R₄₄ includes —CH₂—CG-.

A forty-fourth embodiment, which is the compound of any of the thirty-eighth through the forty-third embodiments, wherein R₄₄ directly bonds to CG. A forty-fifth embodiment, which is the compound of any of the thirty-eighth through the forty-fourth embodiments, wherein R₄₂ includes —CH₂—Y—, wherein Y is oxygen or NR₄₅, R₄₅ is hydrogen or any organic residue.

A forty-sixth embodiment, which is the compound of any of the thirty-eighth through the forty-fifth embodiments, wherein R₄₂ includes a polyester oligomer or polymer.

A forty-seventh embodiment, which is the compound of any of the thirty-eighth through the forty-sixth embodiments, wherein X is NR₄₅.

A forty-eighth embodiment, which is the compound of any of the thirty-eighth through the forty-seventh embodiments, wherein R₄₀ includes —CH₂—CG-.

A forty-ninth embodiment, which is the compound of any of the thirty-eighth through the forty-eighth embodiments, wherein at least one of R₄₁ and R₄₃ is not hydrogen.

A fiftieth embodiment, which is the compound of any of the thirty-eighth through the forty-ninth embodiments, wherein at least one of R₄₁ and R₄₃ includes a polyester oligomer or polymer.

A fifty-first embodiment, which is the compound of any of the thirty-eighth through the fiftieth embodiments, wherein R₄₁ and R₄₃ each include a polyester oligomer or polymer.

A fifty-second embodiment, which is the compound of any of the first through the fifty-first embodiments, further comprising an activator moiety selected from the following:

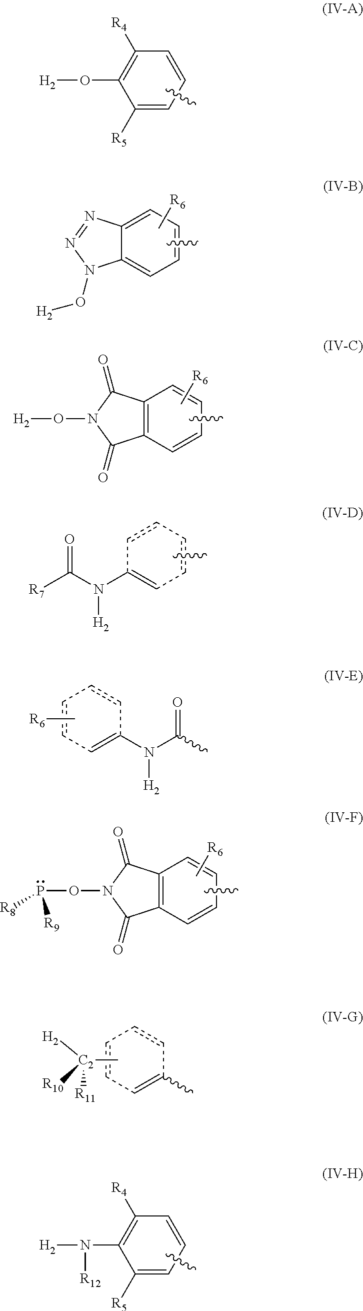

wherein R₄-R₁₂ may each be selected independently from a hydrogen, an organic residue, and an organometallic residue, and where H2 is hydrogen.

A fifty-third embodiment, which is the compound of the fifty-second embodiment, wherein at least one of R₄ and R₅ has a Van de Waals radius greater than that of an isopropyl group.

A fifty-fourth embodiment, which is the compound of any of the fifty-second and the fifty-third embodiments, wherein R₄ or R₅ includes a methylthio group —SCH₃.

A fifty-fifth embodiment, which is the compound of any of the fifty-second through the fifty-fourth embodiments, wherein the activator moiety is conjugatively attached to a —CH₂— unit.

A fifty-sixth embodiment, which is the compound of any of the fifty-second through the fifty-fifth embodiments, wherein the activator moiety includes a segment of formula (III):

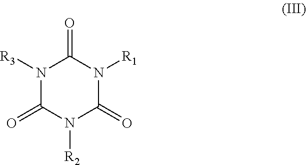

wherein R₁, R₂, and R₃ may each be an allyl group.

A fifty-seventh embodiment, which is a method of preparing the compound of any of the first through the fifty-sixth embodiments, wherein the preparing includes reacting with one of terephthalic acid, isophthalic acid, phthalic acid, terephthalic ester, isophthalic ester, and phthalic ester.

A fifty-eighth embodiment, which is the method of the fifty-seventh embodiment, wherein the preparing includes reacting with terephthalic acid.

A fifty-ninth embodiment, which is a method of preparing terephthalic acid of the fifty-eighth embodiment, wherein the preparing of the terephthalic acid includes preparing from a polymer having a 1,4-benzenedicarbonyl segment, comprising subjecting the polymer to a mechanochemical condition with an inorganic bicarbonate in presence of a water molecule.

A sixtieth embodiment, which is a method of preparing terephthalic acid of the fifty-eighth embodiment from a polymer having a 1,4-benzenedicarbonyl segment, comprising reactively extruding the polymer with an inorganic hydroxide.

A sixty-first embodiment, which is a method of preparing terephthalic acid of the fifty-eighth embodiment from a polymer having a 1,4-benzenedicarbonyl segment, comprising subjecting the polymer to a mechanochemical condition with an inorganic formate in presence of a water molecule.

A sixty-second embodiment, which is the method of any of the fifty-ninth through the sixty-first embodiments, wherein the polymer is a part of a mechanically recycled plastics.

A sixty-third embodiment, which is the method of the sixty-second embodiment, wherein the mechanically recycled plastics is a residue material from multiple mechanical recycling and no longer suitable for mechanical recycling.

A sixty-fourth embodiment, which is a composition, comprising a base polymer and a molecule of formula (I) and (II):

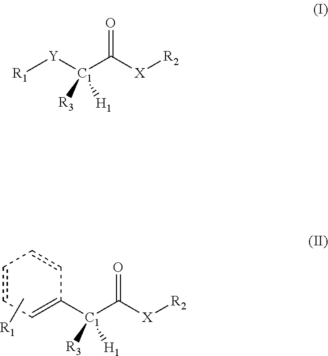

where X and Y are each independently chosen from oxygen (O) and NR₄₆, and R₂, R₃, R₄₆ are each independently hydrogen or any organic residue, C₁ is carbon, and H₁ is hydrogen.

A sixty-fifth embodiment, which is the composition of the sixty-fourth embodiment, wherein the molecule is one of glycine, glycinamide, and glycolamide.

A sixty-sixth embodiment, which is the composition of the sixty-fourth or the sixty-fifth embodiment, wherein R₃ is one of benzene and a —CH═CH—.

A sixty-seventh embodiment, which is the composition of any of the sixty-fourth through the sixty-sixth embodiments, wherein the molecule is a cyclic anhydride.

A sixty-eighth embodiment, which is the composition of any of the sixty-fourth through the sixty-seventh embodiments, wherein the molecule is an oligomer or polymer of formula (I) or (II) as a monomer or comonomer.

A sixty-ninth embodiment, which is the composition of any of the first through the sixty-eighth embodiments, further comprising a radical-based oxidation catalyst.

A seventieth embodiment, which is the composition of any of the first through the sixty-eighth embodiments, comprising no radical-based oxidation catalyst.

A seventy-first embodiment, which is the composition of any of the first through the seventieth embodiments, for a shelf-life extension application.

A seventy-second embodiment, which is the composition of any of the first through the seventy-first embodiments, for stabilizing a polymer against heat or light.

A seventy-third embodiment, which is a composition, consisting essentially of (1) a base polymer, (2) the compound of any of the first through the fifty-sixth embodiments, or the molecule of any of the sixty-fourth through the sixty-eighth embodiments, and (3) optionally a metal-based exchange catalyst.

A seventy-fourth embodiment, which is the compound of any of the first through the fifty-sixth embodiments, wherein the segment is a sidechain or attached to a sidechain of a polymer.

A seventy-fifth embodiment, which is the compound of any of the first through the fifty-sixth embodiments, comprising an oligomer of one of polyethylene terephthalate, polybutylene terephthalate, polyethylene furanoate, polybutylene adipate terephthalate, or copolymers thereof.

A seventy-sixth embodiment, which is the compound of any of the first through the fifty-sixth embodiments, further comprising a fragment having a monomer unit of a base polymer surrounding the compound in an article.

### INDUSTRIAL APPLICABILITY

Embodiments of this present disclosure may be used in various packaging or other polymer applications to extend the useful life of the polymers themselves by structurally or compositionally modifying them at the material manufacturing stage, article manufacturing stage, application stage, mechanical recycling stage, and chemical recycling stage. Moreover, embodiments of this disclosure further may be used to improving the capability of the packaging materials in protecting contents they contain. The end result is that various resources are conserved.

1. A compound, comprising:
(1) a segment having the formula (AA), (BB), (FF), (GG), (MM), (NN), each with an X-end and an N-end: 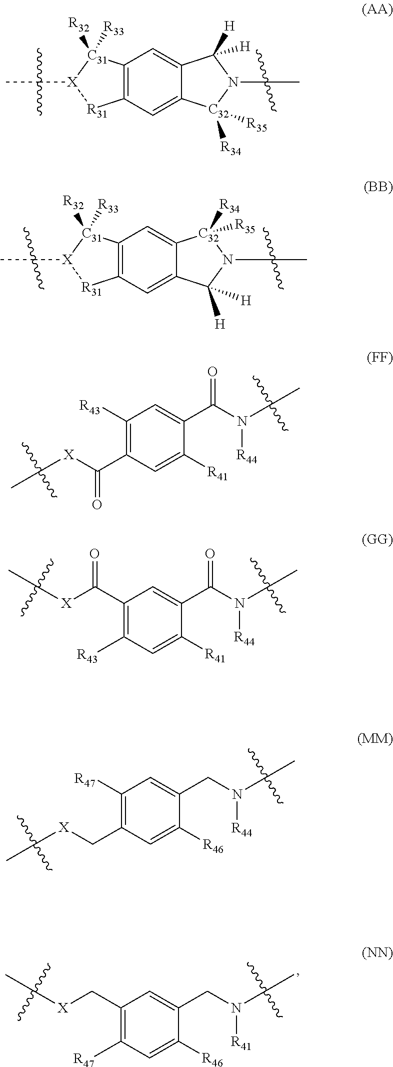
(2) a segment having the formula (KK) with an N-end: 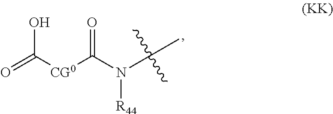
or
(3) a segment having the formula (PP) or (QQ) each with an X-end: 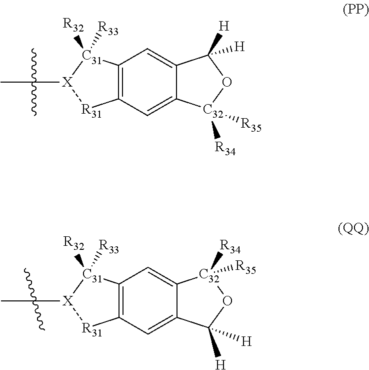
wherein the N-end of formula (FF), (GG), (MM), and (NN) are each directly and covalently bonded to —CH₂—CG-,
wherein C₃₁ and C₃₂ are carbons (C),
wherein R₃₂ and R₃₃ are both hydrogens or collectively forms a carbonyl with C₃₁,
wherein R₃₄ and R₃₅ are both hydrogens or collectively forms a carbonyl with C₃₂,
wherein X is oxygen (O) or Xis nitrogen attached to an organic residue,
wherein R₃₁ is a hydrogen, a hydroxymethyl, a halogen-substituted methyl, or an unsubstituted methylene —CH₂— group covalently and directly bonded to X,
wherein dashed lines (---) represent optional covalent bonds to satisfy valence,
wherein the wavy lines each represent a point of attachment,
wherein CG and CG⁰ are each independently benzene or carbon-carbon double bond,
wherein R₄₁ and R₄₃ are each independently hydrogen, carboxy (—COOH) group, ester (—COOR^{x}) group, or amide (—CONR^{x}R^{y})group, and
wherein R₄₄ is hydrogen or any organic residue, and
wherein R₄₆ and R₄₇ are each independently hydrogen, hydroxymethyl group, alkoxymethyl group, aminomethyl group, or alkylaminomethyl,
provided that:
(i) for formula (AA) or (BB), when X is not directly and covalently bonded to the R₃₁ and further either
(a) R₃₄ and R₃₅ are both hydrogen, or
(b) (R₃₂, R₃₃, C₃₁) and (R₃₄, R₃₅, C₃₂) each collectively forms a respective carbonyl, then:
(1) the segment is directly and covalently bonded to an oligomeric or polymeric moiety,
(2) the segment is directly and covalently bonded to two diradicals of a same formula on the N-end and on the X-end of the formula (AA) or (BB),
(3) the X-end is directly and covalently bonded to an -alkylene-Y, where Y is a heteroatom,
(4) the N-end of the formula (AA) or (BB) is not directly attached to a benzyl group, or
(5) R₃₁ is not hydrogen,
(ii) for (FF) and (GG), when CG is benzene, then:
(1) the segment is directly and covalently bonded to an oligomeric or polymeric moiety,
(2) at least one of R₄₁ and R₄₃ is carboxy or ester,
(3) X is oxygen,
(4) the X-end is not directly and covalently bonded to a methylene of a benzyl,
(5) the N-end is directly and covalently bonded to the methylene end of formula (HH) or formula (JJ): 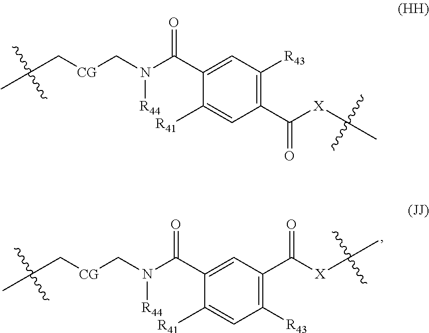
or
(6) R₄₄ is not hydrogen,
(iii) for (MM) and (NN), when R₄₄ is —C(Z¹)(Z²)—Ar, where Z¹ and Z² are independently hydrogen, halogen, C1-C4 alkyl, electronic withdrawing group, electronic donating group, or collectively ═O, and Ar is aryl or heteroaryl, then:
(1) the segment is directly and covalently bonded to an oligomeric or polymeric moiety,
(2) at least one of R₄₆ and R₄₇ is not hydrogen,
(3) X is oxygen, or
(4) the X-end is not simultaneously directly and covalently bonded to two methylene groups, each of the two methylene groups directly and covalently attached to an aryl, and
(iv) for formula (KK), when both CG⁰ and CG are benzene:
(1) the segment is directly and covalently bonded to an oligomeric or polymeric moiety,
(2) CG⁰ is substituted in ortho-pattern, or
(3) the N-end is not directly and covalently bonded to a methylene of a benzyl and not directly and covalently bonded to a methylene of —CH₂-ph-CH₂—NR₄₄—C(═O)-ph-, where ph is phenyl,
wherein R^{x} and R^{y} are each hydrogen or any organic residue.

2. (canceled)

3. The compound of claim 1, the segment having the formula (AA), (BB), (PP), or (QQ), wherein each of R₃₂, R₃₃, R₃₄, and R₃₅ are part of a respective carbonyl group.

4. The compound of claim 1, the segment having the formula (AA) or (BB), wherein X is an oxygen of a phthalide fragment.

5. The compound of claim 1, the segment having the formula (AA), (BB), (PP), or (QQ), wherein X, R₃₂, R₃₃ and C₃₁ collectively forms an amide (—C(═O)—N—) group of a polyamide backbone, or
the segment having the formula (FF), (GG), (MM), or (NN), wherein the X-end forms a portion of an amide bond of a polyamide backbone.

6. The compound of claim 1, the segment having the formula (AA), (BB), (PP), or (QQ), wherein X, R₃₂, R₃₃ and C₃₁ collectively forms a carboxy (—COO—) group of a polyester backbone, or
the segment having the formula (FF), (GG), (MM), or (NN), wherein the X-end forms a portion of an ester bond of a polyester backbone.

7. (canceled)

8. The compound of claim 1, wherein X is nitrogen not directly bonded to R₃₁.

9. The compound of claim 1, wherein X is nitrogen and is directly bonded to R₃₁.

10. The compound of claim 1, comprising a phthalic diacid, phthalimido, or phthalic anhydride group.

11. The compound of claim 1, comprising an oligomer having 2 to 10 repeating units of the formula (AA) or (BB).

12-15. (canceled)

16. The compound of claim 1, the segment having the formula (FF), (GG), (MM), or (NN), wherein R₄₄ includes —CH₂—CG-.

17. The compound of claim 1, the segment having the formula (FF), (GG), wherein at least one of R₄₁, R₄₃, and a moiety directly and covalently bonded to the X-end includes a polyester oligomer or polymer, or
the segment having the formula (MM), or (NN), wherein at least one of R₄₆, R₄₇ and a moiety directly and covalently bonded to the X-end includes a polyester oligomer or polymer.

18-20. (canceled)

21. The compound of claim 1, wherein the segment includes a plurality of repeating units, each of a first comonomer moiety condensed with a second comonomer moiety, wherein the first comonomer moiety is different from the second comonomer moiety, and the first comonomer moiety each has the formula (AA) or (BB).

22. The compound of claim 21, wherein the second comonomer moiety is a diamine or diol.

23. The compound of claim 1, further comprising a repeating unit of ω-hydroxy-alkylamine (—O—(CH₂)ₙ—NR₃₆—), wherein n is an integer between 1 and about 10, and R₃₆ being hydrogen or any organic residue.

24. The compound of claim 23, wherein the repeating unit of ω-hydroxy-alkylamine (—O—(CH₂)ₙ—NR₃₆—) is directly and covalently bonded to carbonyl groups on both of its terminals forming amide or ester groups.

25. The compound of claim 1, comprising a fragment of —CC-(DD-CC)ₙ— or -EE-(CC-DD-CC-EE)ₙ-, wherein n is any integer, and at least one of CC and DD has the formula of (AA) or (BB), and EE is a repeating unit of a condensation polymer.

26. A composition, comprising:
a base polymer; and
(I) a molecule of formula (RR) or (SS): 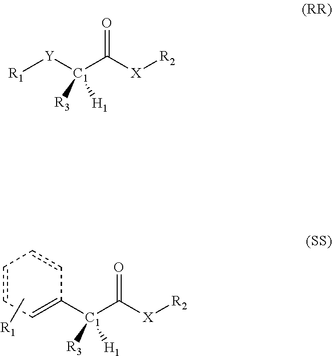
where X and Y are each independently chosen from oxygen (O) and NR₅₀, and R₂, R₃, R₅₀ are each independently hydrogen or any organic residue, C₁ is carbon, and H₁ is hydrogen, or
(II) a compound including:
(1) a segment having the formula (AA), (BB), (FF), (GG), (MM), (NN), each with an X-end and an N-end: 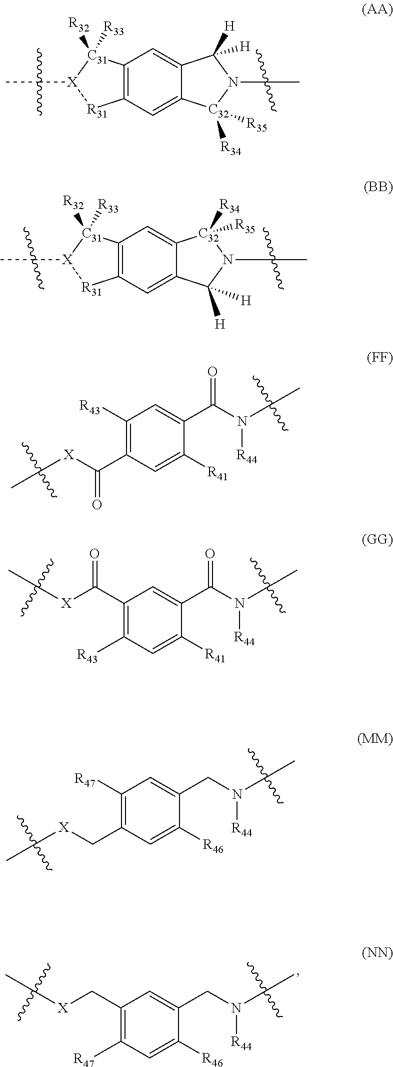
(2) a segment having the formula (KK) with an N-end: 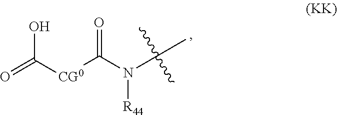
or
(3) a segment having the formula (PP) or (QQ) each with an X-end: 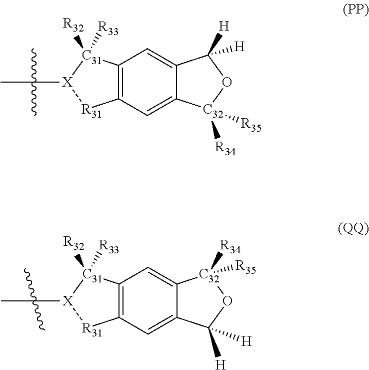
wherein the N end of formula (FF), (GG), (MM), and (NN) are each directly and covalently bonded to —CH₂—CG-,
wherein C₃₁ and C₃₂ are carbons (C),
wherein R₃₂ and R₃₃ are both hydrogens or collectively forms a carbonyl with C₃₁,
wherein R₃₄ and R₃₅ are both hydrogens or collectively forms a carbonyl with C₃₂,
wherein X is oxygen (O) or X is nitrogen attached to an organic residue,
wherein R₃₁ is a hydrogen, a hydroxymethyl, a halogen-substituted methyl, or an unsubstituted methylene —CH₂— group covalently and directly bonded to X,
wherein dashed lines (---) represent optional covalent bonds to satisfy valence,
wherein the wavy lines each represent a point of attachment,
wherein CG and CG⁰ are each independently benzene or carbon-carbon double bond,
wherein R₄₁ and R₄₃ are each independently hydrogen, carboxy (—COOH) group, ester (—COOR^{x}) group, or amide (—CONR^{x}R^{y})group, and
wherein R₄₄ is hydrogen or any organic residue,
wherein R₄₆ and R₄₇ are each independently hydrogen, hydroxymethyl group, alkoxymethyl group, aminomethyl group, or alkylaminomethyl,
provided that:
(i) for formula (AA) or (BB), when X is not directly and covalently bonded to the R₃₁ and further either
(a) R₃₄ and R₃₅ are both hydrogen, or
(b) (R₃₂, R₃₃, C₃₁) and (R₃₄, R₃₅, C₃₂) each collectively forms a respective carbonyl, then:
(1) the segment is directly and covalently bonded to an oligomeric or polymeric moiety,
(2) the segment is directly and covalently bonded to two diradicals of a same formula on the N end and on the X end of the formula (AA) or (BB),
(3) the X-end is directly and covalently bonded to an -alkylene-Y, where Y is a heteroatom,
(4) the N-end of the formula (AA) or (BB) is not directly attached to a benzyl group, or
(5) R₃₁ is not hydrogen,
(ii) for (FF) and (GG), when CG is benzene, then:
(1) the segment is directly and covalently bonded to an oligomeric or polymeric moiety,
(2) at least one of R₄₁ and R₄₃ is carboxy or ester,
(3) X is oxygen,
(4) the X-end is not directly and covalently bonded to a methylene of a benzyl,
(5) the N-end is directly and covalently bonded to the methylene end of formula (HH) or formula (JJ): 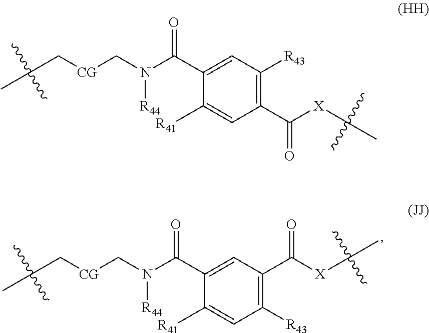
or
(6) R₄₄ is not hydrogen,
(iii) for (MM) and (NN), when R₄₄ is —C(Z¹)(Z²)—Ar, where Z¹ and Z² are independently hydrogen, halogen, C1-C4 alkyl, electronic withdrawing group, electronic donating group, or collectively ═O, and Ar is aryl or heteroaryl, then:
(1) the segment is directly and covalently bonded to an oligomeric or polymeric moiety,
(2) at least one of R₄₆ and R₄₇ is not hydrogen,
(3) X is oxygen, or
(4) the X-end is not simultaneously directly and covalently bonded to two methylene groups, each of the two methylene groups directly and covalently attached to an aryl, and
(iv) for formula (KK), when both CG⁰ and CG are benzene:
(1) the segment is directly and covalently bonded to an oligomeric or polymeric moiety,
(2) CG⁰ is substituted in ortho-pattern, or
(3) the N-end is not directly and covalently bonded to a methylene of a benzyl and not directly and covalently bonded to a methylene of —CH₂-ph-CH₂—NR₄₄—C(═O)-ph-, where ph is phenyl,
wherein R^{x} and R^{y} are each hydrogen or any organic residue.

27. The composition of claim 26, further comprising a radical-based oxidation catalyst.

28. The composition of claim 26, being devoid of a radical-based oxidation catalyst.

29. A compound comprising an activator moiety conjugatively coupled to a first carbon atom having a hydrogen attached thereon, and the activator moiety selected from the following: 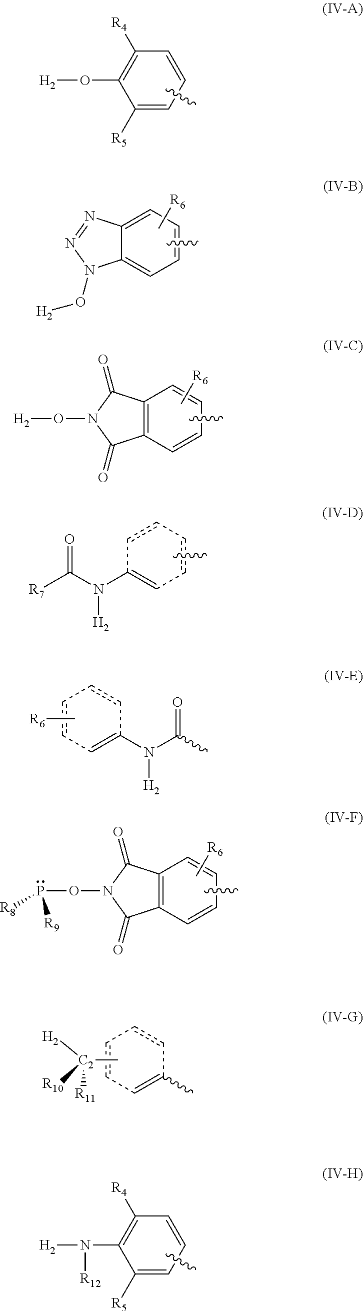
wherein R₄-R₁₂ may each be selected independently from a hydrogen, an organic residue, and an organometallic residue, wherein the symbol H₂ denotes hydrogen atom, and
wherein the first carbon atom is directly and covalently bonded to:
(i) each of a strong mesomeric electron-donating group and a strong mesomeric electron withdrawing group, or
(ii) at feast two atoms each being independently selected from the group consisting of (1) an atom of sp² hybridization, (2) an atom of sp hybridization, and (3) an atom with a lone pair of valence electrons.
